(19) United States
(12) Patent Application Publication
(10) Pub. No.: US 2010/0119599 A1
(43) Pub. Date: May 13, 2010

(54) POLYHYDROQUINOLINE COMPOUNDS AND DIHYDROPYRIDINE COMPOUNDS FOR INHIBITING BETA-AMYLOID PRODUCTION

(75) Inventors: Michael J. Mullan, Tampa, FL (US); Daniel Paris, Sarasota, FL (US); Pancham Bakshi, Sarasota, FL (US)

Correspondence Address: SENNIGER POWERS LLP, 100 NORTH BROADWAY, 17TH FLOOR, ST LOUIS, MO, 63102, US

(73) Assignee: ARCHER PHARMACEUTICALS, INC., Sarasota, FL (US)

(21) Appl. No.: 12/518,251

(22) PCT Filed: Dec. 10, 2007
(86) PCT No.: PCT/US07/87019

§ 371 Date: Dec. 1, 2009

Related U.S. Application Data
(60) Provisional application No. 60/869,243, filed on Dec. 8, 2006.

(51) Int. Cl. A61K 9/64; A61P 25/28; A61P 25/00; A61K 9/12; A61K 31/444; A61K 31/4436; A61K 31/4365

(52) U.S. Cl. 424/456; 424/45; 514/334; 514/337; 514/301

(57) ABSTRACT

Provided are methods of treating or reducing the risk of developing beta-amyloid production, beta-amyloid deposition, beta-amyloid neurotoxicity (including abnormal hyperphosphorylation of tau) and microgliosis associated with cerebral accumulation of Alzheimer's amyloid by administering therapeutically effective amounts of polyhydroquinoline and dihydropyridine compounds which decrease Abeta production in cells. Further provided are methods for diagnosing diseases associated with cerebral accumulation of Alzheimer's amyloid in animals or humans by administering diagnostically effective amounts of polyhydroquinoline and dihydropyridine compounds which decrease Abeta production in cells.

### FIELD OF THE INVENTION

The present invention relates to methods of treatment and diagnosis of diseases associated with cerebral accumulation of Alzheimer's amyloid, such as Alzheimer's disease, using polyhydroquinoline and dihydropyridine compounds provided herein.

### BACKGROUND

Alzheimer's disease (AD) is the most common neurodegenerative disorder of aging, afflicting approximately 1% of the population over the age of 65. Characteristic features of the disease include neurofibrillary tangles composed of abnormal tau protein, paired helical filaments, neuronal loss, and alteration in multiple neurotransmitter systems. The hyperphosphorylation of microtubule-associated tau protein is a known marker of the pathogenic neuronal pre-tangle stage in AD brain (Tan et al., “Microglial Activation Resulting from CD40R/CD40L Interaction after Beta-Amyloid Stimulation,” Science 286:2352-55, 1999).

A significant pathological feature of AD is an overabundance of diffuse and compact senile plaques in association with limbic areas of the brain. Although these plaques contain multiple proteins, their cores are composed primarily of β-amyloid protein, a 39-43 amino acid proteolytic fragment that is proteolytically derived from amyloid precursor protein (APP), a transmembrane glycoprotein. Additionally, C-terminal fragments (CTF) of APP are known to accumulate intraneuronally in AD.

β-amyloid is derived from APP, a single-transmembrane protein with a 590 to 680 amino acid extracellular amino terminal domain and an approximately 55 amino acid cytoplasmic tail. Messenger RNA from the APP gene on chromosome 21 undergoes alternative splicing to yield eight possible isoforms, three of which (the 695, 751 and 770 amino acid isoforms) predominate in the brain. APP undergoes proteolytic processing via three enzymatic activities, termed α-, β- and γ-secretase. Alpha-secretase cleaves APP at amino acid 17 of the β-amyloid domain, thus releasing the large soluble amino-terminal fragment α-APP for secretion. Because α-secretase cleaves within the β-amyloid domain, this cleavage precludes β-amyloid formation. Alternatively, APP can be cleaved by β-secretase to define the amino terminus of β-amyloid and to generate the soluble amino-terminal fragment β-APP. Subsequent cleavage of the intracellular carboxy-terminal domain of APP by γ-secretase results in the generation of multiple peptides, the two most common being a 40 amino acid β-amyloid (Aβ1-40) and 42 amino acid β-amyloid (Aβ1-42). Aβ1-40 comprises 90-95% of the secreted β-amyloid and is the predominant species recovered from cerebrospinal fluid (Seubert et al., Nature, 359:325-7, 1992). In contrast, less than 10% of secreted β-amyloid is Aβ1-42. Despite the relative paucity of Aβ1-42 production, Aβ1-42 is the predominant species found in plaques and is deposited initially, perhaps due to its ability to form insoluble amyloid aggregates more rapidly than Aβ1-40 (Jarrett et al., Biochemistry, 32:4693-7, 1993). The abnormal accumulation of β-amyloid in the brain is believed to be due to decreased clearance of β-amyloid from the brain to the periphery or excessive production of β-amyloid. Various studies suggest excessive production of β-amyloid is due to either overexpression of APP or altered processing of APP, or mutation in the γ-secretases or APP responsible for β-amyloid formation.

β-Amyloid peptides are thus believed to play a critical role in the pathobiology of AD, as all the mutations associated with the familial form of AD result in altered processing of these peptides from APP. Indeed, deposits of insoluble, or aggregated, fibrils of β-amyloid in the brain are a prominent neuropathological feature of all forms of AD, regardless of the genetic predisposition of the subject. It also has been suggested that AD pathogenesis is due to the neurotoxic properties of β-amyloid. The cytotoxicity of β-amyloid was first established in primary cell cultures from rodent brains and also in human cell cultures. The work of Mattson et al. (J. Neurosci., 12:376-389, 1992) indicates that β-amyloid, in the presence of the excitatory neurotransmitter glutamate, causes an immediate pathological increase in intracellular calcium, which is believed to be very toxic to the cell through its greatly increased second messenger activities.

Concomitant with β-amyloid production and β-amyloid deposition, there exists robust activation of inflammatory pathways in AD brain, including production of pro-inflammatory cytokines and acute-phase reactants in and around β-amyloid deposits (McGeer et al., J. Leukocyte Biol. 65:409-15, 1999). Activation of the brain's resident innate immune cells, the microglia, is thought to be intimately involved in this inflammatory cascade. It has been demonstrated that reactive microglia produce pro-inflammatory cytokines, such as inflammatory proteins and acute phase reactants, such as alpha-1-antichymotrypsin, transforming growth factor β, apolipoprotein E and complement factors, all of which have been shown to be localized to β-amyloid plaques and to promote β-amyloid plaque “condensation” or maturation (Nilsson et al., J. Neurosci. 21:1444-5, 2001), and which at high levels promote neurodegeneration. Epidemiological studies have shown that patients using non-steroidal anti-inflammatory drugs (NSAIDS) have as much as a 50% reduced risk for AD (Rogers et al., Neurobiol. Aging 17:681-6, 1996), and post-mortem evaluation of AD patients who have undergone NSAID treatment has demonstrated that risk reduction is associated with diminished numbers of activated microglia (Mackenzie et al., Neurology 50:986-90, 1998). Further, when Tg APPsw mice, a mouse model for Alzheimer's disease, are given an NSAID (ibuprofen), these animals show reduction in β-amyloid deposits, astrocytosis, and dystrophic neurites correlating with decreased microglial activation (Lim et al., J. Neurosci. 20:5709-14, 2000).

At present, treatment for AD is limited. However, there are several drugs approved by the FDA to improve or stabilize symptoms of AD (Alzheimer's Disease Medications Fact Sheet: (July 2004) U.S. Department of Health and Human Services), including ARICEPT® (donepezil), EXELON® (rivastigmine), REMINYL® or RAZADYNE® (galantamine), COGNEX® (tacrine) and NAMENDA® (memantine). The effect achieved with many drugs currently in use is small (Tariot et al., JAMA 291: 317-24, 2004). Treatments for AD remain a largely unmet clinical need.

U.S. Patent Application No. 2005009885 (Jan. 13, 2005) (Mullan et al.) discloses a method for reducing β-amyloid deposition using nilvadipine, as wells as methods of diagnosing cerebral amyloidogenic diseases using nilvadipine. Nimodipine has been studied for the treatment of dementia. (Fritze et al., J. Neural Transm. 46: 439-453, 1995; and Forette et al., Lancet 352: 1347-1351, 1998).

There continues to be a need to identify compounds that can treat the inexorable progression of brain degeneration which is a hallmark of AD, wherein the treatment addresses β-amyloid production and the concomitant β-amyloid deposition, β-amyloid neurotoxicity (including abnormal hyperphosphorylation of tau), microglial-activated inflammation, and altered or over expression of APP which is seen in AD patients.

### SUMMARY

The present inventors have found polyhydroquinoline and dihydropyridine compounds that inhibit β-amyloid production, particularly, Aβ1-40 and Aβ1-42 production individually and total production of Aβ1-40+Aβ1-42. These compounds may be used in methods of treating, preventing, managing, slowing the progression of, delaying the onset of and/or ameliorating one or more symptoms of a disease associated with accumulation of β-amyloid, such as, but not limited to Alzheimer's Disease, or AD, in a subject in need thereof. Polyhydroquinoline compounds useful in the methods of the invention are listed in Table 1. Dihydropyridine and related compounds useful in the methods of the invention are listed in Tables 2 and 3.

Tables 1, 2, and 3 provide a list of polyhydroquinoline, dihydropyridine, and related compounds and report the activity of each compound to alter the levels of β-amyloid peptides, particularly Aβ1-40 and Aβ1-42, in cells that overexpress APP, e.g., Chinese Hamster Ovary (CHO) cells that overexpress APP751 (e.g., as described in Example 1, infra.) The compounds used in the methods of the invention reduce Aβ1-40 and/or Aβ1-42 production, and optionally both, and reduce one of Aβ1-40 and/or Aβ1-42 (or both) by at least 1%, 2%, 5%, 15%, 20%, 25%, 30%, 40%, 50%, 60%, 70%, 80%, 90%, 95%, or even at least 99%. An entry of “0” indicates no detectable amount of Aβ1-40 or Aβ1-42 according to the assay conditions. The data in tables 1, 2, and 3 may be rounded to the nearest 0.1%. The β-amyloid concentrations may be measured intracellularly or extracellularly (e.g., in the culture medium). The compounds may be tested at a range of concentrations, for example, from about 1 mM to 10 mM, about 500 nM to 50 μM, or about 5 μM to 30 μM.

The invention provides methods of treating, preventing managing, slowing the progression of, delaying the onset of, and/or ameliorating one or more symptoms of a disease or disorder associated with increased accumulation of β-amyloid, preferably cerebral accumulation of β-amyloid, such as, but not limited to AD, by administering an effective amount of a compound in Tables 1, 2, and 3, or a pharmaceutically acceptable salt, prodrug or derivative thereof, to a non-human animal or human subject. The invention provides pharmaceutical compositions comprising a therapeutically effective amount of a compound listed in Tables 1, 2, and 3, or pharmaceutically acceptable salt, prodrug or derivative thereof, and a pharmaceutically acceptable carrier, for use in the methods of the invention described herein, as well as unit dosage forms thereof. Also provided is the use of a compound disclosed in Tables 1, 2, and 3, or a pharmaceutically acceptable salt, prodrug or derivative thereof, in the manufacture of a medicament for the treatment of a disease associated with cerebral accumulation of β-amyloid.

In specific embodiments, the disease associated with cerebral accumulation of Alzheimer's amyloid is AD. In other embodiments, the disease is cerebral amyloid angiopathy, hereditary cerebral hemorrhage with amyloidosis Dutch-type, other forms of familial Alzheimer's disease and familial cerebral Alzheimer's amyloid angiopathy, transmissible spongiform encephalopathy, scrapie (and any other prion-based diseases), traumatic brain injury and Gerstmann-Straussler-Scheinker syndrome.

The method may, in one embodiment, include one or more of reducing β-amyloid production, β-amyloid deposition, β-amyloid neurotoxicity (including abnormal hyperphosphorylation of tau) and microgliosis. Because most diseases having cerebral accumulation of Alzheimer's amyloid, such as AD, are chronic, progressive, intractable brain dementias, it is contemplated that the duration of treatment with at least one of the active agents can optionally last for up to the lifetime of the animal or human.

Also provided is a method for treating head injury, and, optionally, reducing the risk of β-amyloid production, β-amyloid deposition, β-amyloid neurotoxicity (including abnormal hyperphosphorylation of tau) or microgliosis, in animals or humans suffering from traumatic brain injury, the method comprising administering to the animal or human a therapeutically effective amount in unit dosage form of a compound listed in Tables 1, 2, or 3, or a pharmaceutically acceptable salt, prodrug, or derivative thereof. In particular embodiments, the compound is administered immediately after the head injury, e.g., no more than 1 hour, 2 hours, 3 hours, 4 hours, 5 hours, 6 hours, 7 hours, 8 hours, 10 hours, 12 hours, 15 hours, 18 hours, or 24 hours after the injury has occurred, then, optionally, continuing treatment with the compound for a prescribed period of time thereafter. In one embodiment, the compound reduces the risk of β-amyloid production, Aβ deposition, β-amyloid neurotoxicity and/or microgliosis.

In specific embodiments, the invention provides methods of delaying the onset of or slowing the progression of a disease or disorder associated with increased β-amyloid accumulation. For example, the methods may slow the mental deterioration and loss of cognitive function that occurs in many such diseases, such as AD. In specific embodiments, human subjects suffering from AD retain mental function (e.g., can live unassisted) for at least 6 months, 1 year, 18 months, 2 years, 3 years, 4 years, 5 years, 7 years, 10 years, 12 years, 15 years, 18 years or even at least 20 years longer, on average, than comparable patients not subject to a method of the invention or for at least that period of time after diagnosis. In certain embodiments, the subject is elderly, specifically, at least 65, 75 or 85 years old.

In other embodiments, the invention provides methods of delaying the onset of diseases or disorders associated with accumulation of β-amyloid in subjects exhibiting early signs of such a disease or disorder or having a predisposition for such a disease or disorder. For example, subjects may exhibit early signs of memory loss or other loss of cognitive function, or behavioral or physical changes associated with early AD, or other disease or disorder associated with cerebral accumulation of β-amyloid. In these early stage subjects, methods of the invention may show the progression of the disease and delay onset of later stages of the disease by at least, on average, 6 months, 1 year, 18 months, 2 years, 3 years, 4 years, 5 years, 7 years, 10 years, 12 years, 15 years, 18 years or even at least 20 years. Subjects predisposed to a disease or disorder associated with accumulation of β-amyloid may be over the age of 65, 70, 75, 80 or 85, have a family history of such a disease or disorder, particularly, early onset AD (e.g., have at least a first degree relative or at least a second degree relative having been diagnosed with such a disease or disorder), have the ApoE epsilon 4 genotype, and/or have a history of head injury (particularly repeated head injury). In subjects having such a predisposition, methods of the invention may delay the onset of the disease or disorder by, on average, 1 year, 2 years, 5 years, 10 years, 15 years or 20 years or reduce risk of developing such a disease or disorder by at least 10%, 20%, 30%, 40%, 50%, 60%, 70%, 80% or 90%.

In another embodiment, a diagnostic method for a disease associated with cerebral accumulation of Alzheimer's amyloid in an animal or human is provided, comprising: taking a first measurement of plasma, urine, serum, whole blood, or cerebral spinal fluid (CSF) concentration of β-amyloid in the peripheral circulation of the animal or human; administering a diagnostically effective amount in unit dosage form of at least one active agent selected from the compounds listed in Tables 1, 2, or 3, or pharmaceutically acceptable salt, prodrug or derivative thereof, to the animal or human; taking a second measurement of plasma, serum, whole blood, urine or CSF concentration of β-amyloid in the peripheral circulation of the animal or human; and calculating the difference between the first measurement and the second measurement, wherein a change in the plasma, serum, whole blood, urine or CSF concentration of β-amyloid in the second measurement compared to the first measurement, in particular, an increase in concentration, indicates a possible diagnosis of a disease associated with cerebral accumulation of Alzheimer's amyloid in the animal or human.

### DETAILED DESCRIPTION OF THE INVENTION

The invention provides methods of treating, preventing, managing, delaying the onset of, slowing the progression of, and ameliorating one or more symptoms of a disease or disorder associated with β-amyloid accumulation, particularly, cerebral β-amyloid accumulation, by administration to a subject in need thereof an effective amount of a pharmaceutical composition comprising a compound selected from the compounds listed in Tables 1, 2, and 3, supra, and pharmaceutically acceptable salts, prodrugs and derivatives thereof. The invention further provides pharmaceutical compositions comprising a compound selected from the compounds listed in Tables 1, 2, and 3, and pharmaceutically acceptable salts, prodrugs and derivatives thereof; and a pharmaceutically acceptable carrier, and methods of diagnosis using the compounds listed in Tables 1, 2, and 3, and pharmaceutically acceptable salts, prodrugs and derivatives thereof.

### DEFINITIONS

As used herein, the term “Alzheimer's amyloid” is defined as a β-amyloid amino acid fragment that is for example proteolytically derived from amyloid precursor protein (APP). A β-amyloid amino acid fragment may include, for example, about 5 to 47 consecutive amino acids of the β-amyloid sequence. As used herein, the terms “β-amyloid,” “β-amyloid protein” and “Aβ” are used interchangeably with Alzheimer's amyloid that accumulates cerebrally in an animal or human.

As used herein the phrase a cell that “overexpresses APP or fragment thereof” refers to a cell that overexpresses an amyloid precursor protein, or fragment thereof, that in one preferred embodiment, includes a β-amyloid sequence and β- and γ-secretase cleavage sites. The cell that overexpresses APP or a fragment thereof preferably expresses an APP or fragment thereof that produces β-amyloid in the cell in which it is expressed.

As used herein, the term “amyloidogenic disease” includes a disease associated with cerebral accumulation of Alzheimer's amyloid.

The terms “host,” “subject,” and “patient,” as used herein, unless otherwise specified, include mammals (e.g., cats, dogs, horses, mice, cows, sheep, etc.), humans, or other organisms in need of treatment, all of which can be treated or diagnosed using the methods described herein.

The term “elderly,” as used herein, means a human who is 65 years or older.

As used herein, the phrase “in combination” refers to the use of more than one therapeutic agent. The use of the term “in combination” does not restrict the order in which therapeutic agents are administered to a subject with a disease or disorder. A first therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent (different from the first therapeutic agent) to a subject with a disease or disorder.

The terms “treatment,” “treat” and “treating,” as used herein, include any manner in which one or more of the symptoms of a disease or disorder are ameliorated or otherwise beneficially altered.

The terms “prevent,” “preventing” and “prevention,” as used herein, refer to the prevention of the onset of one or more symptoms of a disease or disorder associated with accumulation of β-amyloid in a subject resulting from the administration of a prophylactic or therapeutic agent.

As used herein, the term “therapeutically effective amount” refers to that amount of a therapeutic agent sufficient to result in amelioration of one or more symptoms of a disorder.

The term “pharmaceutically acceptable salt,” as used herein, unless otherwise specified, includes those salts which are, within the scope of sound medical judgment, suitable for use in contact with the tissues of hosts without undue toxicity, irritation, allergic response and the like, and are commensurate with a reasonable benefit/risk ratio and effective for their intended use. The salts can be prepared in situ during the final isolation and purification of one or more compounds of the composition, or separately by reacting the free base function with a suitable organic acid. Non-pharmaceutically acceptable acids and bases also find use herein, as for example, in the synthesis and/or purification of the compounds of interest. Nonlimiting examples of such salts are (a) acid addition salts formed with inorganic salts (for example hydrochloric acid, hydrobromic acid, sulfuric acid, phosphoric acid, nitric acid, and the like), and salts formed with organic salts such as acetic acid, oxalic acid, tartaric acid, succinic acid, ascorbic acid, benzoic acid, tannic acid, and the like; (b) base addition salts formed with metal cations such as zinc, calcium, magnesium, aluminum, copper, nickel and the like; (c) combinations of (a) and (b).

The term “pharmaceutically acceptable prodrugs,” as used herein, unless otherwise specified, includes those prodrugs of one or more compounds of the composition which are, within the scope of sound medical judgment, suitable for use in contact with the tissues of hosts without undue toxicity, irritation, allergic response and the like, are commensurate with a reasonable benefit/risk ratio, and are effective for their intended use. Pharmaceutically acceptable prodrugs also include zwitterionic forms, where possible, of one or more compounds of the composition. The term “prodrug” includes compounds that are transformed in vivo to yield the parent compound, for example by hydrolysis in blood or in the digestive system.

As used herein, the term “pharmaceutically acceptable derivative” means any salt, ester, or salt of such ester or any other compound which upon administration to an individual is capable of providing (directly or indirectly) a compound of the invention. The phrase includes active metabolites or residues of the compounds according to the invention.

The term “enantiomerically enriched,” as used herein, refers to a compound that is a mixture of enantiomers in which one enantiomer is present in excess, and preferably present to the extent of 95% or more, and more preferably 98% or more, including 100%.

By the term “about” is meant within ±10% of the stated amount, or within experimental error of the measuring technique.

Methods of Treatment

The invention provides methods for treating an animal or human afflicted with a disease associated with cerebral accumulation of Alzheimer's amyloid, such as Alzheimer's disease (AD), comprising administering a therapeutically effective amount of a compound disclosed herein, or a pharmaceutically acceptable salt, prodrug or derivative thereof. Administration of the compound in one embodiment results in reducing one or more of β-amyloid production, β-amyloid deposition, β-amyloid neurotoxicity (including abnormal hyperphosphorylation of tau) or microgliosis, or combination thereof. In one embodiment, the compound is characterized in that it reduces β-amyloid production, for example, by at least about 5%, 10%, 15%, 20%, 25%, 30%, 50%, 70%, 80%, 90%, 95% or more in cultured cells that overexpress APP or a fragment thereof, as measured, for example, in a culture medium comprising the cells or as measured intracellularly.

As used herein, reference to a compound that reduces β-amyloid production, refers to a compound that reduces β-amyloid production, either Aβ1-40 or Aβ1-42, or both, in cells that overexpress APP or a fragment thereof, and the cells may be, for example, Chinese hamster ovary (CHO) cells that overexpress APP, for example, 7W WT APP751 CHO cells; 7W (wt APP₇₅₁) cells; 7W_{ΔC} cells; 7W_{SW} cells; or 7W_{VF} cells. In one embodiment, the compound and method according to the invention achieve a greater relative reduction in Aβ1-42 compared to reduction in Aβ1-40. In other words, where Aβ1-42 is more pathogenic than Aβ1-40, compounds and methods according to one embodiment of the invention selectively reduce production of Aβ1-42. For example, Aβ1-42 may be selectively reduced by at least about 5%, 10%, 15%, 20%, 25%, 30%, 50%, 70%, 80%, 90%, 95% or more compared to the reduction in Aβ1-40 in cultured cells that overexpress APP or a fragment thereof, as measured, for example, in a culture medium comprising the cells or as measured intracellularly.

It is noted that wherever the embodiments disclosed herein refer to a reduction in β-amyloid in cells that overexpress APP, alternatively, an increase in αCTF (α C-terminal APP fragment, also known as CTF-α) and/or APPSα soluble fragment can be measured for example, in the cell culture or intracellularly. Alpha-CTF and APPSα soluble fragment are produced in increased amounts from APP when the production of β-amyloid decreases.

It is further noted that wherever the embodiments disclosed herein refer to a reduction in β-amyloid in cells that overexpress APP, alternatively, a decrease in β CTF (β C-terminal APP fragment, also known as CTF-β) or APPSβ soluble fragment can be measured, e.g., in the cell culture media or intracellularly, as they are produced in decreased amounts from APP as the compound causes the production of β-amyloid to decrease.

In a further embodiment, a method is provided for treating animals or humans suffering from traumatic brain injury (TBI). In one embodiment, β-amyloid production, β-amyloid deposition, β-amyloid neurotoxicity (including abnormal hyperphosphorylation of tau) and/or microgliosis is reduced. The method includes administering to the animal or human, for example, immediately (30 minutes, 1 hour, 2 hours, 3 hours, 4 hours, 5 hours, 6 hours, 8 hours, 10 hours, 12 hours, 15 hours, 18 hours, 24 hours, 36 hours or 48 hours) after the TBI, a therapeutically effective amount of a compound selected from the compounds listed in Tables 1, 2, and 3, and pharmaceutically acceptable salts, prodrugs and derivatives thereof. The method may include continuing treatment with the compound for a prescribed period of time thereafter. It has been shown that TBI increases the susceptibility to AD, and thus it is believed, without being bound by the theory, that TBI accelerates brain β-amyloid accumulation and oxidative stress, which may work synergistically to promote the onset or drive the progression of AD. Treatment with the compound of animals or humans suffering from one or more TBIs can continue, for example, for about one hour, 24 hours, a week, two weeks, 1-6 months, one year, two years or three years. Such treatment reduces the risk of developing AD by 10%, 20%, 30%, 40%, 50%, 60%, 70% or even 80% or delays the onset of AD by, on average, 1 year, 2 years, 5 years, 10 years, 15 years, 20 years, or 25 years or reduce the risk of developing the disease or disorder by 10%, 20%, 30%, 40%, 50%, 60%, 70%, 80%, or 90%.

Amyloidogenic diseases which can be treated according to the methods of the present invention can include, without limitation, Alzheimer's disease, cerebral amyloid angiopathy, hereditary cerebral hemorrhage with amyloidosis Dutch-type, or other forms of familial AD and familial cerebral Alzheimer's amyloid angiopathy.

In specific embodiments, the invention provides methods of delaying the onset of or slowing the progression of a disease or disorder associated with increased β-amyloid accumulation. For example, the methods may slow the mental deterioration and loss of cognitive function, adverse changes in behavior and/or physical deterioration that occurs in many such diseases, such as AD. In specific embodiments, human or animal subjects suffering from an amyloidogenic disease, such as AD, retain mental function (e.g., can live unassisted) for at least 6 months, 1 year, 18 months, 2 years, 3 years, 4 years, 5 years, 7 years, 10 years, 12 years, 15 years, 18 years or even at least 20 years longer, on average, than comparable patients not subject to a method of the invention. In certain embodiments, the subject is at least 65, 75 or 85 years old.

In other embodiments, the invention provides methods of delaying the onset of diseases or disorders associated with accumulation of β-amyloid in subjects exhibiting early signs of such a disease or disorder or having a predisposition to such a disease or disorder. For example, subjects may exhibit early signs of memory loss or other loss of cognitive function, adverse behavioral changes, or other signs of physical impairment associated with a disease or disorder characterized by accumulation of β-amyloid, particularly AD. Subjects predisposed to a disease or disorder associated with accumulation of β-amyloid may be over the age of 65, 70, 75, 80 or 85, have a family history (e.g., having at least a first degree relative or at least a second degree relative with such a disease or disorder) of such a disease or disorder, particularly, early onset AD, have the ApoE epsilon 4 genotype, and/or have a history of head injury (particularly repeated head injury). In subjects having such a predisposition, methods of the invention may delay the onset of the disease or disorder by, on average, 1 year, 2 years, 5 years, 10 years, 15 years or 20 years or reduce the risk of developing the disease or disorder by 10%, 20%, 30%, 40%, 50%, 60%, 70%, 80% or 90%.

The compounds of the invention may be administered in combination with other therapeutic agents that are useful for the treatment, prevention, management, delaying the onset, slowing the progression or amelioration of one or more symptoms of a disease or disorder associated with accumulation of β-amyloid, either when administered alone or in combination with a compound of the invention. Such therapeutic agents useful for such combination therapy include, but are not limited to ARICEPT® (donepezil), EXELON® (rivastigmine), REMINYL® or RAZADYNE® (galantamine), COGNEX® (tacrine) and NAMENDA® (memantine), NSAIDS such as ibuprofen, etc., and agents that have efficacy in the treatment of depression, continency and other symptoms of diseases and disorders associated with accumulation of β-amyloid. The effects of the combination may be additive or, preferably, are synergistic.

Exemplary dosages of compound that can be administered include 0.001-1.0 mg/kg body weight. An exemplary dose of compound is about 1 to 50 mg/kg body weight per day, 1 to 20 mg/kg body weight per day, or 0.1 to about 100 mg per kilogram body weight of the recipient per day. Lower doses may be preferable, for example doses of 0.5-100 mg, 0.5-50 mg, 0.5-10 mg, or 0.5-5 mg per kilogram body weight per day, or e.g., 0.01-0.5 mg per kilogram body weight per day. The effective dosage range can be calculated based on the activity of the compound and other factors known in the art of pharmacology.

The compound is conveniently administered in any suitable dosage form, including but not limited to one containing 1 to 3000 mg, or 10 to 1000 mg of active ingredient per unit dosage form. An oral dosage of 50-1000 mg is possible. Lower doses may be preferable, for example from 10-100 or 1-50 mg, or 0.1-50 mg, or 0.1-20 mg or 0.01-10.0 mg. Furthermore, lower doses may be utilized in the case of administration by a non-oral route, as, for example, by injection or inhalation.

In another embodiment, the dosage can range from about 0.05 mg to 20 mg per day, from between about 2 mg to 15 mg per day, about 4 mg to 12 mg per day, and or about 8 mg per day.

In another embodiment, the dosage ranges, e.g. from about one day to twelve months, from about one week to six months, or from about two weeks to four weeks.

Because most diseases having cerebral accumulation of Alzheimer's amyloid, such as AD, are chronic, progressive, intractable brain dementias, it is contemplated that the duration of treatment with compounds disclosed herein can last for up to the lifetime of the animal or human.

In another embodiment of the present invention, a method is provided for increasing cerebral blood flow in an animal or human to improve cognition or slow the progress of an impairment of cognition by administering a compound according to Formulas I or II or selected from the compounds listed in Tables 1, 2, and 3, and pharmaceutically acceptable salts, prodrugs and derivatives thereof. Impairment of cognition includes MCI (Mild Cognitive Impairment). A condition of MCI may exist irrespective of a patient's status with respect to a diagnosis related to Alzheimer's amyloid. Without wishing to be bound by theory, the administration of compounds according to the invention may yield increased cerebral blood flow compared to baseline cerebral blood flow, and such increased blood flow may reduce β-amyloid deposition or provide other clinical benefit. Diseases associated with decreased cerebral blood flow can include without limitation stroke, such as ischemic stroke, ischemia, depression, including subcortical ischemic depression, giant cell arteritis, temporal arteritis, cerebral vasospasm, infarction, obstruction of a cerebral blood vessel, hemorrhage, such as subarachnoid hemorrhage, or any other indication related to restricted cerebral blood flow.

Methods of Diagnosis

In a further embodiment, a method is provided for diagnosing or determining the risk for developing a disease associated with cerebral accumulation of Alzheimer's amyloid, such as AD, in an animal or human, by taking a first measurement of β-amyloid concentration from a peripheral body fluid such as plasma, serum, whole blood, urine or cerebral spinal fluid (CSF) of the animal or human. Subsequently, the method includes administering to the animal or human a diagnostically effective amount of a compound selected from the compounds listed in Tables 1, 2, and 3, and pharmaceutically acceptable salts, prodrugs and derivatives thereof. In one embodiment, the compound decreases β-amyloid production, for example, by at least about 5%, 10%, 15%, 20%, 25%, 30%, 50%, or more, as measured, for example, in the medium of cultured cells which overexpress APP or a fragment thereof, or as measured intracellularly. A second (selected endpoint) measurement of β-amyloid concentration is taken from plasma, serum, whole blood, urine or CSF of the animal or human at a later time, and the difference between the first measurement and the second measurement is determined. A change in the concentration of β-amyloid in plasma, serum, whole blood, urine or CSF in the second measurement compared to the first measurement indicates a risk of developing or a possible diagnosis of a disease associated with cerebral accumulation of Alzheimer's amyloid in the animal or human. In particular, an increase in peripheral β-amyloid indicates the presence of an accumulation of cerebral β-amyloid, and therefore the risk of disease or the presence of the disease.

It is believed, without being bound by any theory, that the compounds can cause an increase in β-amyloid concentration in plasma, urine, serum, whole blood or CSF by facilitating the clearance of already produced β-amyloid from the central nervous system into the periphery, thus increasing β-amyloid concentration in the peripheral fluid being assayed.

The duration of time of administration of the compound after the first peripheral body fluid measurement, up until the second (selected endpoint) peripheral body fluid measurement, is, e.g., any suitable time period, e.g. about 1-12 hours, about 1-7 days, about 1-4 weeks; about 2-6 months, or more. The time length can be adjusted as needed depending, for example, on the progression of the disease, and the patient. A suitable periodic (e.g., daily) dosage of the compound is administered, e.g. orally or intravenously, and the β-amyloid levels in the individual can be monitored periodically up until the endpoint. In one preferred embodiment, the compound is administered daily for about 3 days to 4 weeks from the start of administration to the endpoint measurement. The change in concentration indicative of the risk or presence of a disease associated with β-amyloid accumulation is, e.g. about 10-20% or more between the first and endpoint measurements.

Exemplary dosages of compound that can be administered include 0.001-1.0 mg/kg body weight, for example daily. An exemplary dose of compound is about 1 to 50 mg/kg body weight per day, 1 to 20 mg/kg body weight per day, or 0.1 to about 100 mg per kilogram body weight of the recipient per day. Lower doses may be preferable, for example doses of 0.5-100 mg, 0.5-50 mg, 0.5-10 mg, or 0.5-5 mg per kilogram body weight per day, or e.g., 0.01-0.5 mg per kilogram body weight per day. The effective dosage range can be calculated based on the activity of the compound and other factors known in the art of pharmacology.

The compound is conveniently administered in any suitable dosage form, including but not limited to, one containing 1 to 3000 mg, or 10 to 1000 mg of active ingredient per unit dosage form. An oral dosage of 50-1000 mg is possible. Lower doses may be preferable, for example from 10-100 or 1-50 mg, or 0.1-50 mg, or 0.1-20 mg or 0.01-10.0 mg. Furthermore, lower doses may be utilized in the case of administration by a non-oral route, as, for example, by injection or inhalation.

In one embodiment, the invention comprises a method for diagnosing a disease associated with cerebral accumulation of Alzheimer's amyloid in an animal or human subject, comprising: taking a first measurement of plasma, urine, serum, whole blood, or cerebral spinal fluid (CSF) concentration of β-amyloid or fragment thereof in the peripheral circulation of the animal or human subject; (a) administering to the animal or human subject a diagnostically effective amount of a compound selected from the compounds listed in Tables 1, 2, and 3, and derivatives, salts and prodrugs thereof; (b) taking a second measurement of plasma, serum, whole blood, urine or CSF concentration of β-amyloid in the peripheral circulation of the animal or human; and (c) calculating the difference between the first measurement and the second measurement; wherein a change in the plasma, serum, whole blood, urine or CSF concentration of β-amyloid in the second measurement compared to the first measurement indicates a possible diagnosis of a disease associated with cerebral accumulation of Alzheimer's amyloid in the animal or human subject.

Compounds

A variety of compounds are provided herein in Tables 1, 2, and 3, which can be used in methods described herein, including the treatment or diagnosis of diseases associated with cerebral accumulation of Alzheimer's amyloid. Compounds useful in the methods and compositions described herein are in one embodiment available from commercially sources or can be synthesized using methods routine in the art.

Optionally, the compounds listed in Tables 1, 2, and 3 can be represented by the following formulas.

Polyhydroquinoline compounds according to Formula I:

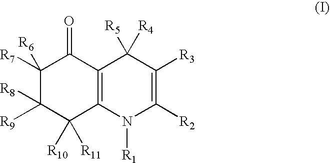

wherein
R1 through R11 may be the same or different from each other and each represent hydrogen atom, alkyl, alkenyl, alkynyl, alkoxy, carboxy, carboxamido, amino, aminocarboxy, cyano, halogen, aryl, alkaryl, alkenaryl, azido, heteroaryl, cycloalkyl, heterocycloalkyl, carbamoyl, methyl thiocarbamoyl, alkyl ester, aryl ester, alkyloxyalkylester, alkylthioalkylester, and thiolalkyl groups, in which each group is optionally further substituted. In addition, adjacent R groups (i.e. R1 and R2, R2 and R3, R7 and R8, etc.) may together form cyclo, heterocyclo, aryl, or heteroaryl groups.

Representative aryl and heteroaryl groups include phenyl, benzyl, chromene, 1-naphthyl, 2-naphthyl, thiophene-3-yl, thiophene-2-yl, furan-3-yl, furan-2-yl, pyrrolo, pyridine-4-yl, pyridine-3-yl, pyridine-2-yl, pyridine-4-ylmethyl, pyridine-3-ylmethyl, pyridine-2-ylmethyl, 1-naphthyl, 2-naphthyl, thiophene-3-yl, thiophene-2-yl, furan-3-yl, furan-2-yl, pyridine-4-yl, pyridine-3-yl, pyridine-2-yl, carbazolyl, indole-2-yl, and indole-3-yl groups.

In one embodiment, R4 is an optionally substituted aromatic or heteroaromatic ring, and R5 is hydrogen. In another embodiment, R4 is an unsubstituted aromatic or heteroaromatic ring, and R5 is hydrogen.

In one embodiment, R4 is a nonaromatic substituent and R5 is hydrogen.

In one embodiment, R2 and R3 together form a ring which may optionally be fused with one or more additional rings.

In one embodiment, R1, R6, R7, R8, R9, R10, and R11 are hydrogen. Alternatively, R8 and R9 are hydrogen, methyl, optionally substituted phenyl, or thienyl.

In one embodiment, R2 is selected from lower alkyl, including methyl, amino, and thiol, and aryl, such as phenyl group, each group being optionally substituted. R2 may optionally form a ring together with R1 or R3. In one embodiment R1 and R2 join together with the main ring to form a 1,3-thiazolidino[3,2-a]pyridine ring.

In one embodiment, R3 is selected from a cyano group or carboxylate esters, such as alkyl esters, including methyl, ethyl, propyl, butyl, pentyl, hexyl, branched alkyls; cycloalkyl esters, including cyclopentyl, cyclohexyl, and cycloheptyl; aryl esters, including phenyl, benzyl; allyl esters; and optional substitutions, such as methoxyethyl esters, ethoxyethyl esters, phenoxyethyl esters, phenylethylesters, methoxybenzyl esters; aryl-substituted amides, and the like.

In one embodiment, R4 is an aromatic group selected from phenyl, pyridinyl, furyl, pyrrolo, and thienyl, optionally unsubstituted or optionally with one or more substitutions, including alkoxy, nitro, halogen, acetoxy, trifluoromethyl, phenoxy, dialkylamino, 1,3-dioxalenyl, and alkyl substituents.

Dihydropyridine compounds according to Formula II:

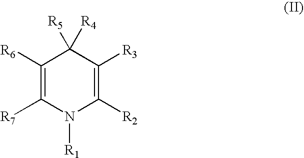

wherein
R1 through R7 may be the same or different from each other and each represent hydrogen atom, alkyl, alkenyl, alkynyl, alkoxy, carboxy, carboxamido, amino, aminocarboxy, cyano, halogen, aryl, alkaryl, alkenaryl, azido, sulfonyl, heteroaryl, cycloalkyl, heterocycloalkyl, carbamoyl, methylthiocarbamoyl, alkyl ester, and aryl ester, in which each group is optionally further substituted. In addition, adjacent R groups (i.e. R1 and R2, R2 and R3, R6 and R7, R1 and R7, etc.) may together form cyclo, heterocyclo, aryl, or heteroaryl groups.

Representative aryl and heteroaryl groups include phenyl, benzyl, chromene, 1-naphthyl, 2-naphthyl, thiophene-3-yl, thiophene-2-yl, furan-3-yl, furan-2-yl, pyrrolo, pyridine-4-yl, pyridine-3-yl, pyridine-2-yl, pyridine-4-ylmethyl, pyridine-3-ylmethyl, pyridine-2-ylmethyl, 1-naphthyl, 2-naphthyl, thiophene-3-yl, thiophene-2-yl, furan-3-yl, furan-2-yl, pyridine-4-yl, pyridine-3-yl, pyridine-2-yl, carbazolyl, indole-2-yl, and indole-3-yl groups.

In one embodiment, R1 is hydrogen. In one embodiment, R1 is a substituted or unsubstituted phenyl, benzyl or thienyl group. In one embodiment, R1 is an adamantyl group. In one embodiment, R1 is an alkyl group such as methyl. In embodiment, R1 is a 1-phenylethyl group.

In one embodiment, R2 or R7 are independently hydrogen, methyl, ethyl, propyl, amino, thiol, cyano, thioether, phenyl, thioacetamide, thioacetate, optionally substituted, for example at the nitrogen of the thioacetamide with an optionally substituted phenyl ring.

In one embodiment R1 and (R2 or R7) join together with the dihydropyridine ring to form a 1,3-thiazolidino[3,2-a]pyridine ring.

In one embodiment, (R2 or R7) and (R3 or R6) together form a fused sulfur-containing heteroaromatic ring.

In one embodiment, R3 or R6 are independently hydrogen, cyano, sulfonyl, carboxylate esters, such as alkyl esters, including methyl, ethyl, propyl, butyl, pentyl, hexyl, branched alkyls, such as t-butyl; cycloalkyl esters, including cyclopentyl, cyclohexyl, and cycloheptyl; aryl esters, including phenyl, benzyl; allyl esters; and optional substitutions, such as methoxyethyl esters, ethoxyethyl esters, phenoxyethyl esters, phenylethylesters, methoxybenzyl esters; aryl-substituted amides, phenylcarbamoyl, and the like.

In one embodiment, R4 is an optionally substituted aromatic or heteroaromatic ring, and R5 is hydrogen. In one embodiment, R4 is an unsubstituted aromatic or heteroaromatic ring and R5 is hydrogen. In one embodiment, R4 is an aromatic group selected from phenyl, pyridinyl, furyl, pyrrolo, and thienyl, optionally with one or more substitutions, including alkoxy, nitro, halogen, acetoxy, trifluoromethyl, phenoxy, dialkylamino, 1,3-dioxalenyl, additional aromatic rings, and alkyl substituents.

In one embodiment, R4 is a nonaromatic substituent and R5 is hydrogen.

In one embodiment, R4 and R5 together form a double bond to an oxygen atom.

In one embodiment, R4 and R5 together form a double bond to a nitrogen atom which together with R3 forms a fused heteroaromatic ring.

It is not envisioned that every compound within Formulas I and 2 will have the same level of efficacy in the methods described herein, including the treatment or diagnosis of diseases associated with cerebral accumulation of Alzheimer's amyloid. Certain compounds within Formulas I and 2 are preferred embodiments, such as the compounds listed in Tables 1, 2, and 3. Preferred embodiments can be readily determined by one of ordinary skill in the art using the assays described herein. As a general guide, preferred embodiments according to the invention alter the levels of β-amyloid peptides, particularly Aβ1-40 and Aβ1-42, in cells that overexpress APP, e.g., Chinese Hamster Ovary (CHO) cells that overexpress APP751 (e.g., as described in Example 1, infra.). Guidance for the amount of alteration of production of β-amyloid peptides is provided above. In one embodiment of the invention, treated cells produce 90% or less Aβ1-40 and/or Aβ1-42 compared to control cells.

It is to be understood that the compounds disclosed herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the disclosure of a compound herein encompasses any racemic, optically active, polymorphic, or steroisomeric form, or mixtures thereof, which preferably possesses the useful properties described herein, it being well known in the art how to prepare optically active forms and how to determine activity using the standard tests described herein, or using other similar tests which are will known in the art. Examples of methods that can be used to obtain optical isomers of the compounds include the following:

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme

iv) enzymatic asymmetric synthesis, a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography, a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography, a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent; and

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Additional information on certain embodiments of the invention can be seen in the following tables. Data from Tables 1, 2, and 3 can be rounded to the nearest 0.1%. Values of zero indicate that the level of Aβ1-40 or Aβ1-42 was below the detection limit for the assay. Where nitrogen is shown with only two bonds, a third bond to an H atom is assumed.

TABLE 1
Polyhydroquinoline Compounds

| | | | Aβ1-40 | Aβ1-42 |
|---|---|---|---|---|
| | | | (% of | (% of |
| ID | IUPAC Name | Structure | control) | control) |
| ST013049 | phenylmethyl 4-[4- (diethylamino)phenyl]-2,7,7- trimethyl-5-oxo-1,4,6,7,8- pentahydroquinoline-3-carboxylate | 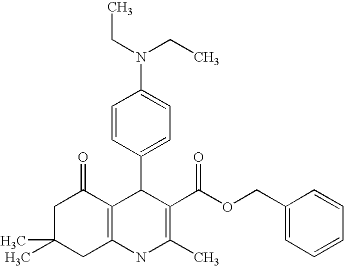 | 27.81322327 | 94.14198698 |
| ST013052 | phenylmethyl 2,7,7-trimethyl-4-(6- nitro(2H-benzo[d]1,3-dioxolan-5- yl))-5-oxo-1,4,6,7,8- pentahydroquinoline-3-carboxylate | 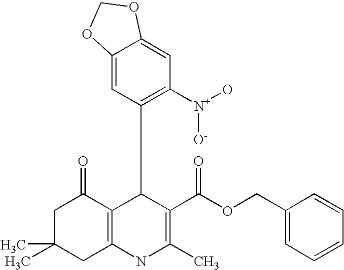 | 68.80972594 | 72.35237417 |
| ST013054 | pentyl 4-(4-chlorophenyl)-2,7,7- trimethyl-5-oxo-1,4,6,7,8- pentahydroquinoline-3-carboxylate | 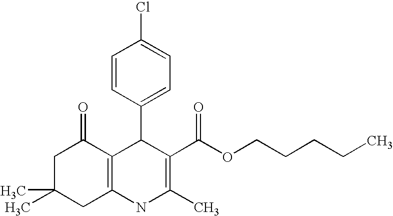 | 0 | 8.35898154 |
| ST013059 | methyl 2,7,7-trimethyl-5-oxo-4-(3- pyridyl)-1,4,6,7,8- pentahydroquinoline-3-carboxylate | 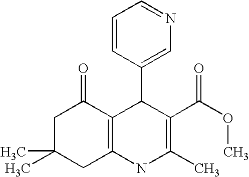 | 26.68792349 | 76.11236215 |
| ST013060 | ethyl 2,7,7-trimethyl-4-(6-nitro(2H- benzo[d]1,3-dioxolan-5 -yl))-5-oxo- 1,4,6,7,8-pentahydroquinoline-3- carboxylate | 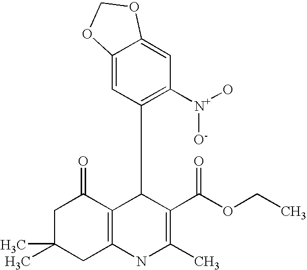 | 32.67242471 | 99.38781622 |
| ST013066 | ethyl 4-[4-(dimethylamino)phenyl]- 2-methyl-5-oxo-1,4,6,7,8- pentahydroquinoline-3-carboxylate | 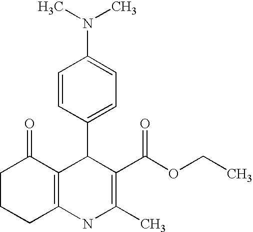 | 68.42450704 | 96.6696344 |
| ST013070 | ethyl 2-methyl-5-oxo-4-(3-pyridyl)- 1,4,6,7,8-pentahydroquinoline-3- carboxylate | 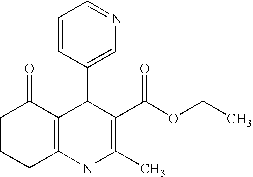 | 33.96416417 | 82.4138992 |
| ST013073 | methyl 4-(2,5-dimethoxyphenyl)-2- methyl-5-oxo-1,4,6,7,8- pentahydroquinoline-3-carboxylate | 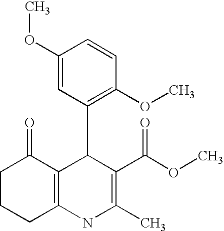 | 49.45771019 | 93.16887134 |
| ST013077 | methylethyl 2-methyl-5-oxo-4-(3- pyridyl)-1,4,6,7,8- pentahydroquinoline-3-carboxylate | 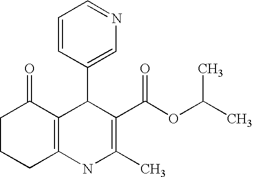 | 27.44265943 | 97.83491993 |
| ST013081 | cyclopentyl 4-(4-chlorophenyl)- 2,7,7-trimethyl-5-oxo-1,4,6,7,8- pentahydroquinoline-3-carboxylate | 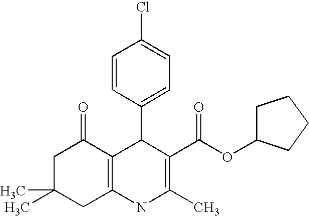 | 0 | 0.188081173 |
| ST013083 | cyclopentyl 4-[4- (diethylamino)phenyl]-2,7,7- trimethyl-5-oxo-1,4,6,7,8- pentahydroquinoline- 3-carboxylate | 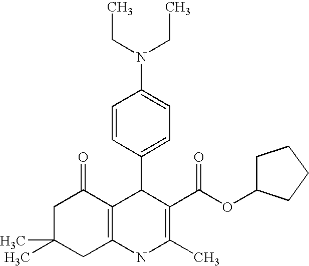 | 0 | 91.60943309 |
| ST013086 | cyclopentyl 4-(2,4- dimethoxyphenyl)-2,7,7-trimethyl- 5-oxo-1,4,6,7,8-pentahydroquinoline- 3-carboxylate | 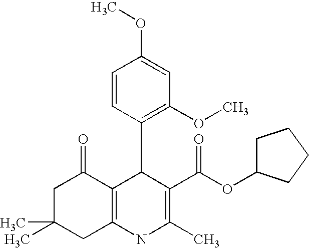 | 0 | 73.73926838 |
| ST013090 | methylethyl 2-methyl-5-oxo-4-(4- pyridyl)-1,4,6,7,8- pentahydroquinoline-3-carboxylate | 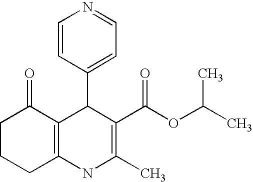 | 41.06768443 | 87.31382264 |
| ST013092 | ethyl 4-(3-iodophenyl)-2-methyl-5- oxo-1,4,6,7,8-pentahydroquinoline- 3-carboxylate | 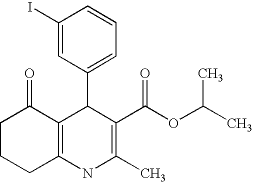 | 58.2381888 | 82.02138197 |
| ST013103 | cyclopentyl 4-(2-bromophenyl)- 2,7,7-trimethyl-5-oxo-1,4,6,7,8- pentahydroquinoline-3-carboxylate | 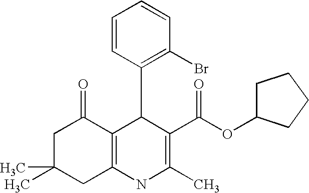 | 0 | 68.18024309 |
| ST013115 | methyl 4-(4-hydroxy-3- methoxyphenyl)-2-methyl-5-oxo-7- phenyl-1,4,6,7,8- pentahydroquinoline- 3-carboxylate | 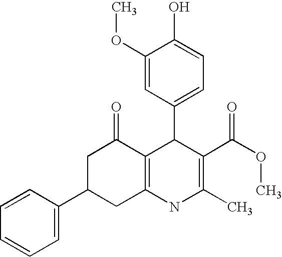 | 94.68997542 | 65.74282147 |
| ST013117 | methyl 4-(2,5-dimethoxyphenyl)-2- methyl-5-oxo-7-phenyl-1,4,6,7,8- pentahydroquinoline-3-carboxylate | 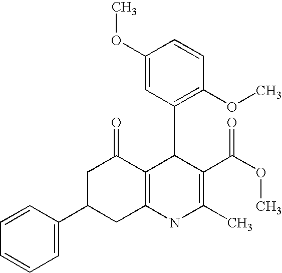 | 22.41281981 | 46.14772075 |
| ST013129 | methyl 4-[4-(diethylamino)phenyl]- 2-methyl-5-oxo-7-phenyl-1,4,6,7,8- pentahydroquinoline-3-carboxylate | 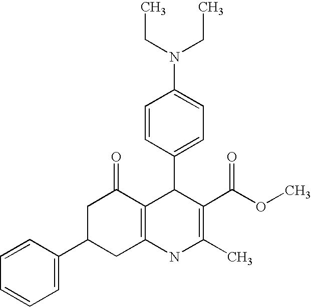 | 64.2775198 | 90.55369977 |
| ST013137 | cyclopentyl 4-(3-bromo-4- methoxyphenyl)-2-methyl-5-oxo- 1,4,6,7,8-pentahydroquinoline-3- carboxylate | 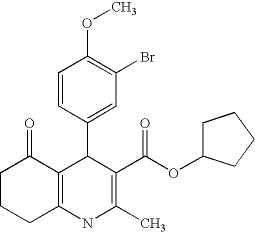 | 18.68706182 | 30.01990755 |
| ST013138 | phenylmethyl 4-(3,4- dimethoxyphenyl)-2-methyl-5-oxo- 1,4,6,7,8-pentahydroquinoline-3- carboxylate | 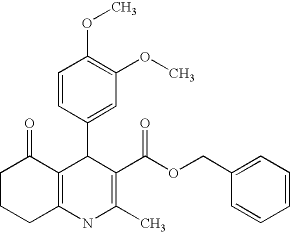 | 69.68041519 | 56.82154064 |
| ST013140 | methyl 4-(2,3-dimethoxyphenyl)-2- methyl-5-oxo-1,4,6,7,8- pentahydroquinoline-3-carboxylate | 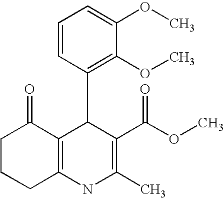 | 95.99016662 | 51.10571245 |
| ST013141 | methyl 4-(3-bromo-4-hydroxy-5- methoxyphenyl)-2-methyl-5-oxo- 1,4,6,7,8-pentahydroquinoline-3- carboxylate | 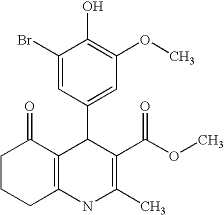 | 89.7204771 | 23.67513581 |
| ST013145 | methyl 4-(3-bromophenyl)-2,7,7- trimethyl-5-oxo-1,4,6,7,8- pentahydroquinoline-3-carboxylate | 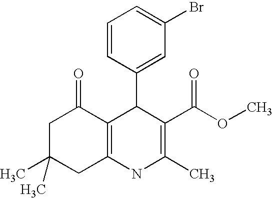 | 13.90330511 | 43.26618754 |
| ST013146 | ethyl 4-(3-methoxyphenyl)-2- methyl-5-oxo-1,4,6,7,8- pentahydroquinoline-3-carboxylate | 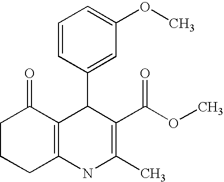 | 87.68642447 | 67.67756521 |
| ST013147 | methyl 2-methyl-5-oxo-4-(2,3,4- trimethoxyphenyl)-1,4,6,7,8- pentahydroquinoline-3-carboxylate | 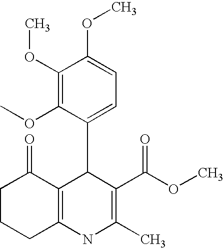 | 72.0021852 | 45.36289098 |
| ST013148 | ethyl 4-(3-hydroxyphenyl)-2- methyl-5-oxo-1,4,6,7,8- pentahydroquinoline-3-carboxylate | 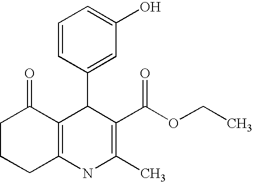 | 66.10762087 | 49.93352903 |
| ST013151 | methyl 4-(2,3-dichlorophenyl)- 2,7,7-trimethyl-5-oxo-1,4,6,7,8- pentahydroquinoline-3-carboxylate | 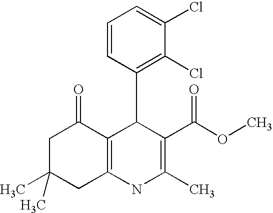 | 52.07684603 | 61.61082431 |
| ST013152 | pentyl 4-(3-chlorophenyl)-2- methyl-5-oxo-1,4,6,7,8- pentahydroquinoline-3-carboxylate | 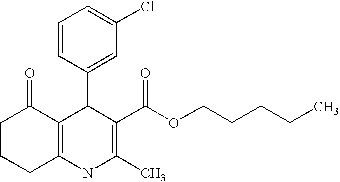 | 22.01766366 | 33.22536019 |
| ST013153 | methyl 4-(3-methoxyphenyl)-2- methyl-5-oxo-1,4,6,7,8- pentahydroquinoline-3-carboxylate | 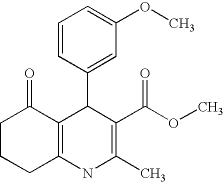 | 91.3939725 | 66.25906806 |
| ST013156 | cyclopentyl 4-(3-chlorophenyl)-2- methyl-5-oxo-1,4,6,7,8- pentahydroquinoline-3-carboxylate | 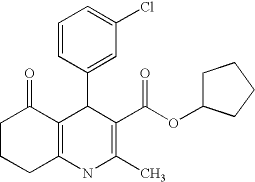 | 0 | 13.69436853 |
| ST013158 | ethyl 4-(3-ethoxy-4- hydroxyphenyl)-2-methyl-5-oxo- 1,4,6,7,8-pentahydroquinoline-3- carboxylate | 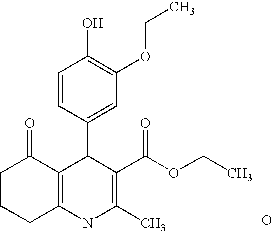 | 75.02868069 | 67.80645814 |
| ST013162 | ethyl 4-(3-fluorophenyl)-2-methyl- 5-oxo-1,4,6,7,8- pentahydroquinoline-3-carboxylate | 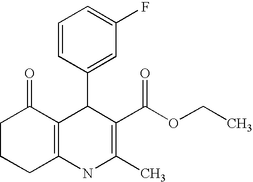 | 33.37521624 | 36.28032527 |
| ST013170 | pentyl 4-(3-bromo-4-hydroxy-5- methoxyphenyl)-2-methyl-5-oxo- 1,4,6,7,8-pentahydroquinoline-3- carboxylate | 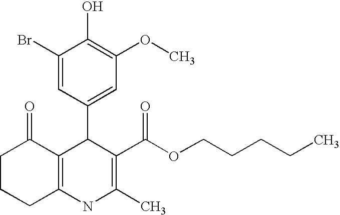 | 62.83346991 | 49.80328643 |
| ST013173 | methyl 4-[3-(ethoxycarbonyl)-2- methyl-5-oxo-4-1,4,6,7,8- pentahydroquinolyl]benzoate | 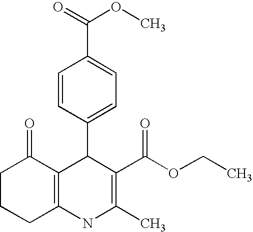 | 39.19147774 | 78.10034754 |
| ST013174 | methylethyl 4-(3-hydroxyphenyl)- 2-methyl-5-oxo-1,4,6,7,8- pentahydroquinoline-3-carboxylate | 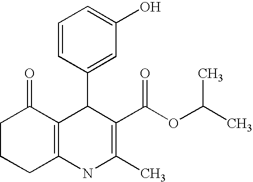 | 34.28571429 | 73.08566994 |
| ST013183 | prop-2-enyl 4-(3-methoxy-4- propoxyphenyl)-2-methyl-5-oxo- 1,4,6,7,8-pentahydroquinoline-3- carboxylate | 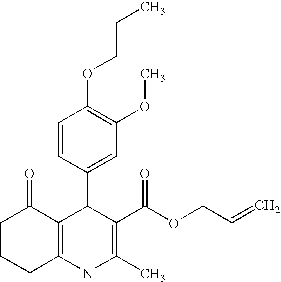 | 78.71437676 | 66.25906806 |
| ST013184 | ethyl 4-(2,3-dimethoxyphenyl)- 2,7,7-trimethyl-5-oxo-1,4,6,7,8- pentahydroquinoline-3-carboxylate | 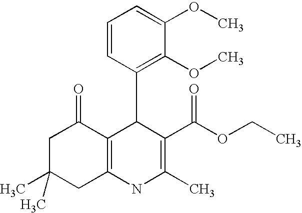 | 73.71392152 | 60.31784594 |
| ST013188 | cyclohexyl 4-(2,3-dichlorophenyl)- 2-methyl-5-oxo-1,4,6,7,8- pentahydroquinoline-3-carboxylate | 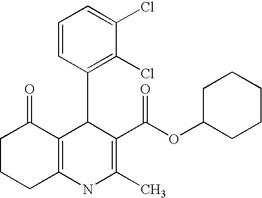 | 0 | 47.06279313 |
| ST013191 | methylethyl 4-(4-ethylphenyl)-2- methyl-5-oxo-1,4,6,7,8- pentahydroquinoline-3-carboxylate | 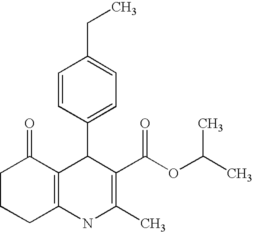 | 33.37521624 | 97.99507373 |
| ST013192 | 4-{2-methyl-3- [(methylethyl)oxycarbonyl]-5-oxo- 4-1,4,6,7,8-pentahydroquinolyl} phenyl acetate | 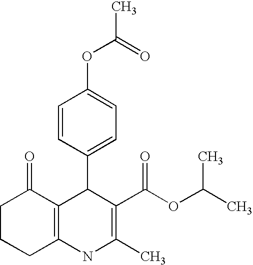 | 55.50213967 | 82.46786112 |
| ST013199 | prop-2-enyl 4-(3-methoxyphenyl)- 2-methyl-5-oxo-1,4,6,7,8- pentahydroquinoline-3-carboxylate | 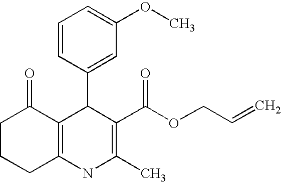 | 68.48577567 | 86.67952236 |
| ST013202 | propyl 4-(3-bromophenyl)-2,7,7- trimethyl-5-oxo-1,4,6,7,8- pentahydroquinoline-3-carboxylate | 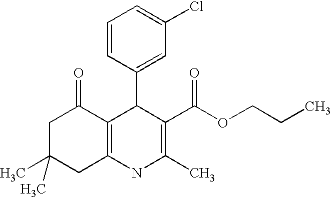 | 0 | 39.14929912 |
| ST013206 | propyl 4-(2,3-dichiorophenyl)- 2,7,7-trimethyl-5-oxo-1,4,6,7,8- pentahydroquinoline-3-carboxylate | 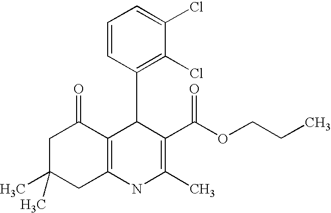 | 0 | 62.53695129 |
| ST013208 | butyl 2,7,7-tnmethyl-4-naphthyl-5- oxo-1,4,6,7,8-pentahydroquinoline- 3-carboxylate | 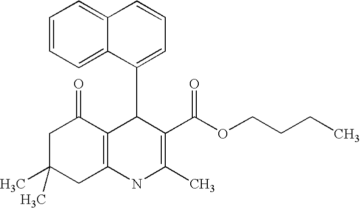 | 0 | 20.19103421 |
| ST013212 | phenylmethyl 4-[4- (dimethylamino)phenyl]-2-methyl- 5-oxo-1,4,6,7,8-pentahydroquinoline- 3-carboxylate | 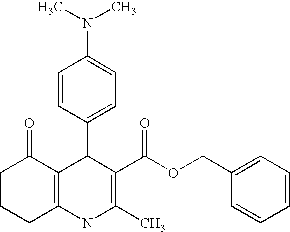 | 50.84142151 | 93.67934224 |
| ST013213 | ethyl 4-(2,4-dimethoxyphenyl)-2- methyl-5-oxo-1,4,6,7,8- pentahydroquinoline-3-carboxylate | 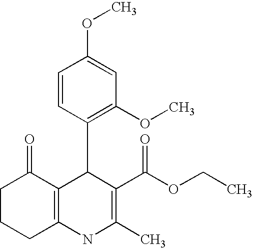 | 78.23840694 | 69.34764412 |
| ST013214 | phenylmethyl 2,7,7-trimethyl-4-(4- nitrophenyl)-5-oxo-1,4,6,7,8- pentahydroquinoline-3-carboxylate | 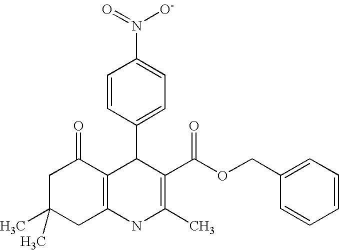 | 0 | 4.363166315 |
| ST013215 | phenylmethyl 2,7,7-trimethyl-5- oxo-4-phenyl-1,4,6,7,8- pentahydroquinoline-3-carboxylate | 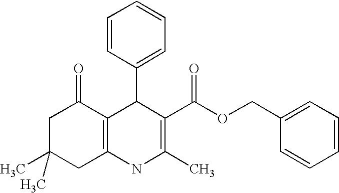 | 0 | 88.64072218 |
| ST013216 | ethyl 2-methyl-5-oxo-4-[2- (phenylmethoxy)phenyl]-1,4,6,7,8- pentahydroquinoline-3-carboxylate | 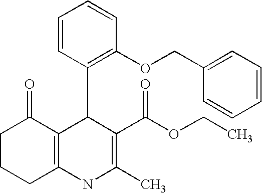 | 0 | 88.75568176 |
| ST013227 | cyclopentyl 4-(4-ethylphenyl)- 2,7,7-trimethyl-5-oxo-1,4,6,7,8- pentahydroquinoline-3-carboxylate | 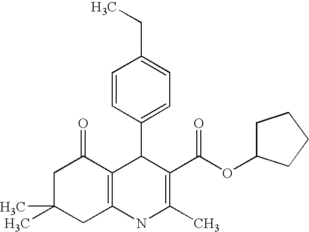 | 0 | 92.65318231 |
| ST013230 | phenylmethyl 4-(2,3- dichlorophenyl)-2,7,7-trimethyl-5- oxo-1,4,6,7,8-pentahydroquinoline- 3-carboxylate | 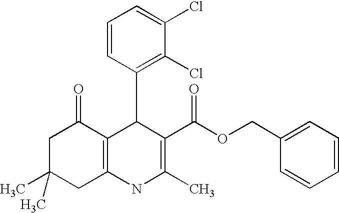 | 0 | 69.46790138 |
| ST013235 | cyclohexyl 4-(2-chlorophenyl)- 2,7,7-trimethyl-5-oxo-1,4,6,7,8- pentahydroquinoline-3-carboxylate | 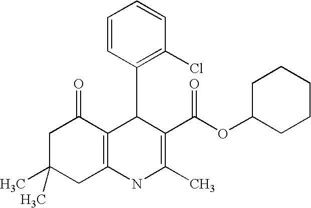 | 0 | 43.00070989 |
| ST013239 | butyl 4-(2,3-dichlorophenyl)-2,7,7- trimethyl-5-oxo-1,4,6,7,8- pentahydroquinoline-3-carboxylate | 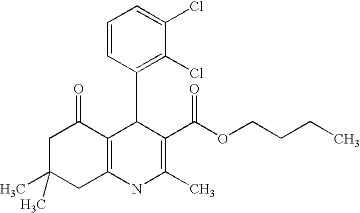 | 0 | 59.45264407 |
| ST013242 | phenylmethyl 4-(3-ethoxy-4- hydroxyphenyl)-2-methyl-5-oxo- 1,4,6,7,8-pentahydroquinoline-3- carboxylate | 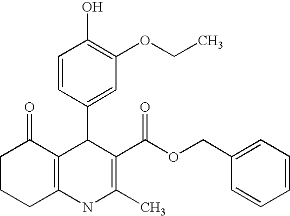 | 81.24629031 | 64.37418548 |
| ST013244 | propyl 4-(4-ethylphenyl)-2-methyl- 5-oxo-1,4,6,7,8- pentahydroquinoline-3-carboxylate | 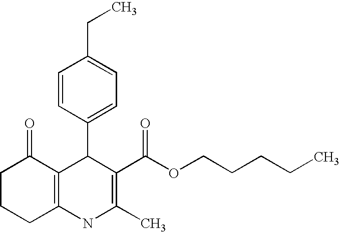 | 32.20558607 | 57.5873322 |
| ST013247 | propyl 2-methyl-4-(4- methylphenyl)-5-oxo-1,4,6,7,8- pentahydroquinoline-3-carboxylate | 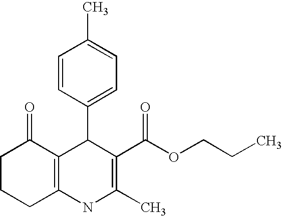 | 35.51944089 | 54.95756561 |
| ST013249 | phenylmethyl 4-(3-bromo-4- hydroxy-5-methoxyphenyl)-2- methyl-5-oxo-1,4,6,7,8- pentahydroquinoline-3-carboxylate | 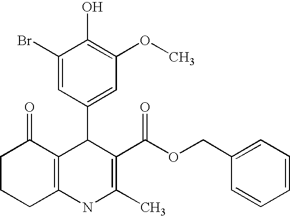 | 0 | 68.02004641 |
| ST013251 | 2-methoxyethyl 4-[4- (dimethylamino)phenyl]-2-methyl- 5-oxo-1,4,6,7,8-pentahydroquinoline- 3-carboxylate | 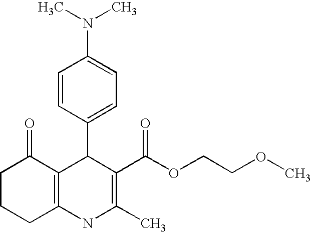 | 89.42392095 | 58.58382513 |
| ST013253 | 2-methoxyethyl 2-methyl-5-oxo-4- phenyl-1,4,6,7,8- pentahydroquinoline-3-carboxylate | 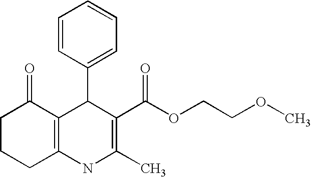 | 50.23449452 | 85.75401829 |
| ST013254 | 2-methoxyethyl 4-(4- hydroxyphenyl)-2-methyl-5-oxo- 1,4,6,7,8-pentahydroquinoline-3- carboxylate | 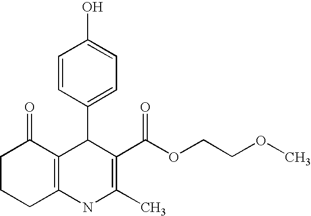 | 49.6225516 | 76.86398746 |
| ST013256 | 2-methoxyethyl 4-[4- (diethylamino)phenyl]-2-methyl-5- oxo-1,4,6,7,8-pentahydroquinoline- 3-carboxylate | 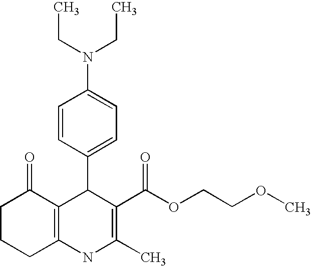 | 60.24795392 | 92.76708236 |
| ST013258 | cyclohexyl 2-methyl-4-naphthyl-5- oxo-1,4,6,7,8-pentahydroquinoline- 3-carboxylate | 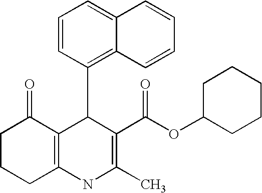 | 0 | 47.04760492 |
| ST013269 | cyclohexyl 4-(3,4-dichlorophenyl)- 2-methyl-5-oxo-1,4,6,7,8- pentahydroquinoline-3-carboxylate | 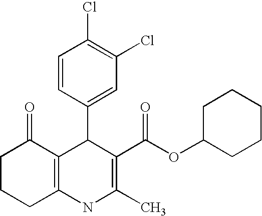 | 12.32832094 | 1.354161377 |
| ST013270 | cyclohexyl 4-(2,5- dimethoxyphenyl)-2-methyl-5-oxo- 1,4,6,7,8-pentahydroquinoline-3- carboxylate | 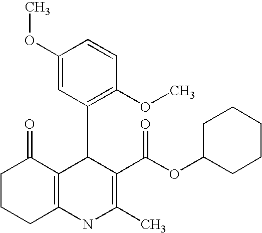 | 19.69762771 | 34.97746754 |
| ST013271 | cyclohexyl 2-methyl-5-oxo-4-[2- (phenylmethoxy)phenyl]-1,4,6,7,8- pentahydroquinoline-3-carboxylate | 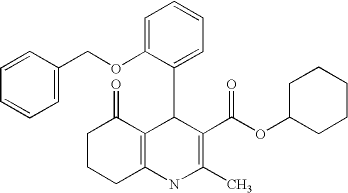 | 6.9200912 | 39.85192766 |
| ST013320 | cyclohexyl 4-(4-ethylphenyl)-2- methyl-5-oxo-1,4,6,7,8- pentahydroquinoline-3-carboxylate | 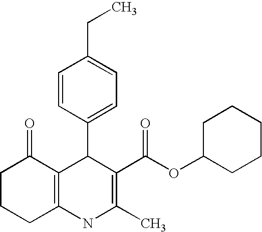 | 60.23152923 | 71.76951132 |
| ST013322 | propyl 2-methyl-4-[2- (methylethoxy)phenyl]-5-oxo- 1,4,6,7,8-pentahydroquinoline-3- carboxylate | 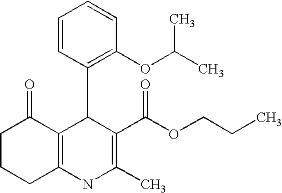 | 57.57423593 | 99.72356879 |
| ST013323 | methylethyl 4-(4-ethoxy-3- methoxyphenyl)-2-methyl-5-oxo- 1,4,6,7,8-pentahydroquinoline-3- carboxylate | 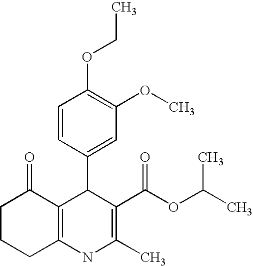 | 56.67309049 | 80.07468721 |
| ST013324 | butyl 4-(2-methoxyphenyl)-2- methyl-5-oxo-1,4,6,7,8- pentahydroquinoline-3-carboxylate | 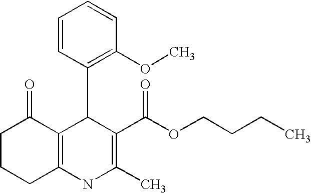 | 54.26551219 | 93.68192572 |
| ST013325 | butyl 2-methyl-5-oxo-4-(2- propoxyphenyl)-1,4,6,7,8- pentahydroquinoline-3-carboxylate | 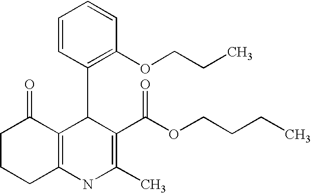 | 28.46208132 | 69.39399976 |
| ST013328 | cyclopentyl 2,7,7-trimethyl-5-oxo- 4-(2,3,4-trimethoxyphenyl)- 1,4,6,7,8-pentahydroquinoline-3- carboxylate | 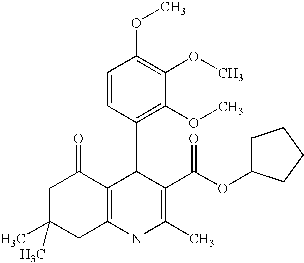 | 43.10840888 | 93.41265594 |
| ST013330 | butyl 4-(2-butoxyphenyl)-2-methyl- 5-oxo-1,4,6,7,8- pentahydroquinoline-3-carboxylate | 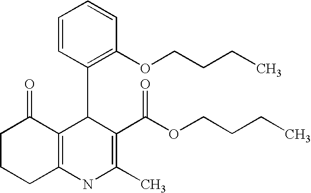 | 37.99196569 | 86.50382876 |
| ST013332 | methyl 7-(4-methoxyphenyl)-2- methyl-4-(4-methylphenyl)-5-oxo- 1,4,6,7,8-pentahydroquinoline-3- carboxylate | 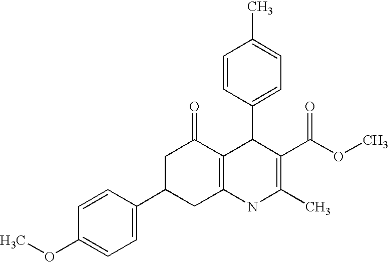 | 45.08034309 | 67.99192191 |
| ST013333 | methyl 7-(4-methoxyphenyl)-2- methyl-4-(3-nitrophenyl)-5-oxo- 1,4,6,7,8-pentahydroquinoline-3- carboxylate | 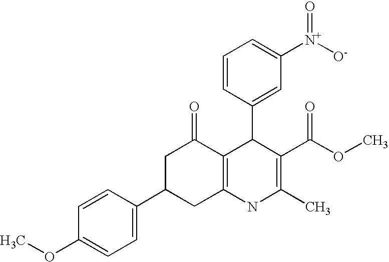 | 48.38228109 | 51.55865277 |
| ST013339 | cyclohexyl 4-(2-methoxyphenyl)-2- methyl-5-oxo-1,4,6,7,8- pentahydroquinoline-3-carboxylate | 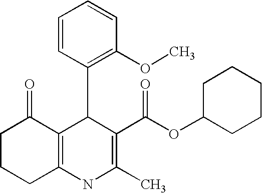 | 43.6404104 | 6.873410651 |
| ST013341 | 2-ethoxyethyl 4-(3,4- dimethoxyphenyl)-2-methyl-5-oxo- 1,4,6,7,8-pentahydroquinoline-3- carboxylate | 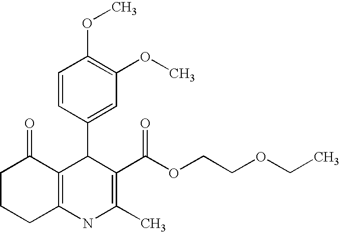 | 73.01313718 | 73.85700316 |
| ST013342 | 2-ethoxyethyl 4-(3- hydroxyphenyl)-2-methyl-5-oxo- 1,4,6,7,8-pentahydroquinoline-3- carboxylate | 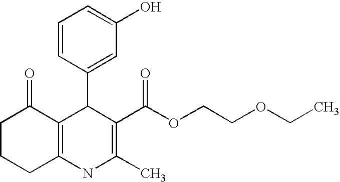 | 69.74512784 | 55.47322081 |
| ST013343 | 2-ethoxyethyl 4-(4-hydroxy-3- methoxyphenyl)-2,7,7-trimethyl-5- oxo-1,4,6,7,8-pentahydroquinoline- 3-carboxylate | 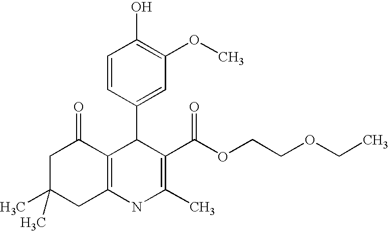 | 61.10146029 | 82.95488429 |
| ST013344 | 2-ethoxyethyl 4-(4-ethylphenyl)-2- methyl-5-oxo-1,4,6,7,8- pentahydroquinoline-3-carboxylate | 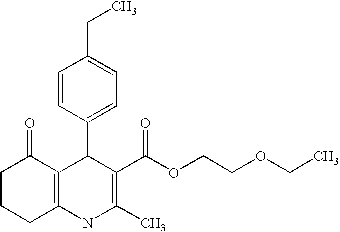 | 54.38086966 | 86.63976573 |
| ST013345 | 2-ethoxyethyl 2-methyl-5-oxo-4- (3,4,5-trimethoxyphenyl)-1,4,6,7,8- pentahydroquinoline-3-carboxylate | 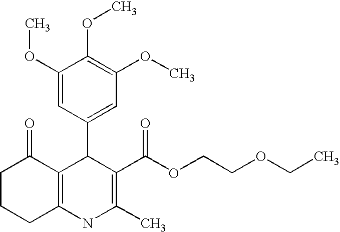 | 58.35595245 | 75.38303887 |
| ST013346 | pentyl 2-methyl-5-oxo-4-(2,3,4- trimethoxyphenyl)-1,4,6,7,8- pentahydroquinoline-3-carboxylate | 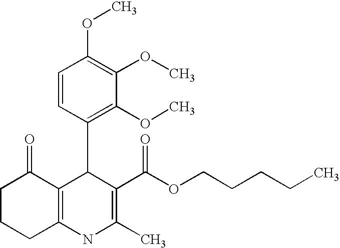 | 16.27626079 | 77.73355052 |
| ST013350 | cyclohexyl 4-(3,4-dichlorophenyl)- 2-methyl-5-oxo-7-phenyl-1,4,6,7,8- pentahydroquinoline-3-carboxylate | 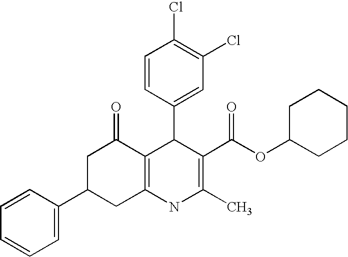 | 25.07464307 | 95.56681419 |
| ST013352 | cyclohexyl 4-(4-chlorophenyl)-2- methyl-5-oxo-7-phenyl-1,4,6,7,8- pentahydroquinoline-3-carboxylate | 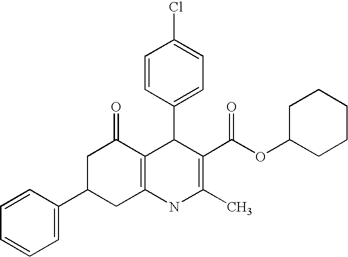 | 22.38613539 | 84.04915085 |
| ST013356 | 2-methoxyethyl 2,7,7-trimethyl-5- oxo-4-(3,4,5-trimethoxyphenyl)- 1,4,6,7,8-pentahydroquinoline-3- carboxylate | 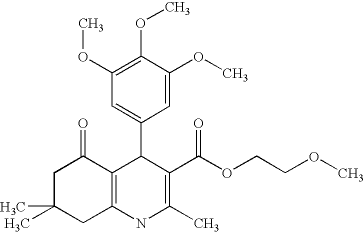 | 62.71375061 | 82.54446923 |
| ST013357 | 2-methoxyethyl 4-(3-bromo-4- hydroxy-5-methoxyphenyl)-2- methyl-5-oxo-1,4,6,7,8- pentahydroquinoline-3-carboxylate | 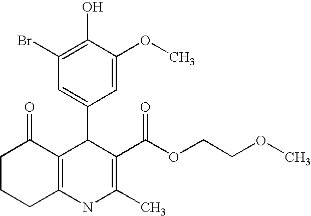 | 62.39346398 | 95.16316994 |
| ST013365 | 2-methoxyethyl 4-(2- methoxyphenyl)-2-methyl-5-oxo- 1,4,6,7,8-pentahydroquinoline-3- carboxylate | 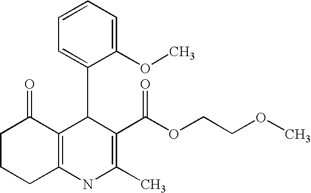 | 84.53802725 | 56.04978106 |
| ST013387 | cyclohexyl 4-(4-hydroxy-3- methoxyphenyl)-2-methyl-5-oxo-7- phenyl-1,4,6,7,8- pentahydroquinoline- 3-carboxylate | 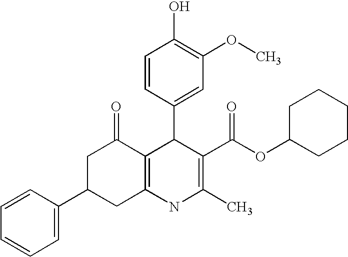 | 39.97475707 | 26.3587512 |
| ST013389 | cyclohexyl 4-(4-ethylphenyl)-2- methyl-5-oxo-7-phenyl-1,4,6,7,8- pentahydroquinoline-3-carboxylate | 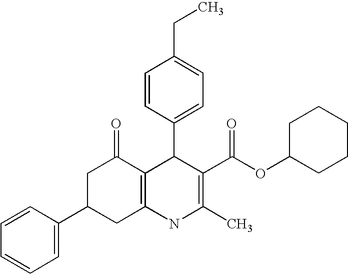 | 37.41382118 | 94.35588142 |
| ST013390 | phenylmethyl 4-(4-ethylphenyl)-2- methyl-5-oxo-7-phenyl-1,4,6,7,8- pentahydroquinoline-3-carboxylate | 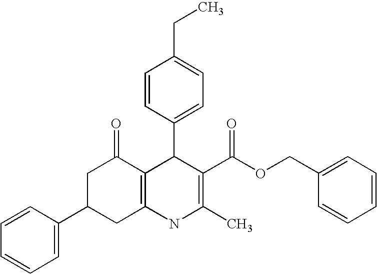 | 23.36870962 | 98.25117871 |
| ST013410 | 4-(4-chlorophenyl)-2,7,7-trimethyl- 5-oxo-1,4,6,7,8- pentahydroquinoline-3-carbonitrile | 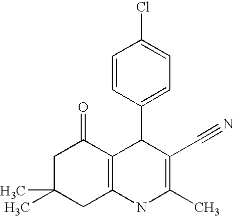 | 21.9857771 | 26.03843995 |
| ST013428 | butyl 4-(3-chlorophenyl)-2-methyl- 5-oxo-1,4,6,7,8- pentahydroquinoline-3-carboxylate | 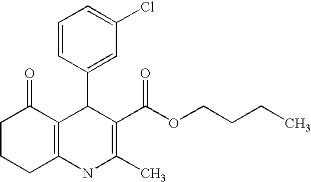 | 25.07464307 | 40.90661104 |
| ST013429 | cyclohexyl 4-(3-bromo-4- methoxyphenyl)-2-methyl-5-oxo- 1,4,6,7,8-pentahydroquinoline-3- carboxylate | 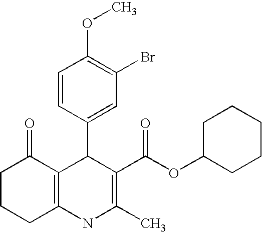 | 31.93773411 | 45.38628104 |
| ST013435 | 4-{3-[(2-ethoxyethyl)oxycarbonyl]- 2-methyl-5-oxo-4-1,4,6,7,8- pentahydroquinolyl}phenyl acetate | 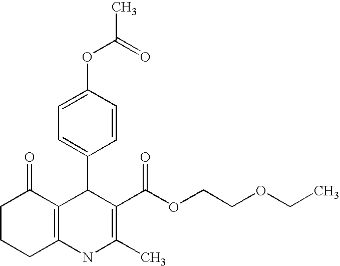 | 64.72232778 | 70.09399703 |
| ST013438 | cyclohexyl 2-methyl-4-(4- methylphenyl)-5-oxo-7-phenyl- 1,4,6,7,8-pentahydroquinoline-3- carboxylate | 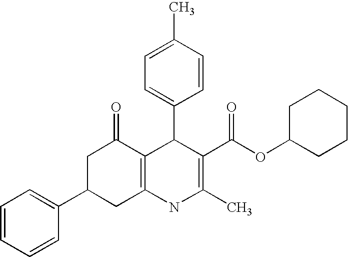 | 31.45594702 | 93.54703042 |
| ST013439 | methylethyl 4-(2,3-dichlorophenyl)- 2-methyl-5-oxo-7-phenyl-1,4,6,7,8- pentahydroquinoline-3-carboxylate | 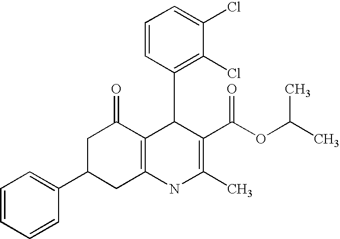 | 19.69762771 | 80.34968613 |
| ST014138 | 2-amino-7,7-dimethyl-5-oxo-1- benzylspiro[1,4,6,7,8- pentahydroquinoline-4,4′-2′ H- 3′,4,5′,6′-tetrahydropyran]-3- carbonitrile | 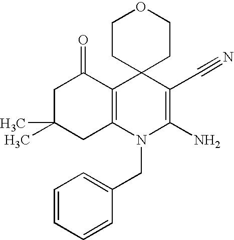 | 64.09261169 | 65.31693236 |
| ST014234 | [4-(2-amino-3-cyano-7,7-dimethyl- 5-oxo-1-phenyl-6,8-dihydro-4H- quinolin-4-yl)phenoxy]sodium | 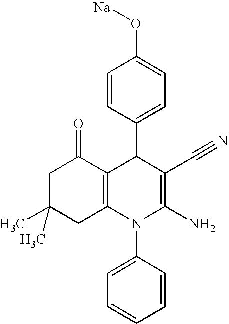 | 83.72509636 | 63.62006398 |
| ST014875 | methylethyl 4-[4- (diethylamino)phenyl]-2-methyl-5- oxo-7-phenyl-1,4,6,7,8- pentahydroquinoline- 3-carboxylate | 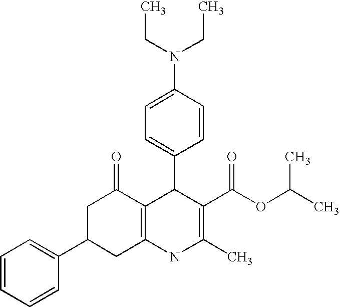 | 54.1884249 | 95.73807992 |
| ST014881 | phenylmethyl 2,7,7-trimethyl-5- oxo-4-[2-(trifluoromethyl)phenyl]- 1,4,6,7,8-pentahydroquinoline-3- carboxylate | 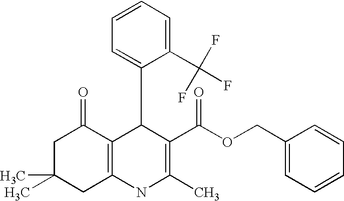 | 0 | 49.88766565 |
| ST014882 | cyclohexyl 2-methyl-5-oxo-4-[2- (trifluoromethyl)phenyl]-1,4,6,7,8- pentahydroquinoline-3-carboxylate | 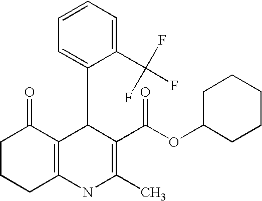 | 39.46178615 | 91.81548227 |
| ST014884 | 2-phenylethyl 4-(2,4- dichlorophenyl)-2-methyl-5-oxo- 1,4,6,7,8-pentahydroquinoline-3- carboxylate | 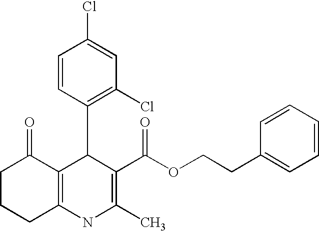 | 61.97300589 | 52.78225692 |
| ST014885 | 2-phenylethyl 4-(6-bromo(2H- benzo[d]1,3-dioxolen-5-yl))-2,7,7- trimethyl-5-oxo-1,4,6,7,8- pentahydroquinoline-3-carboxylate | 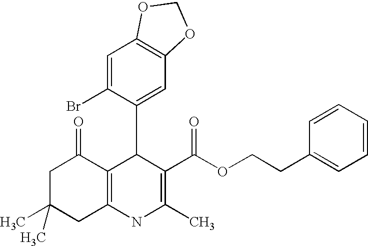 | 39.46178615 | 87.49242738 |
| ST014886 | 2-phenoxyethyl 4-(4-ethylphenyl)- 2,7,7-trimethyl-5-oxo-1,4,6,7,8- pentahydroquinoline-3-carboxylate | 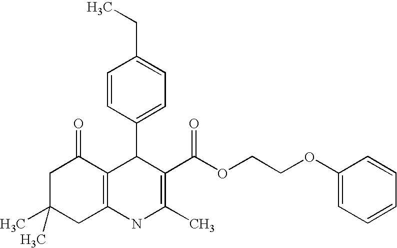 | 21.59695691 | 65.06737638 |
| ST014890 | 2-phenoxyethyl 4-(4- bromophenyl)-2-methyl-5-oxo- 1,4,6,7,8-pentahydroquinoline-3- carboxylate | 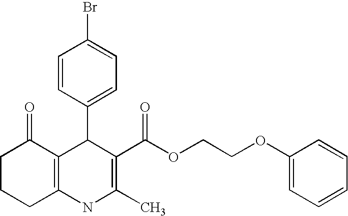 | 13.21343366 | 67.45959598 |
| ST014891 | 2-phenoxyethyl 4-(4-fluorophenyl)- 2-methyl-5-oxo-1,4,6,7,8- pentahydroquinoline-3-carboxylate | 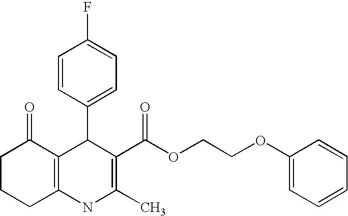 | 51.76095697 39.34284581 | |
| ST014899 | 2-phenoxyethyl 2-methyl-5-oxo-4- [4-(phenylmethoxy)phenyl]- 1,4,6,7,8-pentahydroquinoline-3- carboxylate | 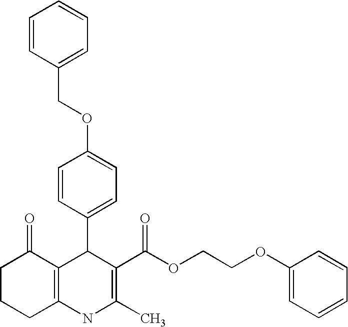 | 55.6115384 | 65.46783361 |
| ST014907 | 2-phenylethyl 4-(2-butoxyphenyl)- 2,7,7-trimethyl-5-oxo-1,4,6,7,8- pentahydroquinoline-3-carboxylate | 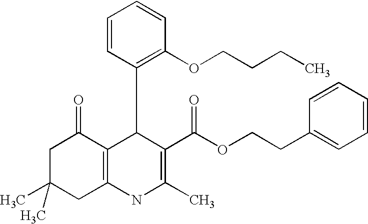 | 34.56680959 | 83.50391768 |
| ST014919 | 2-phenoxyethyl 2-methyl-4-[2- (methylethoxy)phenyl]-5-oxo- 1,4,6,7,8-pentahydroquinoline-3- carboxylate | 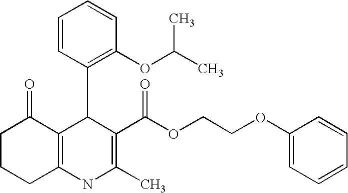 | 63.42614992 | 85.12900968 |
| ST014923 | 2-phenylethyl 2,7,7-trimethyl-4-(4- nitrophenyl)-5-oxo-1,4,6,7,8- pentahydroquinoline-3-carboxylate | 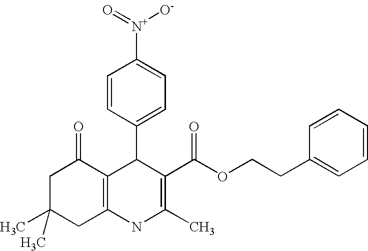 | 0 | 28.35824306 |
| ST014957 | phenylmethyl 2-methyl-5-oxo-4-(2- propoxyphenyl)-1,4,6,7,8- pentahydroquinoline-3-carboxylate | 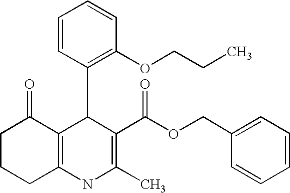 | 0 | 62.7831639 |
| ST014958 | propyl 4-(2,3-dichlorophenyl)-2- methyl-5-oxo-1,4,6,7,8- pentahydroquinoline-3-carboxylate | 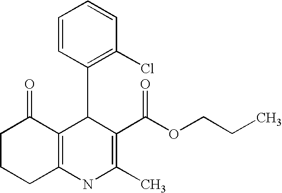 | 18.22853234 | 61.15530249 |
| ST014959 | propyl 4-(3-hydroxyphenyl)-2- methyl-5-oxo-1,4,6,7,8- pentahydroquinoline-3-carboxylate | 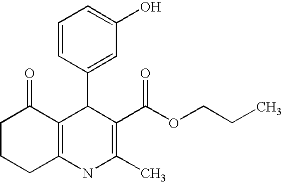 | 64.0017351 | 70.21959374 |
| ST014969 | prop-2-enyl 2-methyl-5-oxo-4- (2,3,4-trimethoxyphenyl)-1,4,6,7,8- pentahydroquinoline-3-carboxylate | 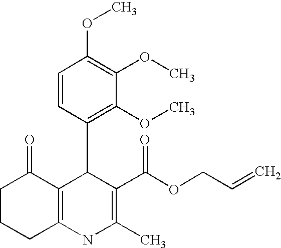 | 39.6569846 | 83.25356268 |
| ST015031 | phenylmethyl 4-(4-ethylphenyl)-2- methyl-5-oxo-1,4,6,7,8- pentahydroquinoline-3-carboxylate | 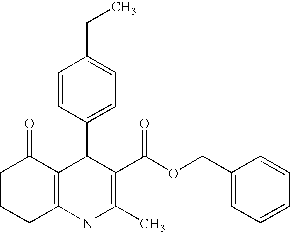 | 33.05193614 | 71.26310885 |
| ST015043 | methylethyl 4-(3-ethoxy-4- hydroxyphenyl)-2-methyl-5-oxo- 1,4,6,7,8-pentahydroquinoline-3- carboxylate | 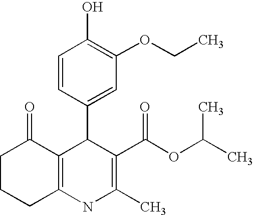 | 95.93419977 | 68.77783694 |
| ST015048 | 2-phenylethyl 2,7,7-trimethyl-5- oxo-4-(2-propoxyphenyl)-1,4,6,7,8- pentahydroquinoline-3-carboxylate | 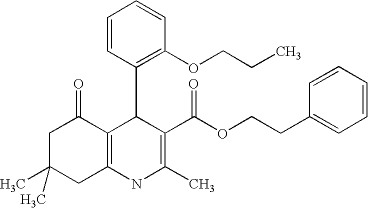 | 37.87517309 | 88.7314631 |
| ST015049 | 2-phenylethyl 2,7,7-trimethyl-4-[2- (methylethoxy)phenyl]-5-oxo- 1,4,6,7,8-pentahydroquinoline-3- carboxylate | 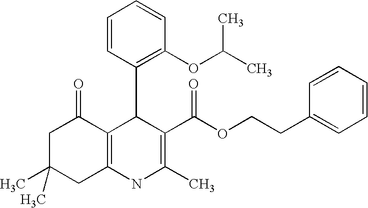 | 0 | 62.37716785 |
| ST015054 | 2-phenylethyl 2,7,7-trimethyl-5- oxo-4-phenyl-1,4,6,7,8- pentahydroquinoline-3-carboxylate | 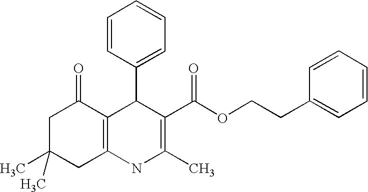 | 29.87871002 | 44.20316696 |
| ST015055 | 2-phenoxyethyl 2,7,7-trimethyl-5- oxo-4-(3-phenoxyphenyl)-1,4,6,7,8- pentahydroquinoline-3-carboxylate | 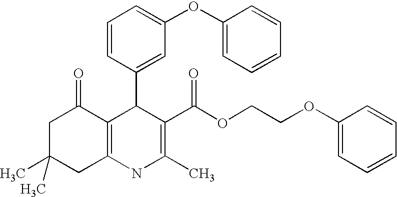 | 13.61050401 | 78.20602915 |
| ST015060 | 2-phenylethyl 2-methyl-5-oxo-4-(2- propoxyphenyl)-1,4,6,7,8- pentahydroquinoline-3-carboxylate | 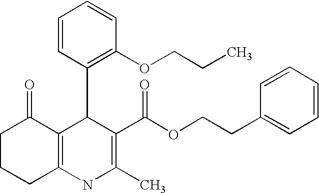 | 0 | 0 |
| ST015061 | 2-phenylethyl 4-(3-bromo-5- ethoxy-4-hydroxyphenyl)-2- methyl-5-oxo-1,4,6,7,8- pentahydroquinoline-3-carboxylate | 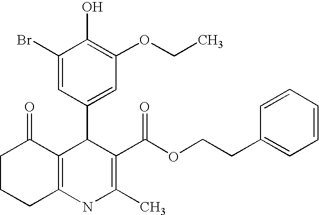 | 40.81482841 | 33.53150797 |
| ST015062 | 2-phenylethyl 4-(2,3- dichlorophenyl)-2-methyl-5-oxo- 1,4,6,7,8-pentahydroquinoline-3- carboxylate | 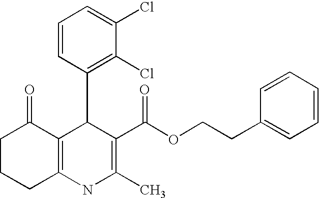 | 30.57608569 | 20.81712938 |
| ST015067 | 2-phenoxyethyl 4-(2,4- dichlorophenyl)-2,7,7-trimethyl-5- oxo-1,4,6,7,8- pentahydroquinoline-3-carboxylate | 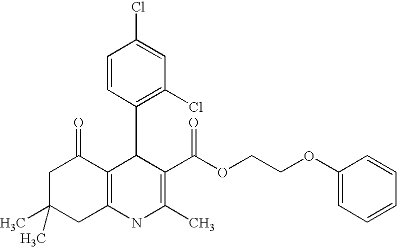 | 4.89497656 | 43.89631592 |
| ST015068 | ethyl 4-(2,3-dichlorophenyl)-7-(4- chlorophenyl)-2-methyl-5-oxo- 1,4,6,7,8-pentahydroquinoline-3- carboxylate | 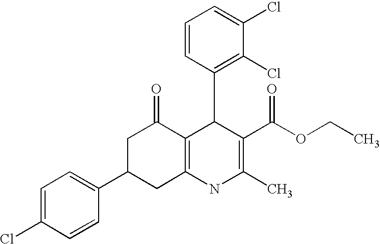 | 46.32042577 | 83.75427269 |
| ST015070 | methyl 7-(2-chlorophenyl)-2- methyl-4-(3-nitrophenyl)-5-oxo- 1,4,6,7,8-pentahydroquinoline-3- carboxylate | 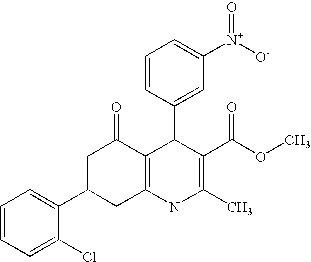 | 65.42818532 | 96.95883751 |
| ST015071 | 2-phenoxyethyl 4-[3-methoxy-4- (phenylmethoxy)phenyl]-2,7,7- trimethyl-5-oxo-1,4,6,7,8- pentahydroquinoline-3-carboxylate | 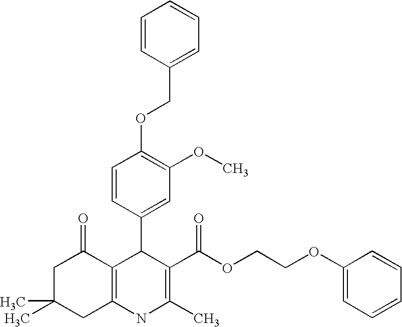 | 60.82552896 | 88.32224232 |
| ST015072 | 2-phenoxyethyl 2-methyl-4-(4- methylthiophenyl)-5-oxo-1,4,6,7,8- pentahydroquinoline-3-carboxylate | 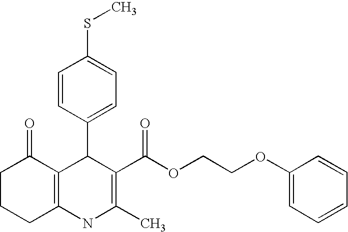 | 19.33402706 | 59.38239758 |
| ST015075 | 2-phenoxyethyl 4-[4- (diethylamino)phenyl]-2,7,7- trimethyl-5-oxo-1,4,6,7,8- pentahydroquinoline- 3-carboxylate | 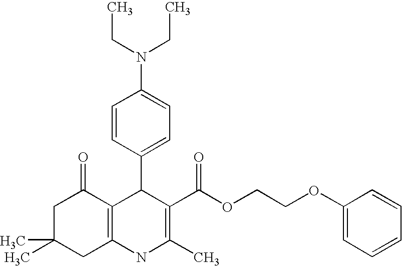 | 51.66666667 | 42.20603587 |
| ST015076 | 2-phenylethyl 2-methyl-4-(4- methylphenyl)-5-oxo-1,4,6,7,8- pentahydroquinoline-3-carboxylate | 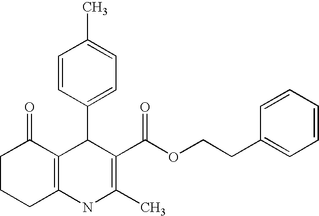 | 58.90912244 | 98.56122264 |
| ST015084 | 2-phenylethyl 4-(3-chlorophenyl)- 2,7,7-trimethyl-5-oxo-1,4,6,7,8- pentahydroquinoline-3-carboxylate | 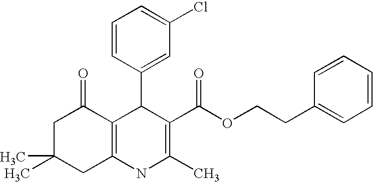 | 59.46063129 | 80.23086604 |
| ST015085 | 2-phenylethyl 4-(2-chlorophenyl)- 2,7,7-trimethyl-5-oxo-1,4,6,7,8- pentahydroquinoline-3-carboxylate | 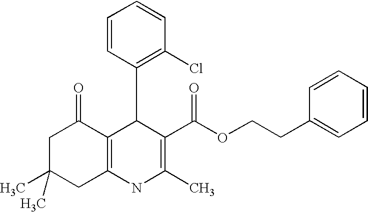 | 69.89594173 | 72.88944673 |
| ST015087 | 2-phenylethyl 4-(4-ethoxyphenyl)- 2,7,7-trimethyl-5-oxo-1,4,6,7,8- pentahydroquinoline-3-carboxylate | 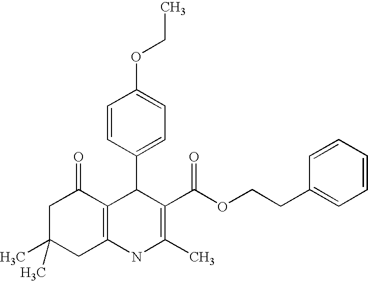 | 35.27055151 | 43.7042733 |
| ST015090 | 2-phenylethyl 4-(2,4- dimethoxyphenyl)-2,7,7-trimethyl- 5-oxo-1,4,6,7,8- pentahydroquinoline- 3-carboxylate | 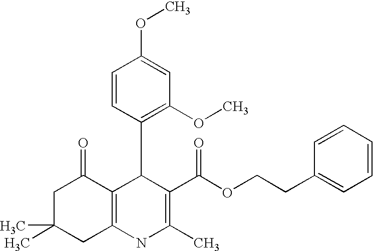 | 15.37807839 | 34.94397117 |
| ST015091 | 2-phenoxyethyl 4-(3-chlorophenyl)- 2-methyl-5-oxo-1,4,6,7,8- pentahydroquinoline-3-carboxylate | 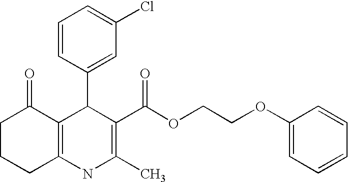 | 62.44536941 | 75.84121477 |
| ST015093 | 2-phenoxyethyl 4-(4-chlorophenyl)- 2,7,7-trimethyl-5-oxo-1,4,6,7,8- pentahydroquinoline-3-carboxylate | 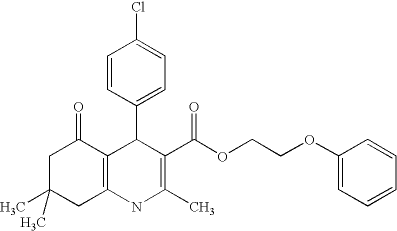 | 0 | 0 |
| ST015094 | 2-phenylethyl 4-(4-hydroxy-3- methoxyphenyl)-2,7,7-trimethyl-5- oxo-1,4,6,7,8- pentahydroquinoline- 3-carboxylate | 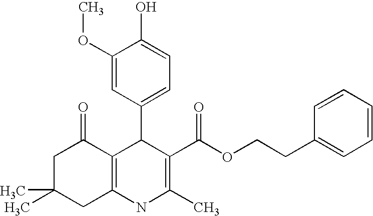 | 56.26257371 | 59.8054359 |
| ST015095 | 2-phenoxyethyl 2,7,7-trimethyl-4- (4-methylphenyl)-5-oxo-1,4,6,7,8- pentahydroquinoline-3-carboxylate | 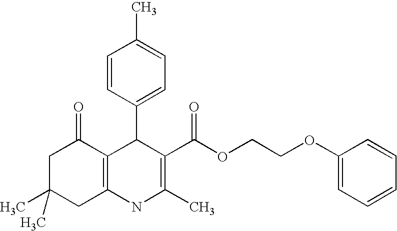 | 59.7346514 | 0 |
| ST015096 | 2-phenoxyethyl 4-(2-chlorophenyl)- 2,7,7-trimethyl-5-oxo-1,4,6,7,8- pentahydroquinoline-3-carboxylate | 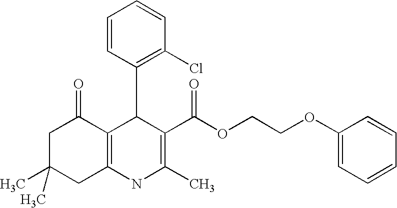 | 28.05931322 | 79.96653694 |
| ST015098 | 2-phenylethyl 4-(3,4- dichlorophenyl)-2-methyl-5-oxo- 1,4,6,7,8-pentahydroquinoline-3- carboxylate | 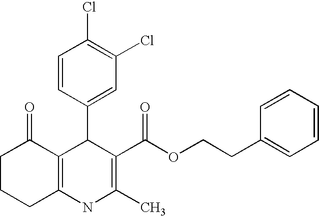 | 40.85848075 | 0 |
| ST015099 | 2-phenylethyl 2,7,7-trimethyl-4-(4- methylphenyl)-5-oxo-1,4,6,7,8- pentahydroquinoline-3-carboxylate | 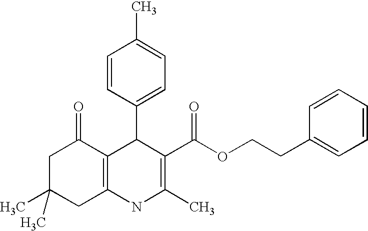 | 14.8491155 | 33.8235064 |
| ST015103 | 2-phenoxyethyl 4-(2H-benzo[3,4- d]1,3-dioxolan-5-yl)-2,7,7- trimethyl-5-oxo-1,4,6,7,8- pentahydroquinoline-3-carboxylate | 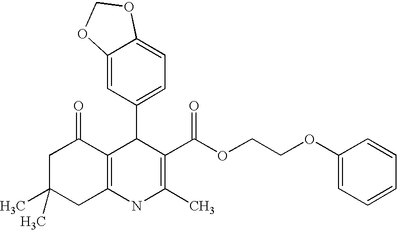 | 35.27055151 | 73.49466536 |
| ST015105 | 2-phenoxyethyl 2,7,7-trimethyl-5- oxo-4-(2-propoxyphenyl)-1,4,6,7,8- pentahydroquinoline-3-carboxylate | 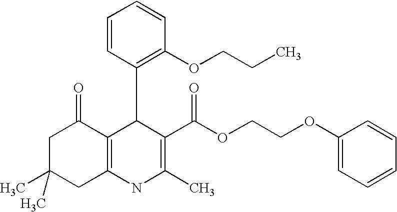 | 52.9778009 | 99.75042416 |
| ST015106 | 2-phenylethyl 4-(4-chlorophenyl)- 2,7,7-trimethyl-5-oxo-1,4,6,7,8- pentahydroquinoline-3-carboxylate | 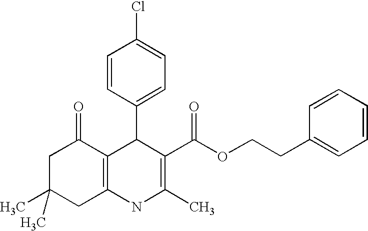 | 13.2049948 | 0 |
| ST015108 | 2-phenoxyethyl 4-(2- methoxyphenyl)-2,7,7-trimethyl-5- oxo-1,4,6,7,8- pentahydroquinoline- 3-carboxylate | 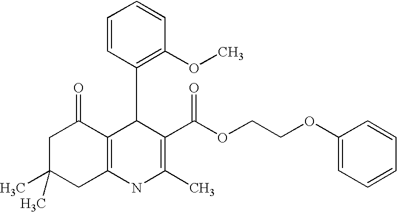 | 32.95178633 | 92.11729282 |
| ST015110 | 2-phenylethyl 4-(2- methoxyphenyl)-2-methyl-5-oxo- 1,4,6,7,8-pentahydroquinoline-3- carboxylate | 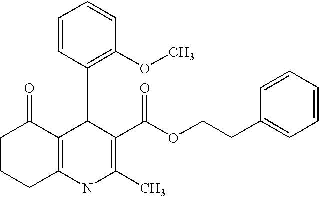 | 42.79916753 | 90.13454519 |
| ST015159 | cyclohexyl 4-(3-chlorophenyl)-2- methyl-5-oxo-1,4,6,7,8- pentahydroquinoline-3-carboxylate | 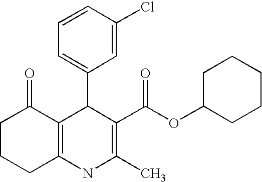 | 2.977800902 | 0 |
| ST015174 | pentyl 4-(2-ethoxyphenyl)-2- methyl-5-oxo-1,4,6,7,8- pentahydroquinoline-3-carboxylate | 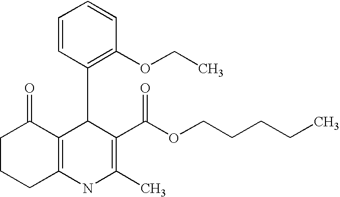 | 17.16441207 | 0 |
| ST015176 | methyl 7-(2-chlorophenyl)-2- methyl-4-(4-nitrophenyl)-5-oxo- 1,4,6,7,8-pentahydroquinoline-3- carboxylate | 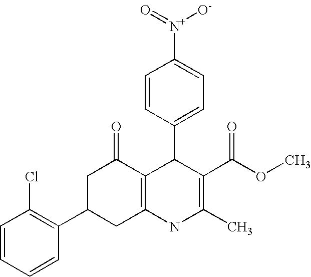 | 17.41068332 | 76.41122678 |
| ST015182 | methyl 4-[4-(diethylamino)phenyl]- 7-(2-chlorophenyl)-2-methyl-5- oxo-1,4,6,7,8- pentahydroquinoline-3-carboxylate | 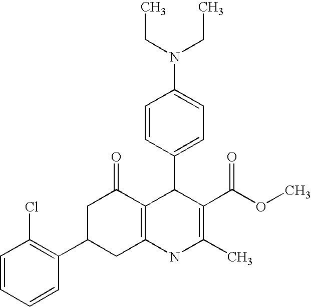 | 39.86992716 | 61.44841168 |
| ST015214 | methyl 7-(4-chlorophenyl)-2- methyl-4-(4-methylphenyl)-5-oxo- 1,4,6,7,8-pentahydroquinoline-3- carboxylate | 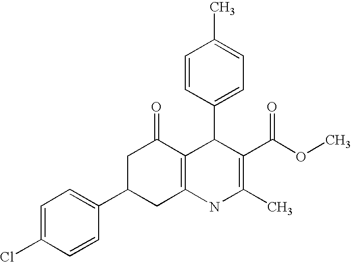 | 60.69025321 | 66.69363978 |
| ST015215 | methyl 4-(3,4-dichlorophenyl)-7-(4- chlorophenyl)-2-methyl-5-oxo- 1,4,6,7,8-pentahydroquinoline-3- carboxylate | 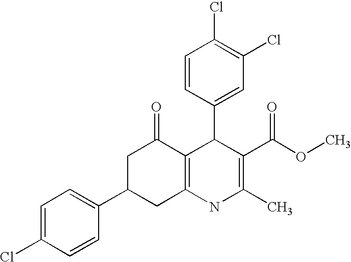 | 23.14429414 | 39.7605726 |
| ST015216 | methyl 7-(4-chlorophenyl)-4-(2- fluorophenyl)-2-methyl-5-oxo- 1,4,6,7,8-pentahydroquinoline-3- carboxylate | 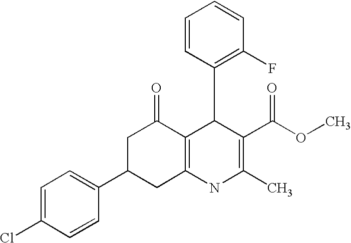 | 68.3593479 | 93.19472728 |
| ST015218 | methyl 7-(4-chlorophenyl)-2- methyl-5-oxo-4-phenyl-1,4,6,7,8- pentahydroquinoline-3-carboxylate | 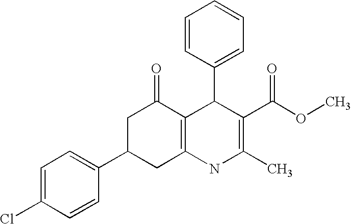 | 59.32362123 | 0 |
| ST015219 | 2-methoxyethyl 4-(3- methoxyphenyl)-2-methyl-5-oxo- 1,4,6,7,8-pentahydroquinoline-3- carboxylate | 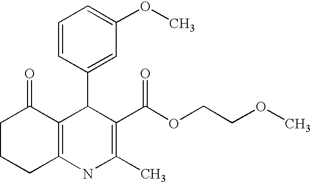 | 83.70620881 | 45.79375848 |
| ST015234 | 2-phenylethyl 4-(2-bromophenyl)- 2-methyl-5-oxo-7-phenyl-1,4,6,7,8- pentahydroquinoline-3-carboxylate | 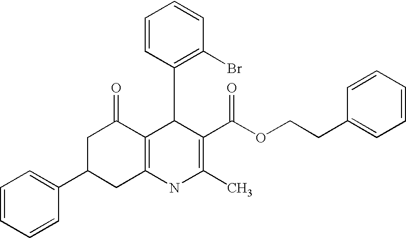 | 51.81408255 | 54.83794894 |
| ST015254 | ethyl 4-(2,5-dimethoxyphenyl)-2- methyl-5-oxo-7-phenyl-1,4,6,7,8- pentahydroquinoline-3-carboxylate | 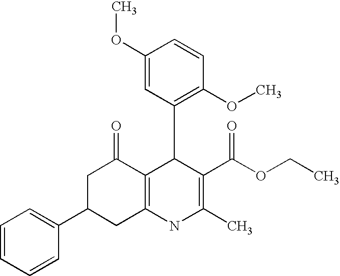 | 46.87304891 | 33.8235064 |
| ST015255 | 2-phenylethyl 2-methyl-4-(4- nitrophenyl)-5-oxo-7-phenyl- 1,4,6,7,8-pentahydroquinoline-3- carboxylate | 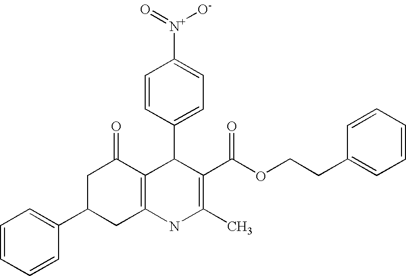 | 40.52896289 | 74.68219037 |
| ST015256 | ethyl 4-(2-fluorophenyl)-7-(4- methoxyphenyl)-2-methyl-5-oxo- 1,4,6,7,8-pentahydroquinoline-3- carboxylate | 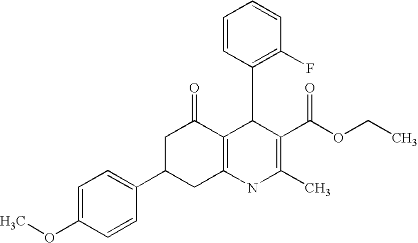 | 69.7693375 | 75.84121477 |
| ST016953 | methyl 7-(4-chlorophenyl)-2- methyl-5-oxo-4-[4- (phenylmethoxy)phenyl]-1,4,6,7,8- pentahydroquinoline-3-carboxylate | 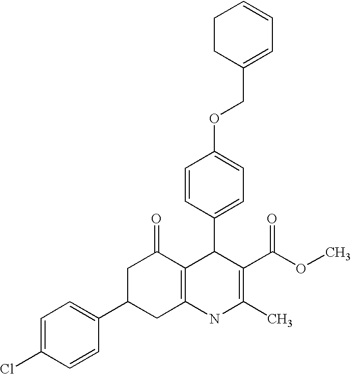 | 43.91432536 | 69.73817449 |
| ST016959 | (4-methoxyphenyl)methyl 4-(3- fluorophenyl)-2-methyl-5-oxo- 1,4,6,7,8-pentahydroquinoline-3- carboxylate | 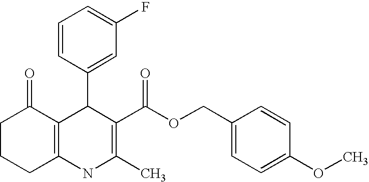 | 28.83801596 | 57.18729253 |
| ST016960 | 2-methoxyethyl 4-(4- bromophenyl)-7-(4-chlorophenyl)- 2-methyl-5-oxo-1,4,6,7,8- pentahydroquinoline- 3-carboxylate | 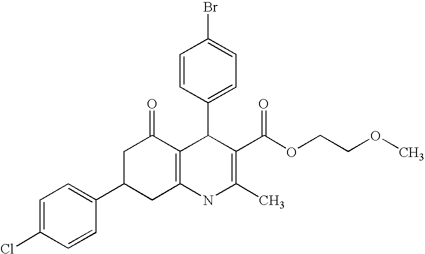 | 51.52098508 | 76.41122678 |
| ST016961 | methylethyl 7-(4-methoxyphenyl)- 2-methyl-5-oxo-4-[4- (phenylmethoxy)phenyl]-1,4,6,7,8- pentahydroquinoline-3-carboxylate | 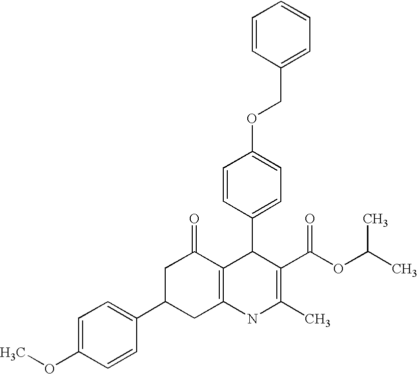 | 41.83489421 | 89.46114865 |
| ST016962 | methyl 4-(2-bromophenyl)-7-(4- methoxyphenyl)-2-methyl-5-oxo- 1,4,6,7,8-pentahydroquinoline-3- carboxylate | 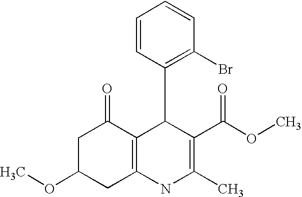 | 40.36593826 | 28.45868754 |
| ST016964 | (4-methoxyphenyl)methyl 4-(3- ethoxy-4-hydroxyphenyl)-2,7,7- trimethyl-5-oxo-1,4,6,7,8- pentahydroquinoline-3-carboxylate | 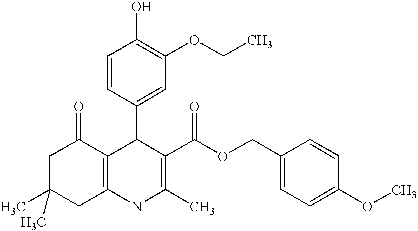 | 36.42611897 | 40.04974668 |
| ST016969 | 2-methylpropyl 4-(3-chlorophenyl)- 2-methyl-5-oxo-7-phenyl-1,4,6,7,8- pentahydroquinoline-3-carboxylate | 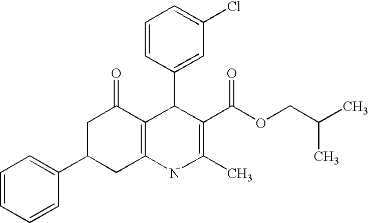 | 30.25476324 | 60.19647081 |
| ST016970 | 2-methylpropyl 4-(4-ethylphenyl)- 2-methyl-5-oxo-7-phenyl-1,4,6,7,8- pentahydroquinoline-3-carboxylate | 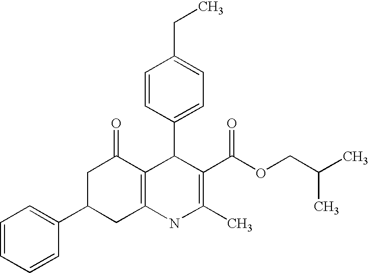 | 27.67223813 | 27.78307022 |
| ST016971 | 2-methoxyethyl 7-(4- chlorophenyl)-2-methyl-4-(4- nitrophenyl)-5-oxo-1,4,6,7,8- pentahydroquinoline- 3-carboxylate | 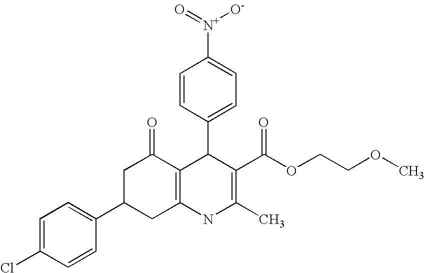 | 27.27539181 | 86.20182491 |
| ST016974 | methyl 4-(4-ethoxyphenyl)-7-(4- methoxyphenyl)-2-methyl-5-oxo- 1,4,6,7,8-pentahydroquinoline-3- carboxylate | 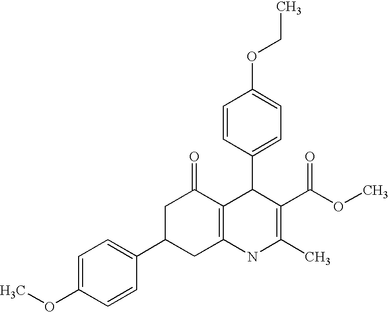 | 34.63449508 | 57.35636803 |
| ST016978 | 2-methylpropyl 4-(2H-benzo[3,4- d]1,3-dioxolen-5-yl)-2-methyl-5- oxo-7-phenyl-1,4,6,7,8- pentahydroquinoline-3-carboxylate | 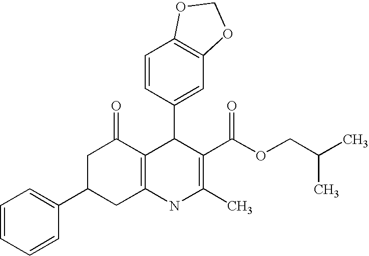 | 45.93600834 | 99.50423316 |
| ST016979 | (4-methoxyphenyl)methyl 4-(3- methoxy-4-propoxyphenyl)-2,7,7- trimethyl-5-oxo-1,4,6,7,8- pentahydroquinoline-3-carboxylate | 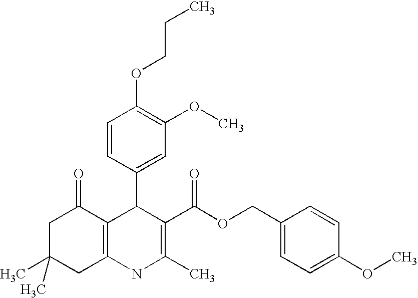 | 53.71335879 | 90.10594628 |
| ST016983 | (4-methoxyphenyl)methyl 4-(4- ethylphenyl)-2-methyl-5-oxo- 1,4,6,7,8-pentahydroquinoline-3- carboxylate | 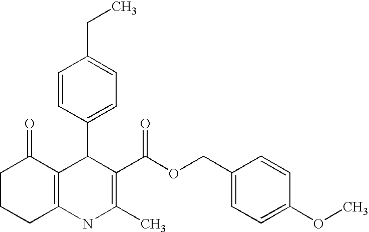 | 51.56489887 | 72.60730112 |
| ST016984 | 2-methylpropyl 2-methyl-5-oxo-7- phenyl-4-(2-propoxyphenyl)- 1,4,6,7,8-pentahydroquinoline-3- carboxylate | 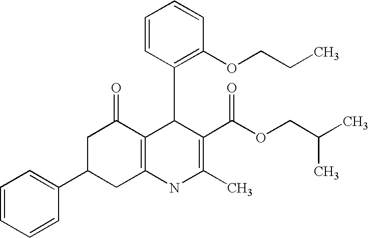 | 25.36373702 | 56.15200343 |
| ST016990 | methyl 7-(4-methoxyphenyl)-2- methyl-5-oxo-4-(4-propoxyphenyl)- 1,4,6,7,8-pentahydroquinoline-3- carboxylate | 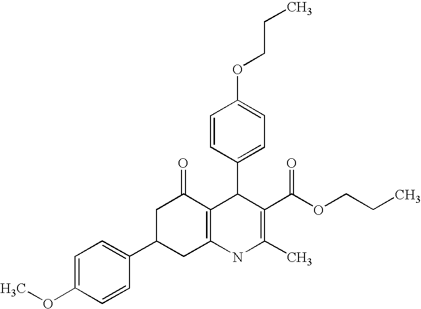 | 28.30077198 | 22.04955492 |
| ST016999 | methyl 4-(2,3-dimethoxyphenyl)-7- (4-methoxyphenyl)-2-methyl-5- oxo-1,4,6,7,8- pentahydroquinoline- 3-carboxylate | 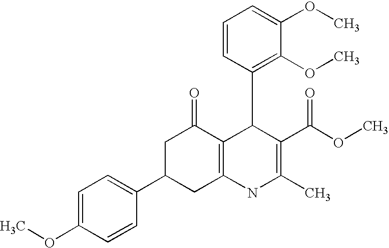 | 67.25265727 | 82.83569657 |
| ST017011 | methylethyl 4-(4-ethoxyphenyl)-7- (4-methoxyphenyl)-2-methyl-5- oxo-1,4,6,7,8- pentahydroquinoline- 3-carboxylate | 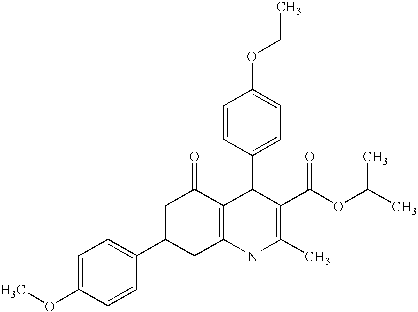 | 45.72432595 | 74.04122392 |
| ST017025 | (4-methoxyphenyl)methyl 4-(4- hydroxy-3-methoxyphenyl)-2,7,7- trimethyl-5-oxo-1,4,6,7,8- pentahydroquinoline-3-carboxylate | 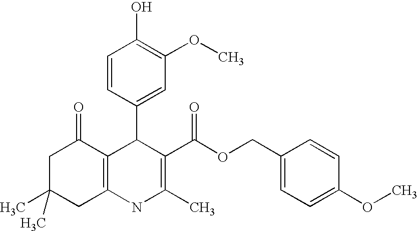 | 30.10402677 | 32.17785865 |
| ST017026 | 2-methylpropyl 7-(4-chlorophenyl)- 2-methyl-5-oxo-4-(2- propoxyphenyl)-1,4,6,7,8- pentahydroquinoline-3-carboxylate | 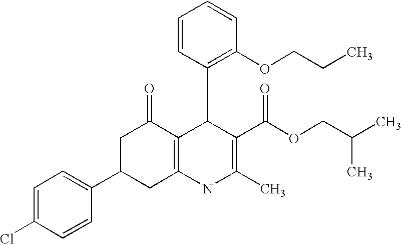 | 40.58672465 | 80.58036883 |
| ST017027 | 2-methoxyethyl 2-methyl-4-(4- nitrophenyl)-5-oxo-7-phenyl- 1,4,6,7,8-pentahydroquinoline-3- carboxylate | 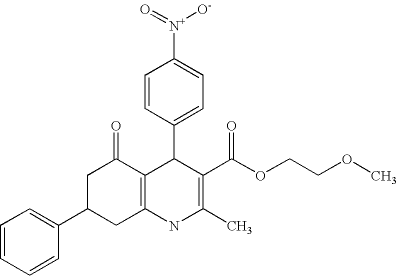 | 35.05227702 | 82.13179062 |
| ST017038 | 2-methyipropyl 7-(4-chlorophenyl)- 4-(2-ethoxyphenyl)-2-methyl-5- oxo-1,4,6,7,8- pentahydroquinoline-3-carboxylate | 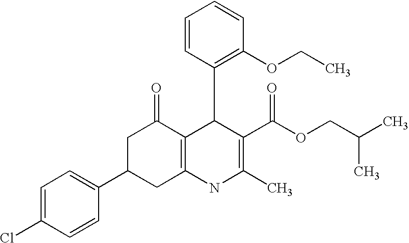 | 42.71052705 | 81.85001064 |
| ST017045 | 4-(2-bromo-4,5-dimethoxyphenyl)- 7-(4-chlorophenyl)-2-methyl-5- oxo-1,4,6,7,8- pentahydroquinoline- 3-carbonitrile | 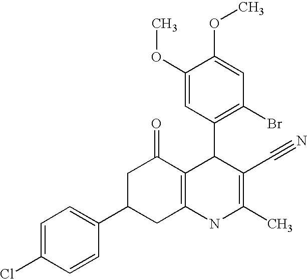 | 42.55374251 | 45.98126952 |
| ST017050 | cyclohexyl 4-(3-chloro-4-hydroxy- 5-methoxyphenyl)-2-methyl-5-oxo- 7-phenyl-1,4,6,7,8- pentahydroquinoline-3-carboxylate | 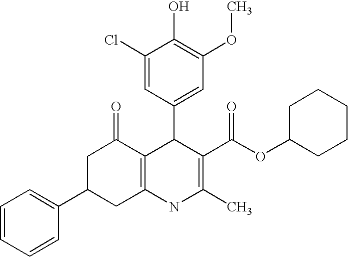 | 39.64973931 | 22.24049078 |
| ST017054 | (4-methoxyphenyl)methyl 2,7,7- trimethyl-4-(6-nitro(2H- benzo[d]1,3-dioxolan-5-yl))-5-oxo- 1,4,6,7,8-pentahydroquinoline-3- carboxylate | 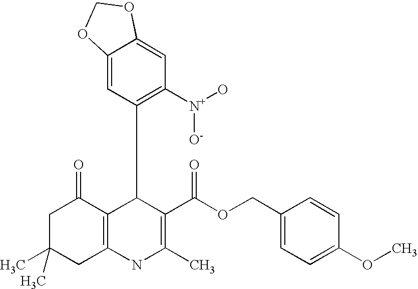 | 46.5375585 | 9.539176868 |
| ST017060 | 2-methoxyethyl4-(3-bromo-5- ethoxy-4-hydroxyphenyl)-2- methyl-5-oxo-7-phenyl-1,4,6,7,8- pentahydroquinoline-3-carboxylate | 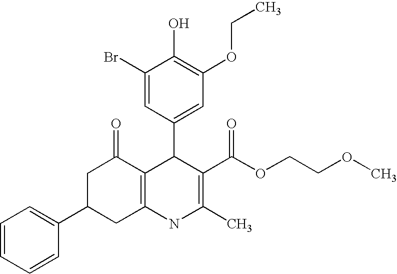 | 38.96351619 | 61.68043948 |
| ST017066 | methylethyl 4-(4-fluorophenyl)-7- (4-methoxyphenyl)-2-methyl-5- oxo-1,4,6,7,8- pentahydroquinoline- 3-carboxylate | 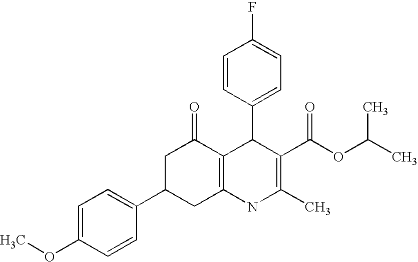 | 24.86267784 | 57.35636803 |
| ST017070 | 2-methylpropyl 4,7-bis(4- chlorophenyl)-2-methyl-5-oxo- 1,4,6,7,8-pentahydroquinoline-3- carboxylate | 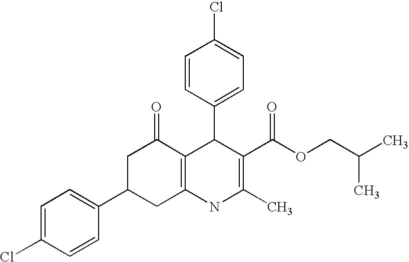 | 55.42134154 | 89.11011598 |
| ST017078 | ethyl 7-(4-methoxyphenyl)-2- methyl-5-oxo-4-[4- (phenylmethoxy)phenyl]-1,4,6,7,8- pentahydroquinoline-3-carboxylate | 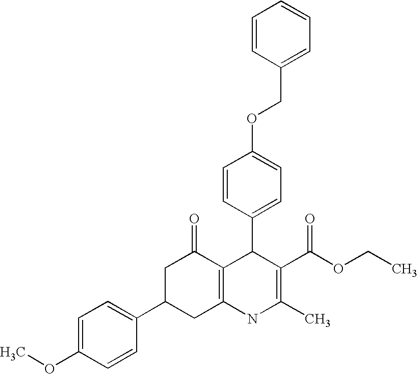 | 51.81160551 | 49.37881673 |
| ST017084 | 2-phenoxyethyl 2-methyl-5-oxo-7- phenyl-4-(4-pyridyl)-1,4,6,7,8- pentahydroquinoline-3-carboxylate | 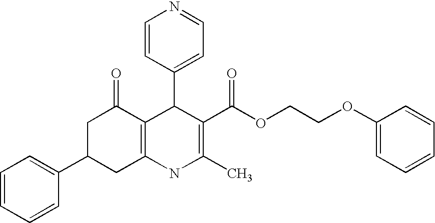 | 54.26688983 | 84.68712533 |
| ST017089 | methylpropyl 4,7-bis(4- chlorophenyl)-2-methyl-5-oxo- 1,4,6,7,8-pentahydroquinoline-3- carboxylate | 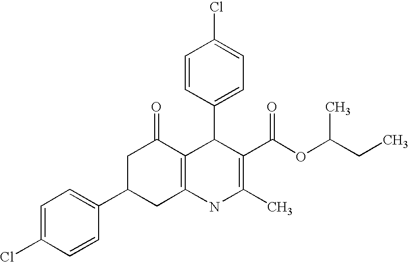 | 26.87153374 | 82.52158164 |
| ST017091 | methylpropyl 7-(4-chlorophenyl)-2- methyl-5-oxo-4-(3,4,5- trimethoxyphenyl)-1,4,6,7,8- pentahydroquinoline-3-carboxylate | 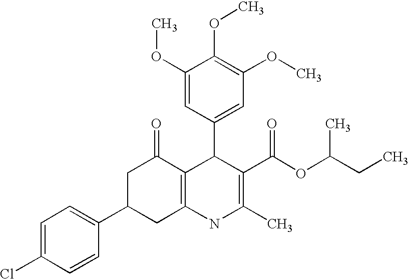 | 65.65736656 | 91.03019648 |
| ST017092 | methylpropyl 4-(4-ethylphenyl)-7- (4-methoxyphenyl)-2-methyl-5- oxo-1,4,6,7,8- pentahydroquinoline- 3-carboxylate | 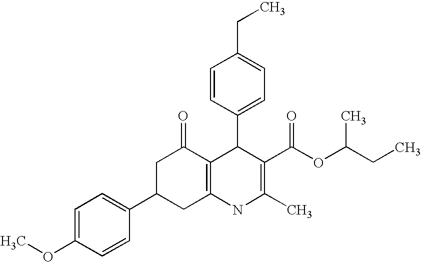 | 41.34196678 | 30.88880794 |
| ST017093 | methylpropyl 7-(4-chlorophenyl)-2- methyl-5-oxo-4-(4-propoxyphenyl)- 1,4,6,7,8-pentahydroquinoline-3- carboxylate | 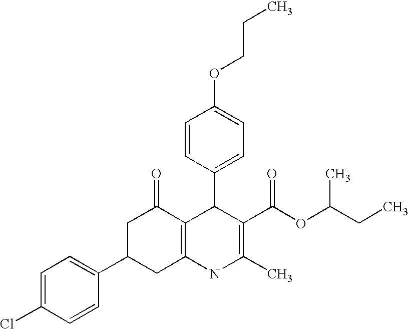 | 46.78699593 | 94.96982242 |
| ST017094 | 2-methoxyethyl 4-(2H-benzo[3,4- d]1,3-dioxolen-5-yl)-7-(4- methoxyphenyl)-2-methyl-5-oxo- 1,4,6,7,8-pentahydroquinoline-3- carboxylate | 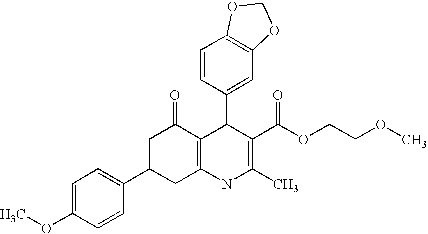 | 68.89519298 | 73.76168695 |
| ST017095 | methylpropyl 4-(3,4- dichlorophenyl)-7-(4- chlorophenyl)-2-methyl-5-oxo- 1,4,6,7,8-pentahydroquinoline-3- carboxylate | 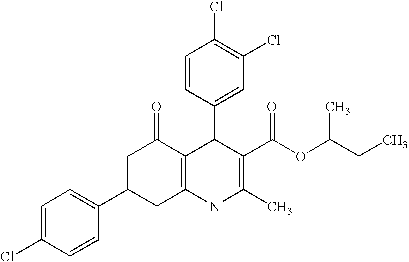 | 61.27785421 | 37.14055748 |
| ST017097 | 2-methoxyethyl 4-(3- methoxyphenyl)-7-(4- methoxyphenyl)-2-methyl-5-oxo- 1,4,6,7,8-pentahydroquinoline-3- carboxylate | 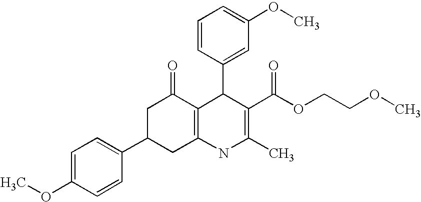 | 68.17903292 | 80.92025428 |
| ST017099 | 2-methoxyethyl 4-(4- bromophenyl)-7-(4- methoxyphenyl)-2-methyl-5-oxo- 1,4,6,7,8-pentahydroquinoline-3- carboxylate | 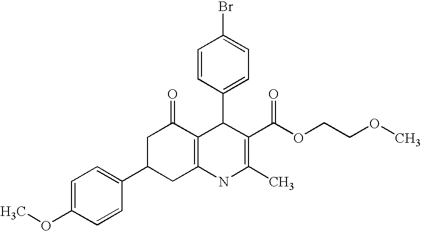 | 84.49922696 | 56.98584879 |
| ST017106 | methyl 4-{3-[(2- ethylthioethyl)oxycarbonyl]-2- methyl-5-oxo-4-1,4,6,7,8- pentahydroquinolyl}benzoate | 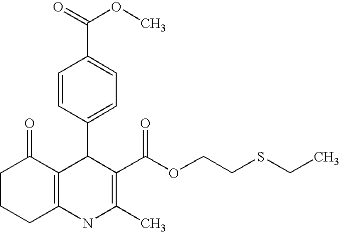 | 36.83309457 | 0 |
| ST017113 | 2-ethylthioethyl 4-[4-(2- hydroxyethoxy)-3-methoxyphenyl]- 2-methyl-5-oxo-1,4,6,7,8- pentahydroquinoline-3-carboxylate | 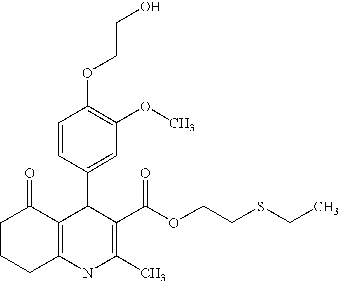 | 82.57372236 | 74.11068674 |
| ST017116 | 2-ethylthioethyl 4-(3-methoxy-4- propoxyphenyl)-2,7,7-trimethyl-5- oxo-1,4,6,7,8- pentahydroquinoline-3-carboxylate | 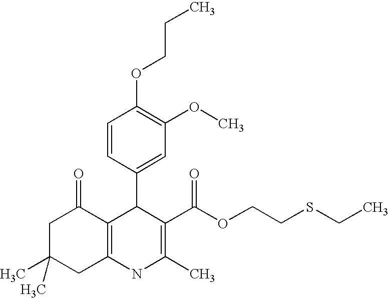 | 43.53427611 | 43.00724393 |
| ST017117 | 2-methylpropyl 7-(4-chlorophenyl)- 4-(3-ethoxy-4-hydroxyphenyl)-2- methyl-5-oxo-1,4,6,7,8- pentahydroquinoline-3-carboxylate | 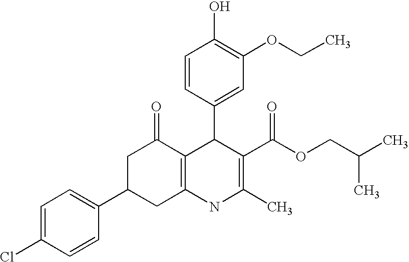 | 25.71963518 | 81.72120879 |
| ST017133 | phenylmethyl 4-(2H-benzo[3,4- d]1,3-dioxolen-5-yl)-7-(4- methoxyphenyl)-2-methyl-5-oxo- 1,4,6,7,8-pentahydroquinoline-3- carboxylate | 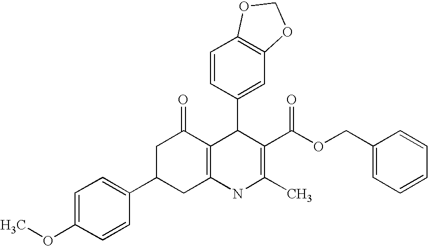 | 39.21220976 | 79.19910365 |
| ST017136 | ethyl 4,7-bis(4-methoxyphenyl)-2- methyl-5-oxo-1,4,6,7,8- pentahydroquinoline-3-carboxylate | 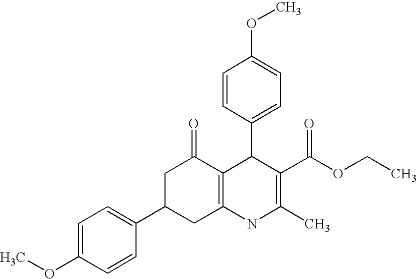 | 87.67237344 | 66.10346826 |
| ST017144 | ethyl 4-(3-chlorophenyl)-7-(4- methoxyphenyl)-2-methyl-5-oxo- 1,4,6,7,8-pentahydroquinoline-3- carboxylate | 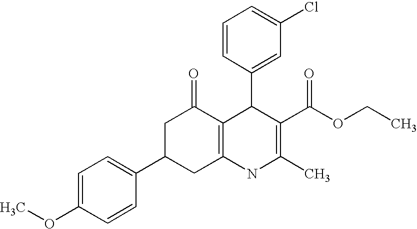 | 23.42051886 | 55.61544295 |
| ST017167 | butyl 4-(4-bromophenyl)-2-methyl- 5-oxo-1,4,6,7,8- pentahydroquinoline-3-carboxylate | 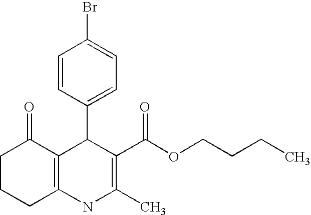 | 79.03484317 | 50.53395172 |
| ST017170 | cyclohexyl 4-(4-bromophenyl)-2- methyl-5-oxo-1,4,6,7,8- pentahydroquinoline-3-carboxylate | 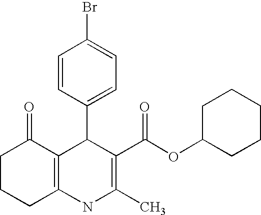 | 3.468382474 | 1.380068974 |
| ST017171 | cyclopentyl 2-methyl-5-oxo-4-(2- thienyl)-1,4,6,7,8- pentahydroquinoline-3-carboxylate | 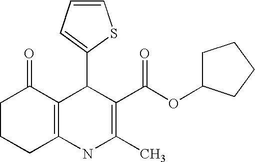 | 62.5408814 | 63.41479367 |
| ST017175 | butyl 4-(4-chlorophenyl)-2-methyl- 5-oxo-1,4,6,7,8- pentahydroquinoline-3-carboxylate | 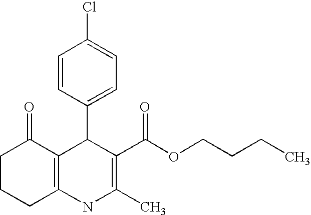 | 59.76125573 | 34.51599477 |
| ST017176 | cyclopentyl 4-(2-methoxyphenyl)- 2-methyl-5-oxo-7-phenyl-1,4,6,7,8- pentahydroquinoline-3-carboxylate | 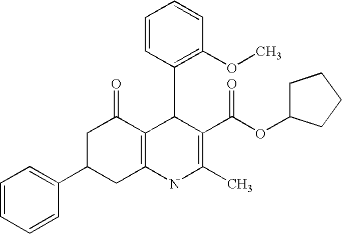 | 34.20809985 | 49.78415983 |
| ST017183 | propyl 4-(3,4-dichlorophenyl)-7-(4- chlorophenyl)-2-methyl-5-oxo- 1,4,6,7,8-pentahydroquinoline-3- carboxylate | 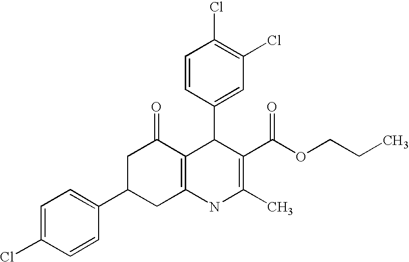 | 50.10659331 | 67.88024735 |
| ST017199 | 2-ethylthioethyl 4-(2- bromophenyl)-2-methyl-5-oxo- 1,4,6,7,8-pentahydroquinoline-3- carboxylate | 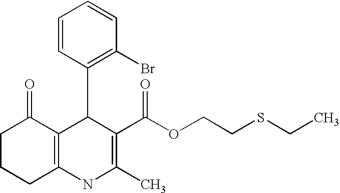 | 47.8978375 | 99.98691878 |
| ST017201 | 2-ethylthioethyl 4-(4- bromophenyl)-2-methyl-5-oxo-7- phenyl-1,4,6,7,8- pentahydroquinoline- 3-carboxylate | 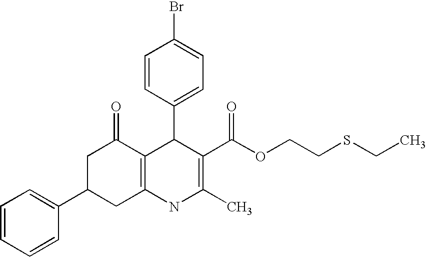 | 57.45410609 | 85.21108336 |
| ST017212 | methylethyl 2-methyl-5-oxo-4,7- diphenyl-1,4,6,7,8- pentahydroquinoline-3-carboxylate | 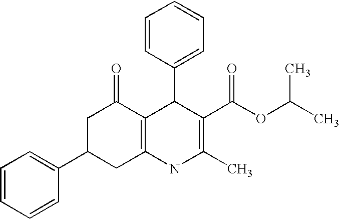 | 25.4069575 | 57.14591509 |
| ST017213 | methylethyl 4-(4-chlorophenyl)- 2,7,7-trimethyl-5-oxo-1,4,6,7,8- pentahydroquinoline-3-carboxylate | 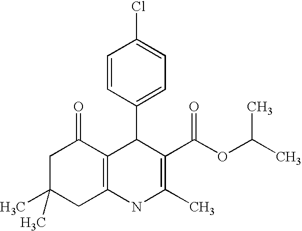 | 44.3672627 | 91.43833987 |
| ST017220 | methylethyl 4-(2-methoxyphenyl)- 2-methyl-5-oxo-7-phenyl-1,4,6,7,8- pentahydroquinoline-3-carboxylate | 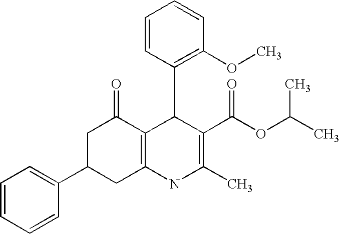 | 60.94615877 | 99.5938875 |
| ST017226 | propyl 4-(2,4-dimethoxyphenyl)- 2,7,7-trimethyl-5-oxo-1,4,6,7,8- pentahydroquinoline-3-carboxylate | 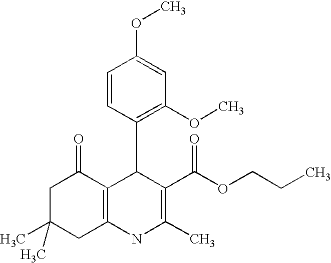 | 23.5578961 | 26.46509692 |
| ST017228 | phenylmethyl 4-(3-ethoxy-4- hydroxyphenyl)-2-methyl-5-oxo-7- phenyl-1,4,6,7,8- pentahydroquinoline- 3-carboxylate | 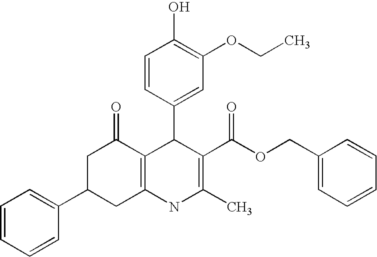 | 95.63601709 | 60.15875847 |
| ST017229 | methylethyl 4-(2,4- dimethoxyphenyl)-2-methyl-5-oxo- 7-phenyl-1,4,6,7,8- pentahydroquinoline- 3-carboxylate | 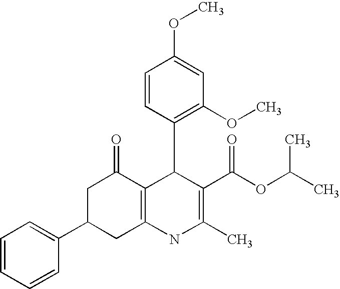 | 63.05280778 | 98.67522892 |
| ST017230 | 4-{2-methyl-5-oxo-7-phenyl-3-[(2- phenoxyethyl)oxycarbonyl]-4- 1,4,6,7,8- pentahydroquinolyl}phenyl acetate | 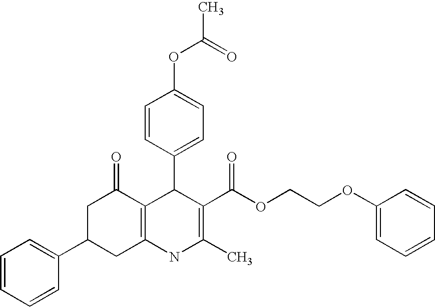 | 69.12491375 | 84.67891545 |
| ST017233 | phenylmethyl 2-methyl-5-oxo-4-(2- thienyl)-1,4,6,7,8- pentahydroquinoline-3-carboxylate | 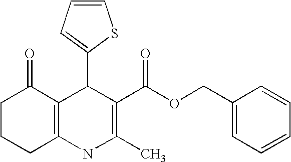 | 60.89928247 | 27.40397193 |
| ST017236 | methylpropyl 7-(4- methoxyphenyl)-2-methyl-4-(6- nitro(2H-benzo[d]1,3-dioxolan-5- yl))-5-oxo-1,4,6,7,8- pentahydroquinoline-3-carboxylate | 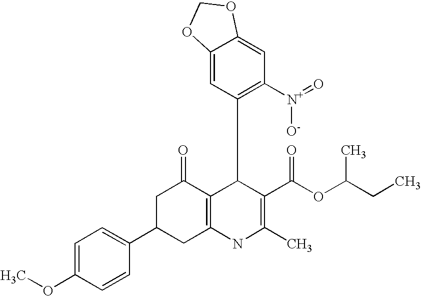 | 60.98461891 | 98.06922004 |
| ST017242 | oxolan-2-ylmethyl 4-(3- chlorophenyl)-2,7,7-trimethyl-5- oxo-1,4,6,7,8- pentahydroquinoline- 3-carboxylate | 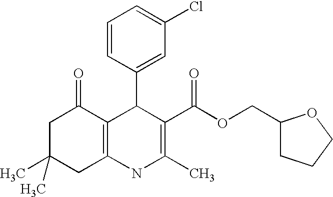 | 49.55527527 | 0 |
| ST017243 | 2-ethylthioethyl 7-(4- chlorophenyl)-4-(3-ethoxy-4- hydroxyphenyl)-2-methyl-5-ox o- 1,4,6,7,8-pentahydroquinoline-3- carboxylate | 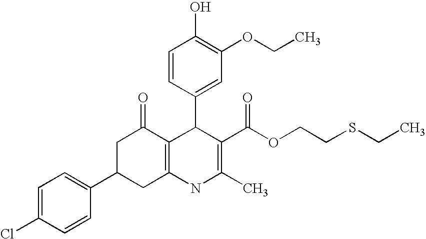 | 69.61170535 | 15.61606433 |
| ST017244 | phenylmethyl 7-(4- methoxyphenyl)-2-methyl-5-oxo-4- (3,4,5-trimethoxyphenyl)-1,4,6,7,8- pentahydroquinoline-3- carboxylate | 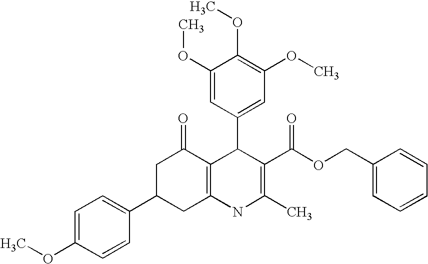 | 65.41671444 | 24.37973506 |
| ST017249 | propyl 2,7,7-trimethyl-5-oxo-4-(4- pyridyl)-1,4,6,7,8- pentahydroquinoline-3-carboxylate | 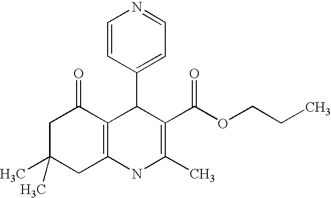 | 66.9793936 | 75.13869511 |
| ST017250 | propyl 2-methyl-5-oxo-4-(3- pyridyl)-1,4,6,7,8- pentahydroquinoline-3-carboxylate | 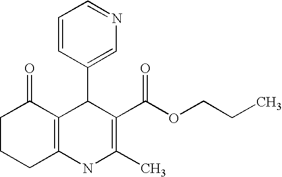 | 28.45419253 | 49.21913677 |
| ST017251 | ethyl 2-methyl-5-oxo-7-phenyl-4- (2-pyridyl)-1,4,6,7,8- pentahydroquinoline-3-carboxylate | 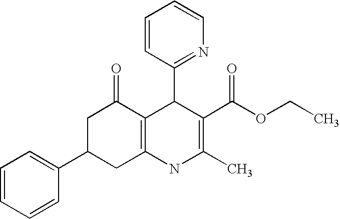 | 90.43637078 | 34.33817904 |
| ST017252 | 2-methoxyethyl 2,7,7-trimethyl-5- oxo-4-(3-pyridyl)-1,4,6,7,8- pentahydroquinoline-3-carboxylate | 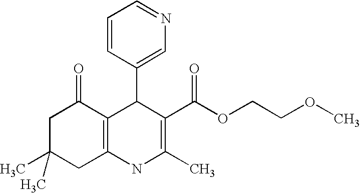 | 89.56698717 | 70.50722355 |
| ST017259 | propyl 4-(4-bromophenyl)-2- methyl-5-oxo-1,4,6,7,8- pentahydroquinoline-3-carboxylate | 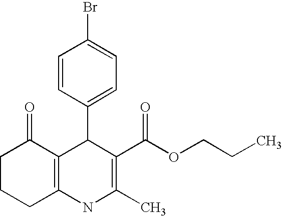 | 65.46995037 | 86.97001065 |
| ST017262 | propyl 2,7,7-trimethyl-5-oxo-4- phenyl-1,4,6,7,8- pentahydroquinoline-3-carboxylate | 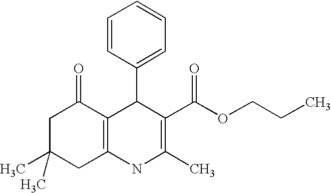 | 60.11933037 | 85.99906987 |
| ST017264 | propyl 4-(4-ethylphenyl)-2-methyl- 5-oxo-7-phenyl-1,4,6,7,8- pentahydroquinoline-3-carboxylate | 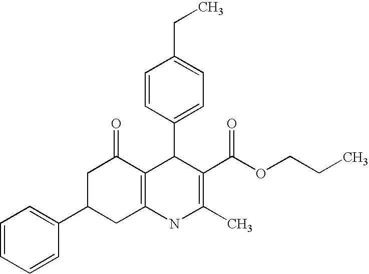 | 29.71383643 | 48.22359242 |
| ST017266 | phenylmethyl 7-(4- methoxyphenyl)-2-methyl-5-oxo-4- (4-pyridyl)-1,4,6,7,8- pentahydroquinoline- 3-carboxylate | 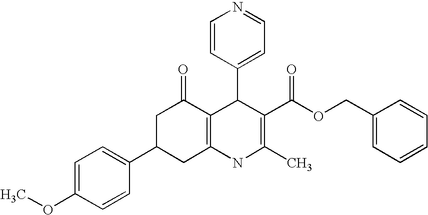 | 65.50598699 | 64.58856534 |
| ST017293 | propyl 4-(2,4-dichlorophenyl)-2- methyl-5-oxo-7-phenyl-1,4,6,7,8- pentahydroquinoline-3-carboxylate | 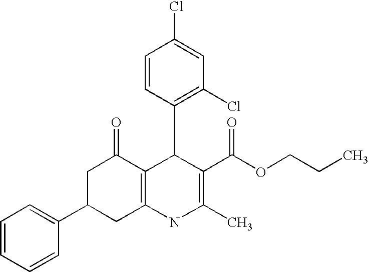 | 57.71634261 | 95.21340594 |
| ST017304 | cyclohexyl 4-(2-ethoxyphenyl)- 2,7,7-trimethyl-5-oxo-1,4,6,7,8- pentahydroquinoline-3-carboxylate | 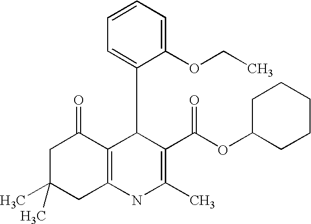 | 51.96645318 | 57.24530057 |
| ST017312 | 2-ethoxyethyl 4-(2-fluorophenyl)-2- methyl-5-oxo-7-phenyl-1,4,6,7,8- pentahydroquinoline-3-carboxylate | 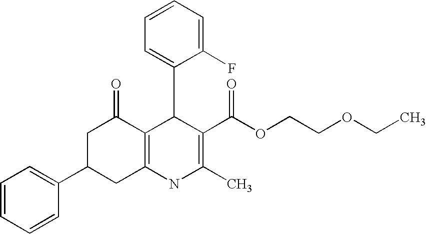 | 15.34382218 | 71.23632927 |
| ST017313 | 2-ethoxyethyl 4-(4-bromophenyl)- 2-methyl-5-oxo-7-phenyl-1,4,6,7,8- pentahydroquinoline-3-carboxylate | 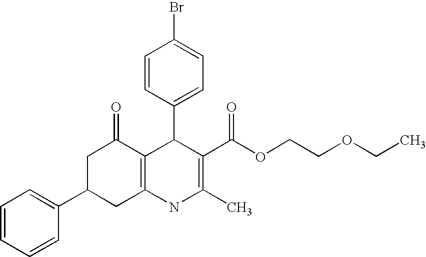 | 44.23209225 | 85.4427892 |
| ST017315 | 2-phenoxyethyl 4-(3- hydroxyphenyl)-2-methyl-5-oxo-7- phenyl-1,4,6,7,8- pentahydroquinoline- 3-carboxylate | 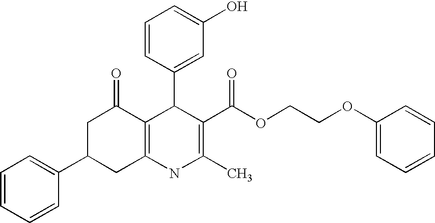 | 43.82586119 | 53.92921974 |
| ST017319 | methylpropyl 4-(3,4- dichlorophenyl)-2-methyl-5-oxo-7- phenyl-1,4,6,7,8- pentahydroquinoline- 3-carboxylate | 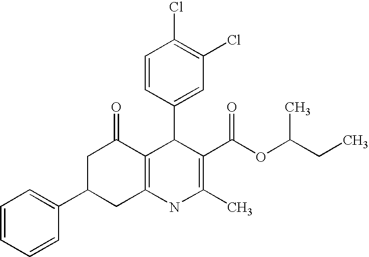 | 26.79773624 | 97.52674138 |
| ST017324 | methylethyl 4-(2,3- dimethoxyphenyl)-7-(4- chlorophenyl)-2-methyl-5-oxo- 1,4,6,7,8-pentahydroquinoline-3- carboxylate | 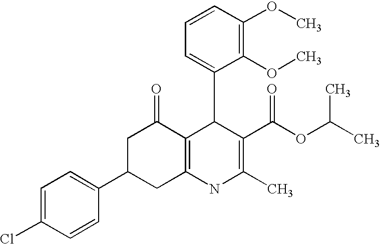 | 30.21651789 | 99.21799131 |
| ST017331 | methylethyl 7-(4-chlorophenyl)-2- methyl-5-oxo-4-(3-pyridyl)- 1,4,6,7,8-pentahydroquinoline-3- carboxylate | 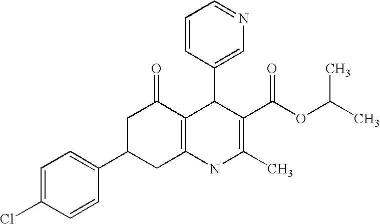 | 1.107133987 | 0 |
| ST017336 | pentyl 4-(3-hydroxyphenyl)-2- methyl-5-oxo-1,4,6,7,8- pentahydroquinoline-3-carboxylate | 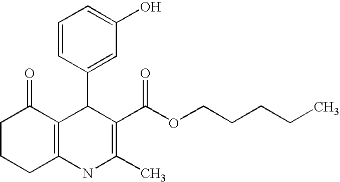 | 62.87916198 | 72.74895647 |
| ST017342 | 2-methylpropyl 2-methyl-5-oxo-4- [4-(phenylmethoxy)phenyl]- 1,4,6,7,8-pentahydroquinoline-3- carboxylate | 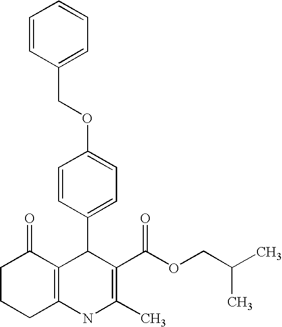 | 28.80345377 | 93.48894 |
| ST018638 | 4-(2,5-dimethoxyphenyl)-2-methyl- 5-oxo-7-phenyl-1,4,6,7,8- pentahydroquinoline-3-carbonitrile | 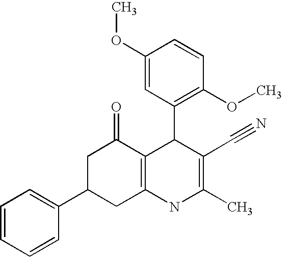 | 45.37496165 | 53.78765937 |
| ST018645 | (4-methoxyphenyl)methyl 4-(4- bromophenyl)-2-methyl-5-oxo- 1,4,6,7,8-pentahydroquinoline-3- carboxylate | 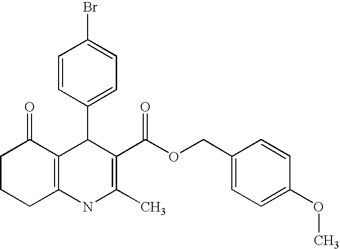 | 7.025874034 | 18.01175569 |
| ST018648 | (4-methoxyphenyl)methyl 4-(4- chlorophenyl)-2-methyl-5-oxo- 1,4,6,7,8-pentahydroquinoline-3- carboxylate | 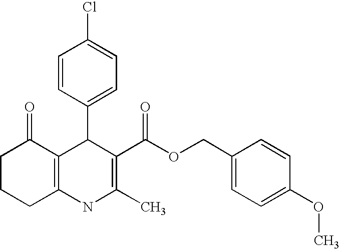 | 7.486522419 | 10.98560354 |
| ST020008 | ethyl 4-(2,4-dichlorophenyl)-2,7,7- trimethyl-5-oxo-1,4,6,7,8- pentahydroquinoline-3-carboxylate | 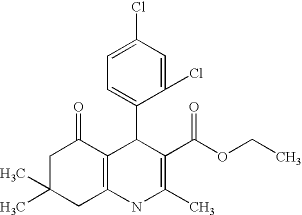 | 61.71899426 | 95.6946929 |
| ST020910 | ethyl 4-(4-bromo-5-methyl(2- thienyl))-2-methyl-5-oxo-1,4,6,7,8- pentahydroquinoline-3-carboxylate | 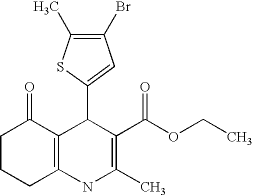 | 47.34597572 | 69.8003805 |
| ST024431 | butyl 4-(4-chlorophenyl)-2,7,7- trimethyl-5-oxo-1,4,6,7,8- pentahydroquinoline-3-carboxylate | 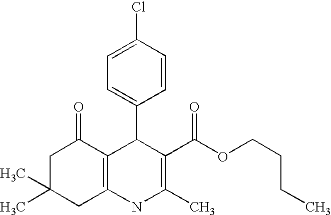 | 0 | 0 |
| ST024440 | ethyl 4-(4-chlorophenyl)-2-methyl- 5-oxo-1,4,6,7,8- pentahydroquinoline-3-carboxylate | 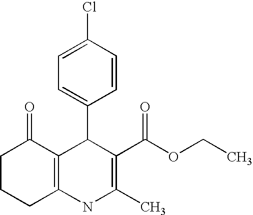 | 63.06565518 | 97.73536632 |
| ST024444 | ethyl 4-(2,4-dichlorophenyl)-2- methyl-5-oxo-1,4,6,7,8- pentahydroquinoline-3-carboxylate | 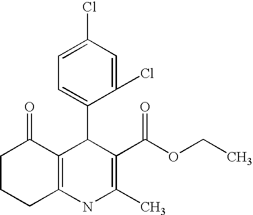 | 37.43806116 | 16.59578882 |
| ST024487 | cycloheptyl 4-(3-bromophenyl)- 2,7,7-trimethyl-5-oxo-1,4,6,7,8- pentahydroquinoline-3-carboxylate | 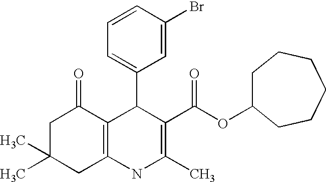 | 5.065882814 | 3.7720845 |
| ST024504 | cycloheptyl 2,7,7-trimethyl-5-oxo- 4-(2,3,4-trimethoxyphenyl)- 1,4,6,7,8-pentahydroquinoline-3- carboxylate | 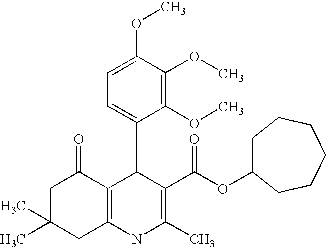 | 0.415307894 | 0 |
| ST024574 | methyl 7-(3,4-dimethoxyphenyl)-2- methyl-5-oxo-4-(3,4,5- trimethoxyphenyl)-1,4,6,7,8- pentahydroquinoline-3-carboxylate | 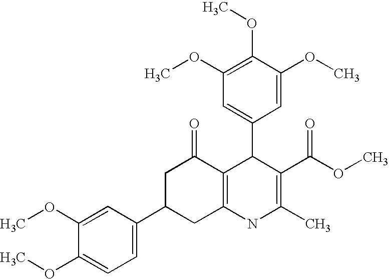 | 65.26567529 | 83.64969056 |
| ST028547 | methyl 4-(4-bromophenyl)-2- methyl-5-oxo-1,4,6,7,8- pentahydroquinoline-3-carboxylate | 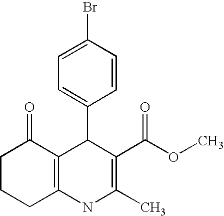 | 72.08053638 | 45.31341839 |
| ST028591 | ethyl 4-{4-[(2- chlorophenyl)methoxy]-3- methoxyphenyl}-2-methyl-5-oxo- 1,4,6,7,8-pentahydroquinoline-3- carboxylate | 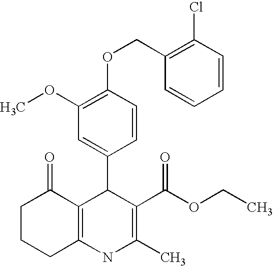 | 98.28146176 | 69.5640148 |
| ST028592 | methyl 4-{4-[(3- chlorophenyl)methoxy]-3- methoxyphenyl}-2,7,7-trimethyl-5- oxo-1,4,6,7,8- pentahydroquinoline-3-carboxylate | 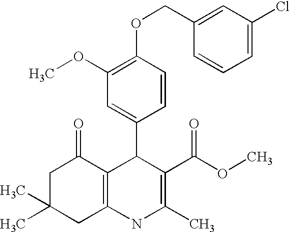 | 28.31808795 | 97.06716982 |
| ST028595 | methyl 4-{4-[(4- chlorophenyl)methoxy]-3- methoxyphenyl}-2,7,7-trimethyl-5- oxo-1,4,6,7,8- pentahydroquinoline-3-carboxylate | 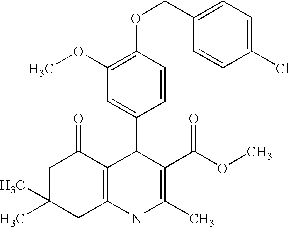 | 34.41174592 | 98.90009509 |
| ST028598 | ethyl 4-{4-[(4- chlorophenyl)methoxy]-3- methoxyphenyl}-2-methyl-5-oxo- 1,4,6,7,8 -pentahydroquinoline-3- carboxylate | 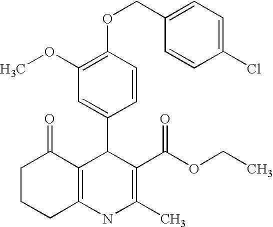 | 27.41755785 | 71.56195482 |
| ST028606 | ethyl 4-{3-methoxy-4-[(4- methylphenyl)methoxy]phenyl}-2- methyl-5-oxo-1,4,6,7,8- pentahydroquinoline-3-carboxylate | 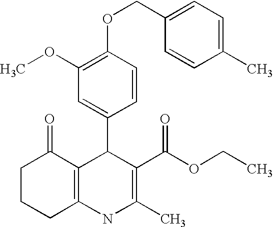 | 24.21851161 | 51.9808022 |
| ST028607 | methyl 4-(3-methoxy-4-{[3- (trifluoromethyl)phenyl]methoxy} phenyl)-2,7,7-trimethyl-5-oxo- 1,4,6,7,8-pentahydroquinoline-3- carboxylate | 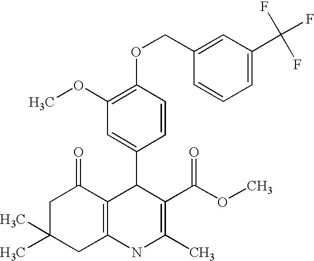 | 23.78243931 | 63.29282628 |
| ST040993 | 2-amino-1-(4-fluorophenyl)-5-oxo- 7-phenyl-4-(3-thienyl)-1,4,6,7,8- pentahydroquinoline-3-carbonitrile | 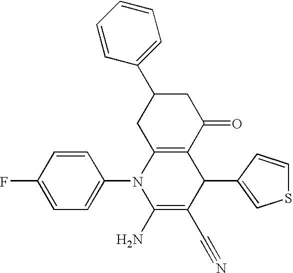 | 65.71884708 | 46.89023024 |
| ST041668 | 5-(4-fluorophenyl)-4,8-dioxo- 1,2,3,5,7,9,10,11,12,13,15- undecahydrocyclohepta[1,2- b]quinolino[1′,2′- 2,1]pyrimidino[5,6-d]thiophene-6- carbonitrile | 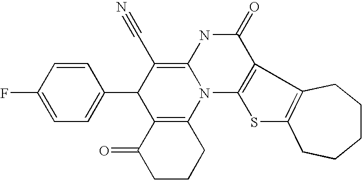 | 88.72375581 | 27.87197958 |
| ST045143 | methyl 4-(4-bromo-2,5- dimethoxyphenyl)-2-methyl-5-oxo- 7-(2-thienyl)-1,4,6,7,8- pentahydroquinoline-3-carboxylate | 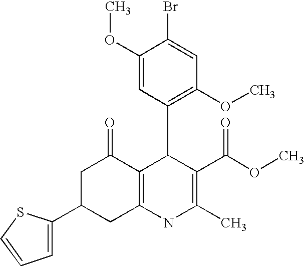 | 22.85871287 | 16.01058456 |
| ST046868 | 2-amino-4-(5-bromo(2-thienyl))-1- [2-chloro-5- (trifluoromethyl)phenyl]-7,7- dimethyl-5-oxo-1,4,6,7,8- pentahydroquinoline-3-carbonitrile | 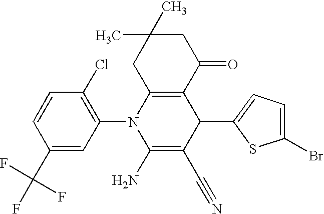 | 29.77636602 | 84.03754489 |
| ST047623 | 2-amino-4-(4-chlorophenyl)-7,7- dimethyl-5-oxo-1-benzyl-1,4,6,7,8- pentahydroquinoline-3-carbonitrile | 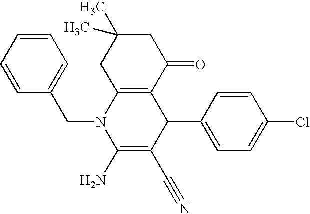 | 95.44262887 | 44.40610034 |
| ST050407 | N-(4-bromophenyl)-2-[4-(2- chlorophenyl)-3-cyano-5-oxo(2- 1,4,6,7,8- pentahydroquinolylthio)]acetamide | 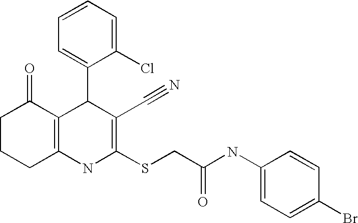 | 56.71913217 | 44.28752729 |
| ST051161 | N-(3-chloro-2-methylphenyl)-2-(3- cyano-7,7-dimethyl-5-oxo-4-(2- thienyl)(2-1,4,6,7,8- pentahydroquinolylthio))acetamide | 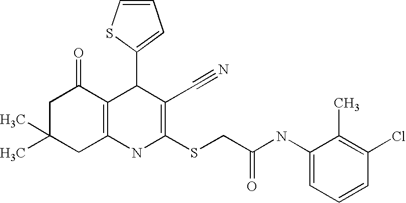 | 12.98647518 | 25.37366361 |
| ST051557 | cyclopentyl 2-methyl-5-oxo-7-(2- thienyl)-4-(2,4,5- trimethoxyphenyl)-1,4,6,7,8- pentahydroquinoline-3-carboxylate | 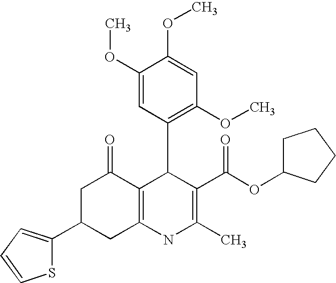 | 57.31234626 | 93.1349412 |
| ST051566 | 2-methylpropyl 4-(2-fluorophenyl)- 2-methyl-5-oxo-7-(2-thienyl)- 1,4,6,7,8-pentahydroquinoline-3- carboxylate | 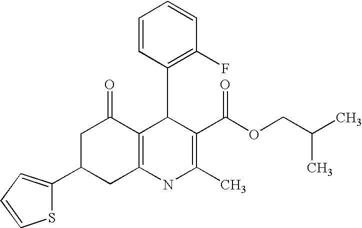 | 63.386505 | 96.06415423 |
| ST051567 | cyclopentyl 2-methyl-4-(5- methyl(2-furyl))-5-oxo-7-(2- thienyl)-1,4,6,7,8- pentahydroquinoline-3-carboxylate | 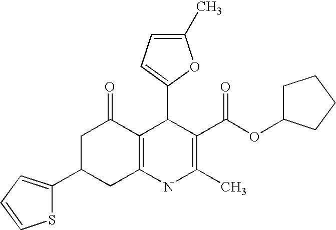 | 60.41444083 | 84.9935221 |
| ST051570 | 2-ethylthioethyl 4-(3- chlorophenyl)-2-methyl-5-oxo-7- (2-thienyl)-1,4,6,7,8- pentahydroquinoline-3-carboxylate | 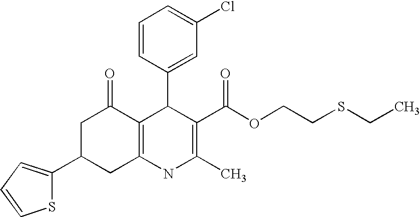 | 31.74108215 | 93.76903374 |
| ST051571 | cyclopentyl 2-methyl-5-oxo-7-(2- thienyl)-4-(2,3,4- trimethoxyphenyl)-1,4,6,7,8- pentahydroquinoline-3-carboxylate | 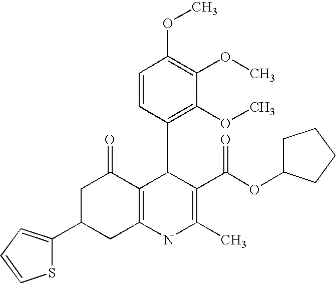 | 40.40342551 | 62.05920904 |
| ST051818 | oxolan-2-ylmethyl 4-(3- chlorophenyl)-2-methyl-5-oxo-7- (2-thienyl)-1,4,6,7,8- pentahydroquinoline-3-carboxylate | 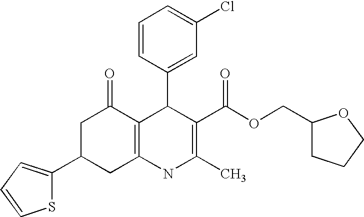 | 37.02511988 | 72.9336363 |
| ST051958 | 2-amino-1-(2-chlorophenyl)-4-(3- hydroxy-4-methoxyphenyl)-5-oxo- 1,4,6,7,8-pentahydroquinoline-3- carbonitrile | 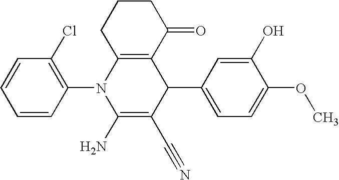 | 56.63072196 | 80.22743306 |
| ST051960 | 2-amino-1-(2,6-difluorophenyl)-5- oxo-4-(3-thienyl)-1,4,6,7,8- pentahydroquinoline-3-carbonitrile | 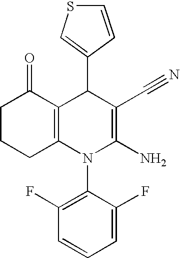 | 48.77920508 | 86.27816587 |
| ST052026 | 2-amino-4-[5-(tert-butyl)(2- thienyl)]-1-(4-bromophenyl)-7,7- dimethyl-5-oxo-1,4,6,7,8- pentahydroquinoline-3-carbonitrile | 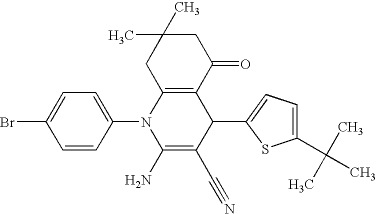 | 13.47181156 | 25.25628719 |
| ST052048 | 2-amino-4-(2,4-dichlorophenyl)-1- (4-bromo-2-fluorophenyl)-7,7- dimethyl-5-oxo-1,4,6,7,8- pentahydroquinoline-3-carbonitrile | 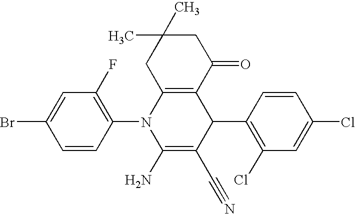 | 58.87022635 | 33.02478711 |
| ST052173 | cyclopentyl 4-(9-ethylcarbazol-3- yl)-2,7,7-trimethyl-5-oxo-1,4,6,7,8- pentahydroquinoline-3-carboxylate | 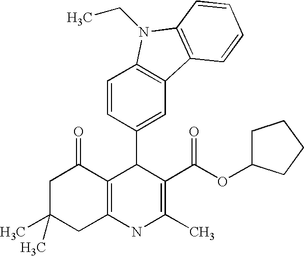 | 64.69964635 | 32.49464416 |
| ST052207 | oxolan-2-ylmethyl 4-(9- ethylcarbazol-3-yl)-2-methyl-5- oxo-1,4,6,7,8- pentahydroquinoline-3-carboxylate | 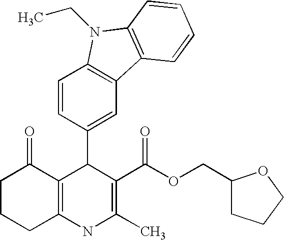 | 52.33723425 | 59.6367503 |
| ST052219 | 2-phenoxyethyl 4-(2- bromophenyl)-2-methyl-5-oxo- 1,4,6,7,8-pentahydroquinoline-3- carboxylate | 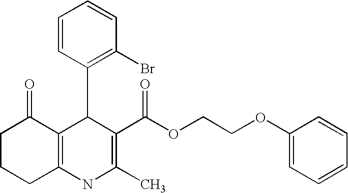 | 14.02589013 | 24.66723947 |
| ST052221 | 2-phenylethyl 4-(3-bromophenyl)- 2-methyl-5-oxo-1,4,6,7,8- pentahydroquinoline-3-carboxylate | 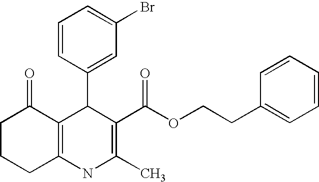 | 62.4228721 | 46.11550641 |
| ST052224 | oxolan-2-ylmethyl 4-(5-bromo-2- methoxyphenyl)-2-methyl-5-oxo- 1,4,6,7,8-pentahydroquinoline-3- carboxylate | 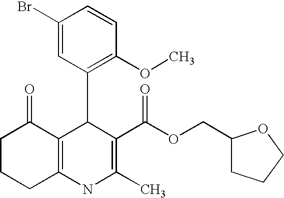 | 76.93285628 | 68.09608191 |
| ST052225 | 2-phenylethyl 4-(2-fluorophenyl)- 2,7,7-trimethyl-5-oxo-1,4,6,7,8- pentahydroquinoline-3-carboxylate | 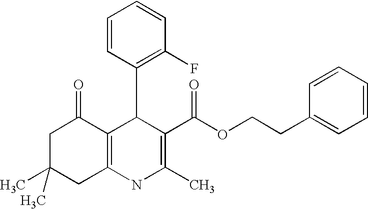 | 37.71751104 | 29.68237443 |
| ST052228 | 2-phenylethyl 2-methyl-5-oxo-4-[2- (trifluoromethyl)phenyl]-1,4,6,7,8- pentahydroquinoline-3-carboxylate | 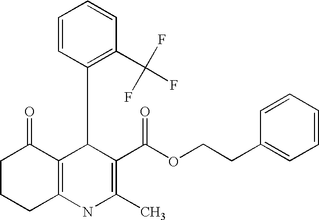 | 60.3610207 | 36.85619602 |
| ST052231 | 2-phenylethyl 4-[4- (dimethylamino)phenyl]-2,7,7- trimethyl-5-oxo-1,4,6,7,8- pentahydroquinoline-3-carboxylate | 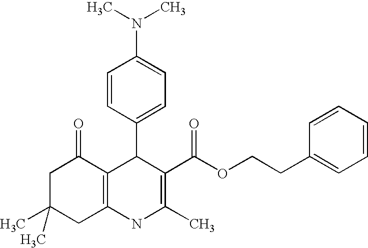 | 38.78591365 | 63.51753527 |
| ST052247 | 2-phenoxyethyl 2-methyl-4-[4- (methylethyl)phenyl]-5-oxo- 1,4,6,7,8-pentahydroquinoline-3- carboxylate | 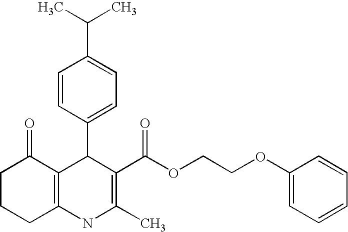 | 97.96630805 | 64.11567858 |
| ST052262 | cyclopentyl 2-methyl-4-(5- methyl(2-thienyl))-5-oxo-1,4,6,7,8- pentahydroquinoline-3-carboxylate | 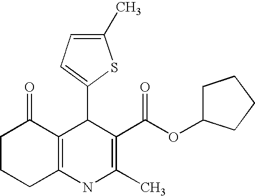 | 13.20949446 | 19.65801715 |
| ST052267 | cyclopentyl 4-(3,4-difluorophenyl)- 2-methyl-5-oxo-1,4,6,7,8- pentahydroquinoline-3-carboxylate | 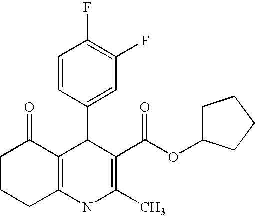 | 50.68946733 | 52.83315426 |
| ST052271 | (4-methoxyphenyl)methyl 2- methyl-5-oxo-4-[2- (trifluoromethyl)phenyl]-1,4,6,7,8- pentahydroquinoline-3-carboxylate | 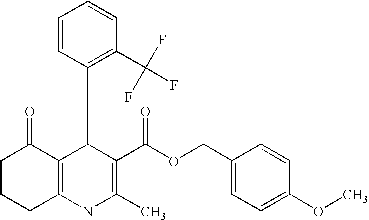 | 25.80335494 | 23.93528692 |
| ST052272 | 2-(methylethoxy)ethyl 2,7,7- trimethyl-4-(4-nitrophenyl)-5-oxo- 1,4,6,7,8-pentahydroquinoline-3- carboxylate | 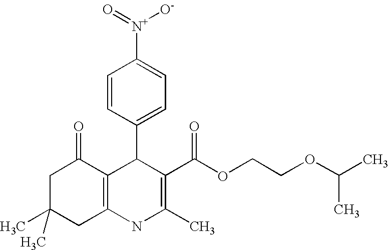 | 65.83638636 | 29.2186425 |
| ST052280 | 2-phenoxyethyl 2-methyl-4-(5- methyl(2-thienyl))-5-oxo-1,4,6,7,8- pentahydroquinoline-3-carboxylate | 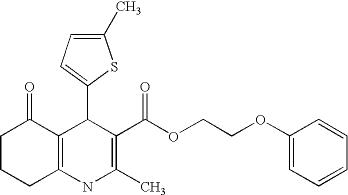 | 73.5680602 | 37.71978644 |
| ST052292 | [4-(4-chlorophenyl)-2-methyl-5- oxo(3-1,4,6,7,8- pentahydroquinolyl)]-N-(2- methoxyphenyl)carboxamide | 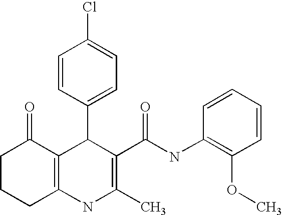 | 85.75817374 | 68.19182074 |
| ST052308 | methyl 2-methyl-4-(5-methyl(2- thienyl))-5-oxo-7-(2-thienyl)- 1,4,6,7,8-pentahydroquinoline-3- carboxylate | 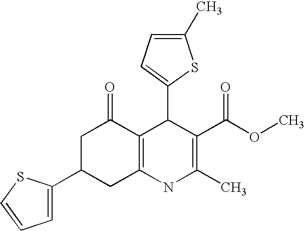 | 48.43761682 | 74.20625461 |
| ST052313 | 2-methoxyethyl 2,7,7-trimethyl-4- [4-(methylethyl)phenyl]-5-oxo- 1,4,6,7,8-pentahydroquinoline-3- carboxylate | 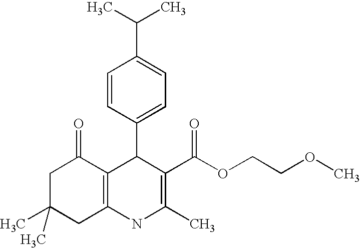 | 26.29321855 | 23.35997928 |
| ST052320 | 2-phenylethyl 2-methyl-5-oxo-4-[4- (trifluoromethyl)phenyl]-1,4,6,7,8- pentahydroquinoline-3-carboxylate | 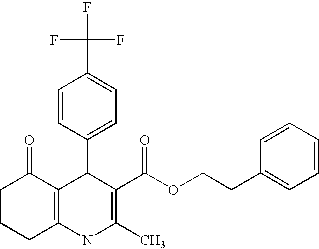 | 9.443192378 | 0 |
| ST052323 | phenylmethyl 2-methyl-5-oxo-4-[4- (trifluoromethyl)phenyl]-1,4,6,7,8- pentahydroquinoline-3-carboxylate | 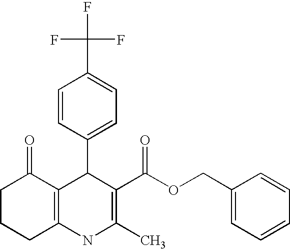 | 4.907047414 | 0 |
| ST052325 | cyclohexyl 4-(3-hydroxy-4- methoxyphenyl)-2,7,7-trimethyl-5- oxo-1,4,6,7,8- pentahydroquinoline-3-carboxylate | 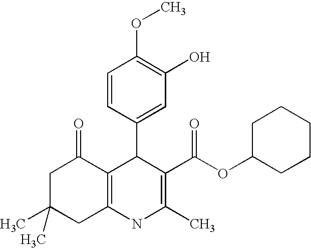 | 41.72492336 | 32.95713848 |
| ST052330 | 2-phenoxyethyl 2-methyl-4-(6- methyl-4-oxochromen-3-yl)-5-oxo- 1,4,6,7,8-pentahydroquinoline-3- carboxylate | 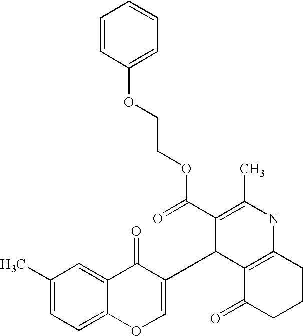 | 42.56005469 | 15.51604302 |
| ST052342 | 2-ethylthioethyl 2,7,7-trimethyl-4- (2-methylphenyl)-5-oxo-1,4,6,7,8- pentahydroquinoline-3-carboxylate | 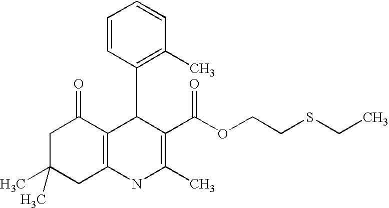 | 13.73300364 | 9.883006394 |
| ST052344 | 2-phenylethyl 2,7,7-trimethyl-4-(5- methyl(2-thienyl))-5-oxo-1,4,6,7,8- pentahydroquinoline-3-carboxylate | 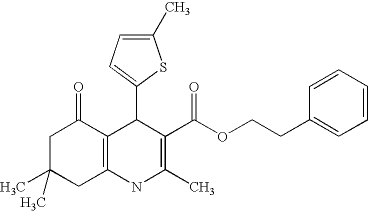 | 24.06700277 | 24.39135774 |
| ST052345 | 2-methylpropyl 4-(3,4- difluorophenyl)-2-methyl-5-oxo- 1,4,6,7,8-pentanydroquinoline-3- carboxylate | 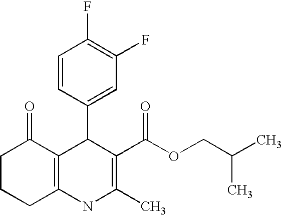 | 43.63551157 | 7.814628893 |
| ST052347 | methyl 2-methyl-5-oxo-4-[3- (trifluoromethyl)phenyl]-1,4,6,7,8- pentahydroquinoline-3-carboxylate | 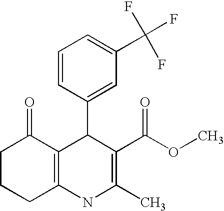 | 57.15903143 | 64.31361292 |
| ST052351 | 2-methylpropyl 4-(2-chloro-5- nitrophenyl)-2-methyl-5-oxo- 1,4,6,7,8-pentahydroquinoline-3- carboxylate | 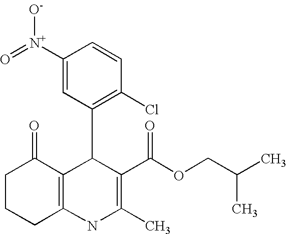 | 19.30975829 | 20.27266892 |
| ST052354 | (4-methoxyphenyl)methyl 2- methyl-4-(3-methylphenyl)-5-oxo- 1,4,6,7,8-pentahydroquinoline-3- carboxylate | 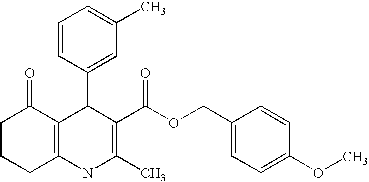 | 95.14929558 | 57.23929616 |
| ST052357 | (4-methoxyphenyl)methyl 4-(4- chloro-3-nitrophenyl)-2,7,7- trimethyl-5-oxo-1,4,6,7,8- pentahydroquinoline-3-carboxylate | 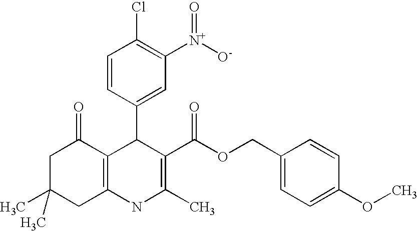 | 13.28519701 | 23.35997928 |
| ST052359 | methyl 2,7,7-trimethyl-5-oxo-4-[4- (trifluoromethyl)phenyl]-1,4,6,7,8- pentahydroquinoline-3-carboxylate | 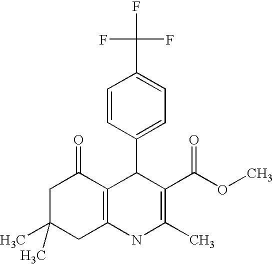 | 30.96795125 | 29.53660736 |
| ST052360 | methyl 2,7,7-trimethyl-5-oxo-4-[3- (phenylmethoxy)phenyl]-1,4,6,7,8- pentahydroquinoline-3-carboxylate | 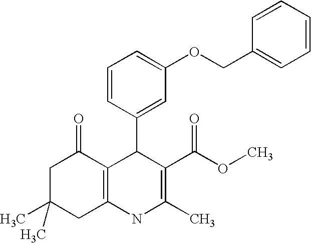 | 55.67061513 | 61.6422263 |
| ST052361 | methyl 4-(4-chloro-3-nitrophenyl)- 2-methyl-5-oxo-1,4,6,7,8- pentahydroquinoline-3-carboxylate | 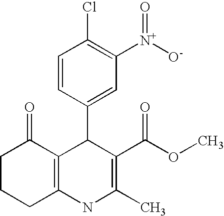 | 84.08150241 | 63.23164915 |
| ST052362 | (4-methoxyphenyl)methyl 2- methyl-5-oxo-4-[4- (trifluoromethyl)phenyl]-1,4,6,7,8- pentahydroquinoline-3-carboxylate | 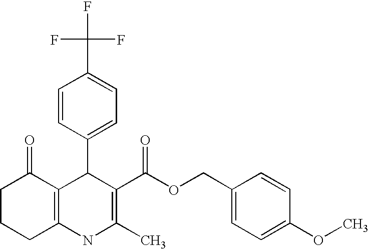 | 7.114037149 | 0 |
| ST052364 | propyl 4-(5-bromo-2- methoxyphenyl)-2-methyl-5-oxo- 1,4,6,7,8-pentahydroquinoline-3- carboxylate | 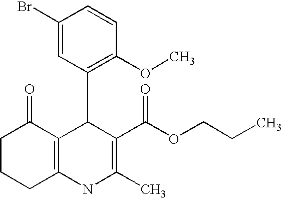 | 27.01642774 | 13.49721803 |
| ST052365 | 2-methylpropyl 2-methyl-4- naphthyl-5-oxo-1,4,6,7,8- pentahydroquinoline-3-carboxylate | 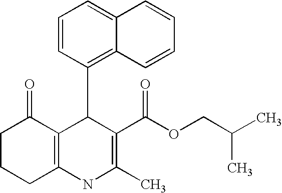 | 59.11246646 | 72.76954338 |
| ST052368 | methyl 4-(2,5-difluorophenyl)-2- methyl-5-oxo-1,4,6,7,8- pentahydroquinoline-3-carboxylate | 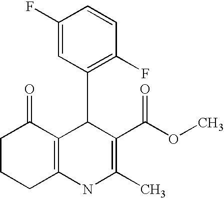 | 66.34915709 | 74.15741075 |
| ST052370 | 2-ethoxyethyl 2-methyl-5-oxo-4-(4- oxochromen-3-yl)-1,4,6,7,8- pentahydroquinoline-3-carboxylate | 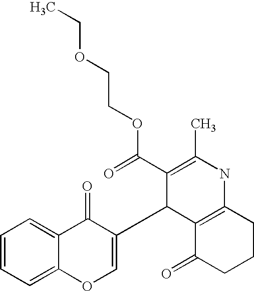 | 68.59445701 | 69.66595611 |
| ST052371 | (4-methoxyphenyl)methyl 2- methyl-5-oxo-4-(2-thienyl)- 1,4,6,7,8-pentahydroquinoline-3- carboxylate | 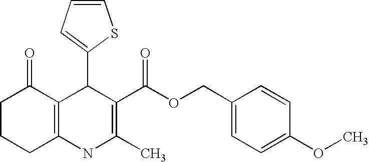 | 59.75365895 | 92.5233839 |
| ST052373 | 2-phenylethyl 4-(2,5- dimethoxyphenyl)-2-methyl-5-oxo- 1,4,6,7,8-pentahydroquinoline-3- carboxylate | 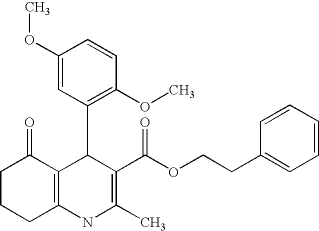 | 39.43108557 | 37.44000798 |
| ST052381 | 2-phenoxyethyl 4-(2- ethoxyphenyl)-2,7,7-trimethyl-5- oxo-1,4,6,7,8- pentahydroquinoline- 3-carboxylate | 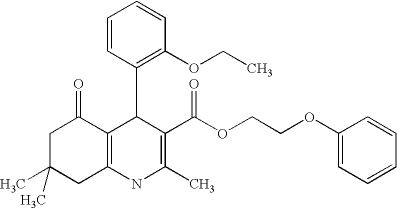 | 45.11735174 | 61.9981966 |
| ST052389 | methyl 4-(2,5-dimethoxyphenyl)-2- methyl-5-oxo-7-(2-thienyl)- 1,4,6,7,8-pentahydroquinoline-3- carboxylate | 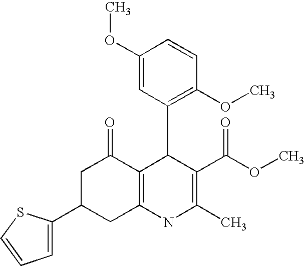 | 78.29681056 | 44.92036384 |
| ST052392 | 2-phenoxyethyl 2,7,7-trimethyl-4- (6-methyl-4-oxochromen-3-yl)-5- oxo-1,4,6,7,8- pentahydroquinoline-3-carboxylate | 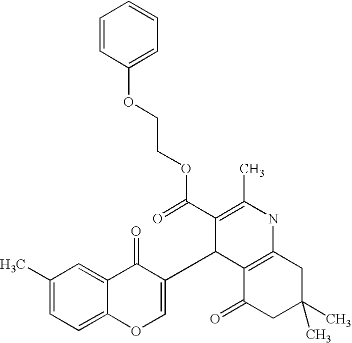 | 28.07507108 | 11.87540774 |
| ST052393 | methyl 2-methyl-5-oxo-7-(2- thienyl)-4-[3- (trifluoromethyl)phenyl]-1,4,6,7,8- pentahydroquinoline-3-carboxylate | 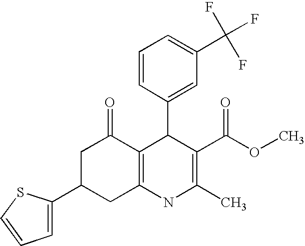 | 34.24523485 | 48.179359 |
| ST052398 | 2-phenylethyl 2,7,7-trimethyl-4-(5- methyl(2-furyl))-5-oxo-1,4,6,7,8- pentahydroquinoline-3-carboxylate | 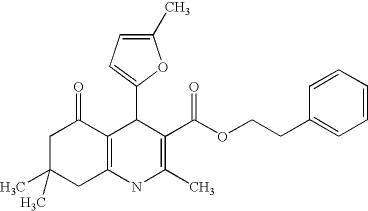 | 44.28019761 | 55.55832575 |
| ST052408 | phenylmethyl 4-(2-bromophenyl)- 2-methyl-5-oxo-1,4,6,7,8- pentahydroquinoline-3-carboxylate | 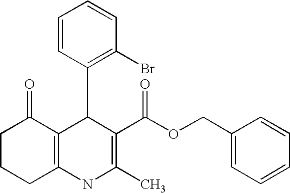 | 62.99228166 | 63.43883619 |
| ST052414 | methyl 4-(3,4-dichlorophenyl)-2- methyl-5-oxo-7-(2-thienyl)- 1,4,6,7,8-pentahydroquinoline-3- carboxylate | 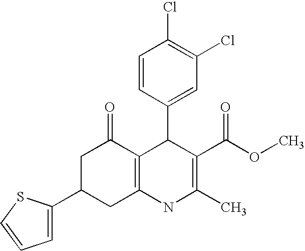 | 70.15989769 | 66.92471027 |
| ST052422 | 2-phenoxyethyl 4-(3-hydroxy-4- methoxyphenyl)-2,7,7-trimethyl-5- oxo-1,4,6,7,8-pentahydroquinoline- 3-carboxylate | 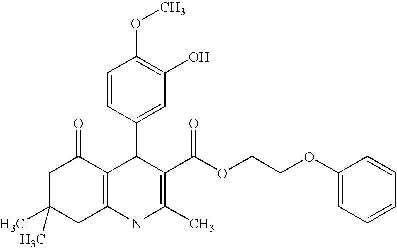 | 38.34653926 | 44.8314406 |
| ST052437 | 2-propoxyethyl 4-(4-bromophenyl)- 2,7,7-trimethyl-5-oxo-1,4,6,7,8- pentahydroquinoline-3-carboxylate | 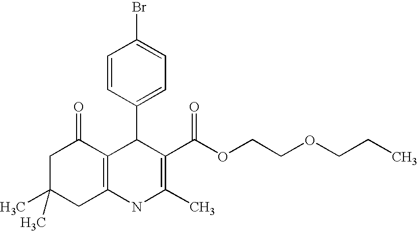 | 5.308983702 | 16.47157573 |
| ST052446 | 2-propoxyethyl 4-(3,4- dichlorophenyl)-2,7,7-trimethyl-5- oxo-1,4,6,7,8- pentahydroquinoline-3-carboxylate | 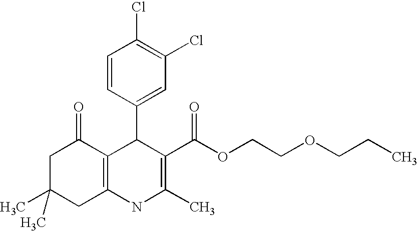 | 58.11351259 | 45.18796461 |
| ST052454 | 2-propoxyethyl 4-(2-chlorophenyl)- 2,7,7-trimethyl-5-oxo-1,4,6,7,8- pentahydroquinoline-3-carboxylate | 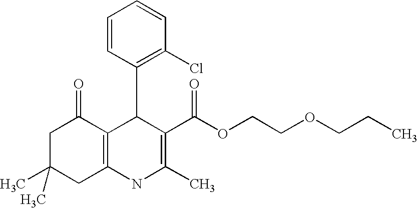 | 25.59167479 | 33.17510998 |
| ST052457 | 2-ethylthioethyl 2,7,7-trimethyl-4- (3-methylphenyl)-5-oxo-1,4,6,7,8- pentahydroquinoline-3-carboxylate | 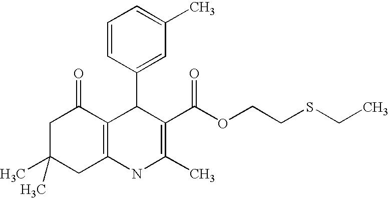 | 38.1018675 | 63.89802834 |
| ST052462 | 2-methoxyethyl 4-(2-bromo-4,5- dimethoxyphenyl)-2-methyl-5-oxo- 1,4,6,7,8-pentahydroquinoline-3- carboxylate | 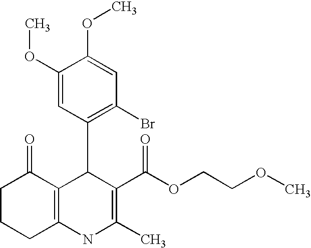 | 61.83051252 | 94.27942282 |
| ST052468 | phenylmethyl 4-(6-chloro-4- oxochromen-3-yl)-2-methyl-5-oxo- 1,4,6,7,8-pentahydroquinoline-3- carboxylate | 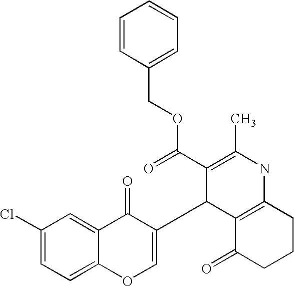 | 43.93110718 | 31.931645 |
| ST052493 | methyl 2-methyl-5-oxo-7-(2- thienyl)-4-[4- (trifluoromethyl)phenyl]-1,4,6,7,8- pentahydroquinoline-3-carboxylate | 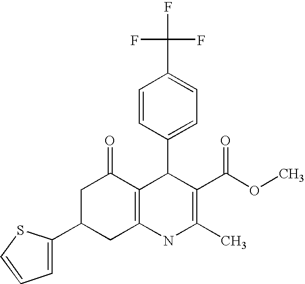 | 39.85078857 | 57.11442672 |
| ST056056 | butyl 4-(3-iodophenyl)-2,7,7- trimethyl-5-oxo-1,4,6,7,8- pentahydroquinoline-3-carboxylate | 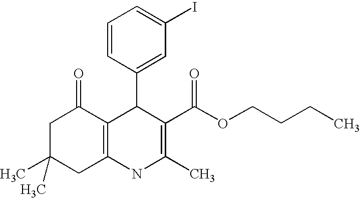 | 6.563115292 | 12.30239302 |
| ST211289 | ethyl 4-(4-bromophenyl)-2-methyl- 5-oxo-1,4,6,7,8- pentahydroquinoline-3-carboxylate | 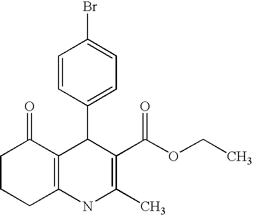 | 89.12708458 | 63.58389823 |
| ST212479 | ethyl 2-methyl-4-(6-nitro(2H- benzo[d]1,3-dioxolan-5-yl))-5-oxo- 1,4,6,7,8-pentahydroquinoline-3- carboxylate | 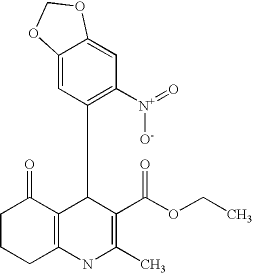 | 60.24790334 | 29.26397197 |
| ST213781 | ethyl 4-(4-ethoxyphenyl)-2-methyl- 5-oxo-1,4,6,7,8- pentahydroquinoline-3-carboxylate | 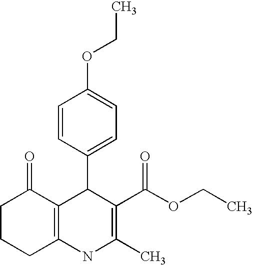 | 66.83271428 | 81.30884997 |
| ST215419 | butyl 4-(3,4-dichlorophenyl)-2,7,7- trimethyl-5-oxo-1,4,6,7,8- pentahydroquinoline-3-carboxylate | 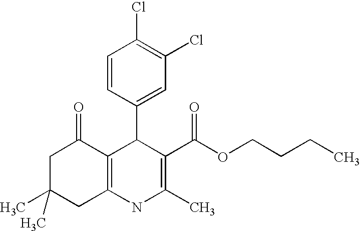 | 0 | 0 |
| ST215449 | methylethyl 4-(3-bromo-4- methoxyphenyl)-2,7,7-trimethyl-5- oxo-1,4,6,7,8- pentahydroquinoline- 3-carboxylate | 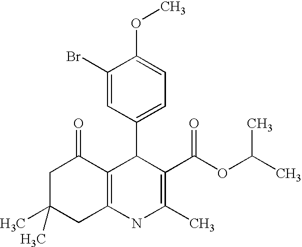 | 24.51134321 | 25.74257426 |
| ST215464 | butyl 2,7,7-trimethyl-4-(2- nitrophenyl)-5-oxo-1,4,6,7,8- pentahydroquinoline-3-carboxylate | 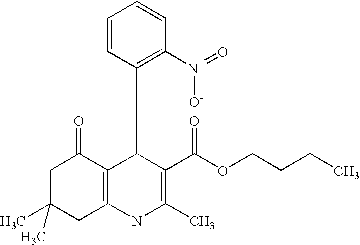 | 62.6959546 | 90.72035885 |
| ST216053 | butyl 2,7,7-trimethyl-4-(6-nitro(2H- benzo[d]1,3-dioxolen-5-yl))-5-oxo- 1,4,6,7,8-pentahydroquinoline-3- carboxylate | 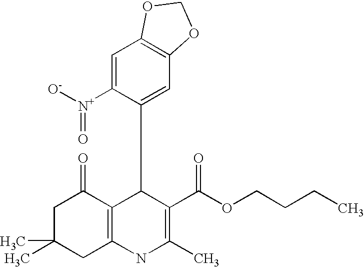 | 22.16999362 | 6.37350288 |
| ST216061 | pentyl 4-(3,4-dichlorophenyl)- 2,7,7-trimethyl-5-oxo-1,4,6,7,8- pentahydroquinoline-3-carboxylate | 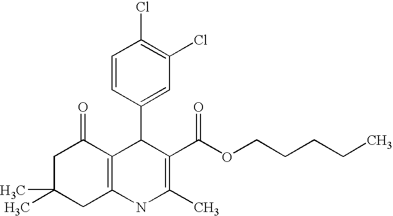 | 0 | 0 |
| ST216079 | pentyl 4-(2-bromophenyl)-2,7,7- trimethyl-5-oxo-1,4,6,7,8- pentahydroquinoline-3-carboxylate | 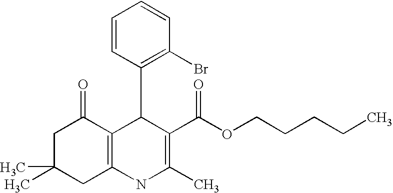 | 25.79796228 | 20.45245061 |
| ST216093 | pentyl 2,7,7-trimethyl-4-(4- nitrophenyl)-5-oxo-1,4,6,7,8- pentahydroquinoline-3-carboxylate | 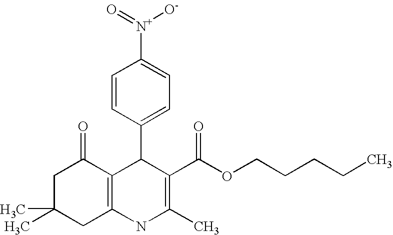 | 0 | 0 |
| ST216568 | pentyl 4-(6-bromo(2H-benzo[d]1,3- dioxolan-5-yl))-2,7,7-trimethyl-5- oxo-1,4,6,7,8- pentahydroquinoline-3-carboxylate | 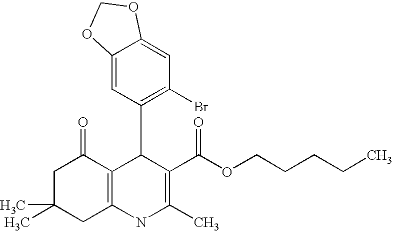 | 14.78651761 | 24.71178625 |
| ST216581 | pentyl 4-(2H-benzo[3,4-d]1,3- dioxolan-5-yl)-2,7,7-trimethyl-5- oxo-1,4,6,7,8- pentahydroquinoline- 3-carboxylate | 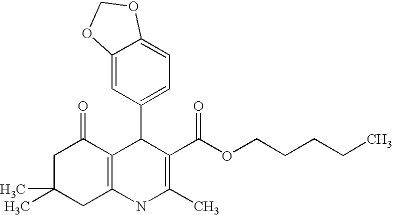 | 28.62736478 | 41.64007119 |
| ST216602 | pentyl 2,7,7-trimethyl-4-(4- methylphenyl)-5-oxo-1,4,6,7,8- pentahydroquinoline-3-carboxylate | 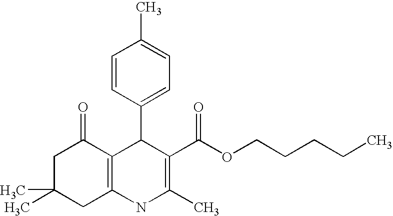 | 2.651500512 | 5.008345397 |
| ST216606 | pentyl 4-(4-methoxyphenyl)-2,7,7- trimethyl-5-oxo-1,4,6,7,8- pentahydroquinoline-3-carboxylate | 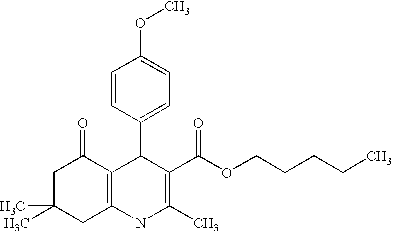 | 1.398215424 | 9.561084708 |
| ST216854 | phenylmethyl 4-(2-bromophenyl)- 2,7,7-trimethyl-5-oxo-1,4,6,7,8- pentahydroquinoline-3-carboxylate | 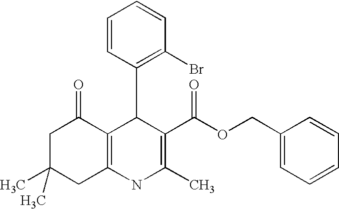 | 29.44984444 | 60.00769493 |
| ST216858 | phenylmethyl 4-(2-iodophenyl)- 2,7,7-trimethyl-5-oxo-1,4,6,7,8- pentahydroquinoline-3-carboxylate | 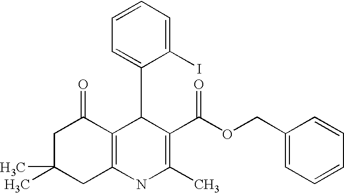 | 30.07449419 | 61.52564376 |
| ST216875 | methyl 2,7,7-trimethyl-5-oxo-4-(4- phenylphenyl)-1,4,6,7,8- pentahydroquinoline-3-carboxylate | 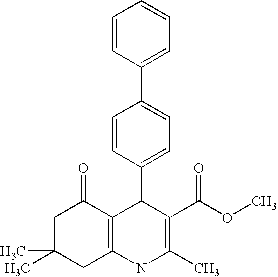 | 14.18179578 | 32.99019866 |
| ST216920 | cyclohexyl 4-(2H-benzo[3,4-d]1,3- dioxolen-5-yl)-2,7,7-trimethyl-5- oxo-1,4,6,7,8- pentahydroquinoline-3-carboxylate | 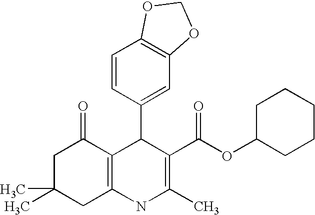 | 1.398215424 | 3.822551671 |
| ST217244 | pentyl 2,7,7-trimethyl-5-oxo-4-(4- propoxyphenyl)-1,4,6,7,8- pentahydroquinoline-3-carboxylate | 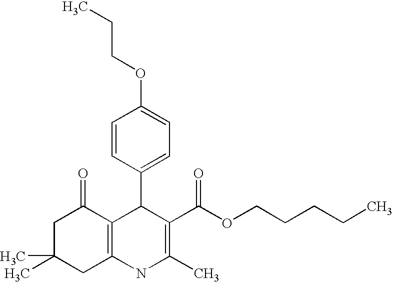 | 24.92584398 | 41.48949421 |
| ST024318 | 3,3-dimethyl-5-(2-methylphenyl)- 11-phenyl-2,3,4,5,11- pentahydroindeno[3,2-b]quinoline- 1,10-dione | 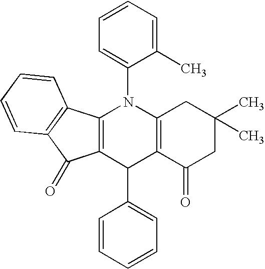 | 64.94486384 | 99.52632853 |

TABLE 2
Dihydropyridine Compounds

| | | | Aβ1-40 | Aβ1-42 |
|---|---|---|---|---|
| | | | (% of | (% of |
| ID | IUPAC Name | Structure | control) | control) |
| ST003359 | 5-amino-7-(2-methylphenyl)-2-[(2- methylphenyl)methylene]-3-oxo- 4,7-dihydro-1,3-thiazolidino[3,2- a]pyridine-6,8-dicarbonitrile | 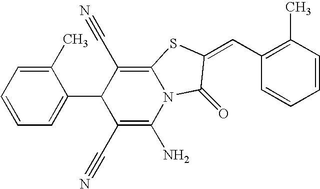 | 56.16135647 | 98.22334844 |
| ST003360 | 5-amino-7-(2,4-dichlorophenyl)-2- [(2,4-dichlorophenyl)methylene]-3- oxo-4,7-dihydro-1,3- thiazolidino[3,2-a]pyridine-6,8- dicarbonitrile | 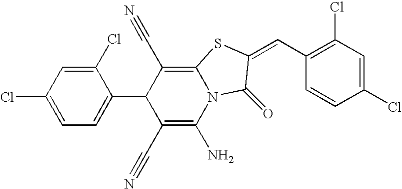 | 63.36955577 | 72.55108602 |
| ST003361 | 5-amino-7-(1-methylpyrrol-2-yl)-2- [(1-methylpyrrol-2-yl)methylene]- 3-oxo-4,7-dihydro-1,3- thiazolidino[3,2-a]pyridine-6,8- dicarbonitrile | 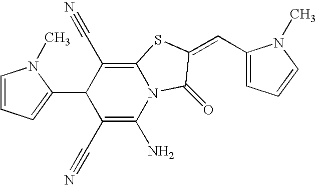 | 43.90448665 | 90.24380022 |
| ST003836 | ethyl 5-(ethoxycarbonyl)-2,6- dimethyl-4-[3-(2,2,2- trifluoroacetylamino)phenyl]-1,4- dihydropyridine-3-carboxylate | 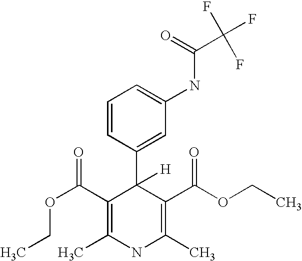 | 45.33858962 | 83.98396812 |
| ST006952 | 5-amino-7-(3-methyl(2-thienyl))-2- [(3-methyl(2-thienyl))methylene]- 3-oxo-4,7-dihydro-1,3- thiazolidino[3,2-a]pyridine-6,8- dicarbonitrile | 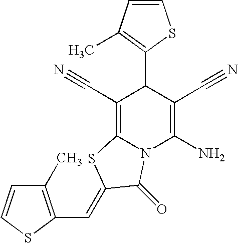 | 48.23610569 | 77.09937943 |
| ST006953 | 5-amino-7-(2-chlorophenyl)-2-[(2- chlorophenyl)methylene[-3-oxo- 4,7-dihydro-1,3-thiazolidino[3,2- a]pyridine-6,8-dicarbonitrile | 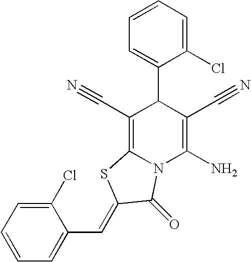 | 47.74097379 | 83.98396812 |
| ST007526 | (5-cyano-2-methyl-4-(4-pyridyl)-6- sulfanyl(3-1,4-dihydropyridyl))-N- benzamide, 4-methylmorpholine salt | 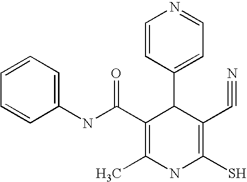 | 72.49756883 | 81.23471202 |
| ST011325 | ethyl 5-amino-7-(2- methoxyphenyl)-2-[(2- methoxyphenyl)methylene[-3-oxo- 8-(phenylsulfonyl)-4,7-dihydro- 1,3-thiazolidino[3,2-a]pyridine-6- carboxylate | 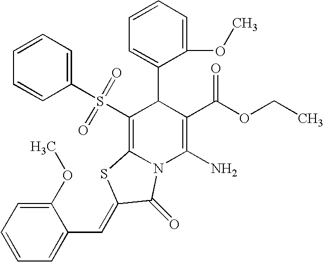 | 62.34579468 | 98.99938771 |
| ST011326 | ethyl 5-amino-7-[4- (methylethyl)phenyl]-2-{[4- methylethyl)phenyl[methylene}-3- oxo-8-(phenylsulfonyl)-4,7- dihydro-1,3-thiazolidino[3,2- a]pyridine-6-carboxylate | 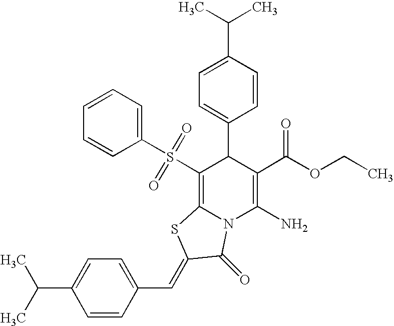 | 54.93556481 | 72.94687422 |
| ST011596 | prop-2-enyl 5-cyano-2-methyl-4-(3- pyridyl)-6-sulfanyl-1,4- dihydropyridine-3-carboxylate | 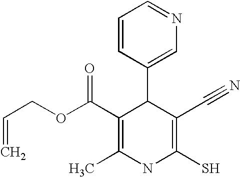 | 66.76848449 | 86.13954192 |
| ST011628 | 5-acetyl-6-methyl-4-(2- nitrophenyl)-2-sulfanyl-1,4- dihydropyridine-3-carbonitrile | 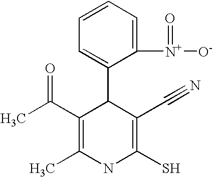 | 74.06670777 | 76.90148533 |
| ST011764 | ethyl 5-amino-6-(ethoxycarbonyl)- 7-[4-(methylethyl)phenyl]-2-{[4- (methylethyl) phenyl]methylene}- 3-oxo-4,7-dihydro-1,3- thiazolidino[3,2-a]pyridine-8- carboxylate | 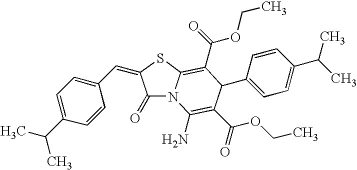 | 83.40407892 | 70.36852666 |
| ST011765 | ethyl 5-amino-6-(ethoxycarbonyl)- 7-(2-furyl)-2-(2-furylmethylene)-3- oxo-4,7-dihydro-1,3- thiazolidino[3,2-a]pyridine-8- carboxylate | 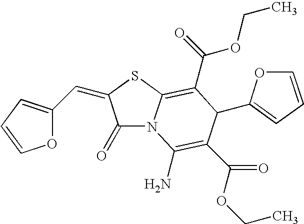 | 42.57087557 | 29.42161973 |
| ST011766 | ethyl 5-amino-6-cyano-7-(5- methyl(2-thienyl))-2-[(5-methyl(2- thienyl))methylene[-3-oxo-4,7- dihydro-1,3-thiazolidino[3,2- a]pyridine-8-carboxylate | 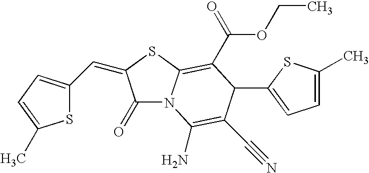 | 21.0970155 | 9.557794585 |
| ST011768 | methyl 5-amino-6- (methoxycarbonyl)-3-oxo-7-(3- pyridyl)-2-(3-pyridylmethylene)- 4,7-dihydro-1,3-thiazolidino[3,2- a]pyridine-8-carboxylate | 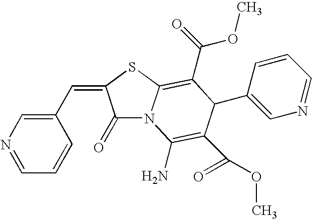 | 42.70591154 | 97.83491993 |
| ST011769 | methyl 5-amino-7-(2,4- dichlorophenyl)-2-[(2,4- dichlorophenyl)methylene]-6- (methoxycarbonyl)-3-oxo-4,7-dihydro- 1,3-thiazolidino[3,2-a]pyridine-8- carboxylate | 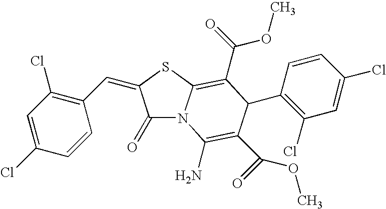 | 42.30080362 | 71.55916226 |
| ST014038 | methyl 2-(5-acetyl-3-cyano-6- methyl-4-(2-thienyl)-2-1,4- dihydropyridylthio)acetate | 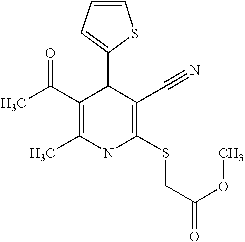 | 29.98208566 | 21.81866477 |
| ST014052 | methyl 4-(2H,3H-benzo[3,4-e]1,4- dioxan-6-yl)-5-cyano-2-methyl-6- (2-oxo-2-(2-thienyl)ethylthio)-1,4- dihydropyridine-3-carboxylate | 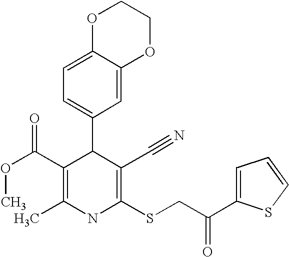 | 49.00114 | 74.68981241 |
| ST014065 | 6-amino-4-(3-bromophenyl)-2- sulfanyl-1,4-dihydropyridine-3,5- dicarbonitrile | 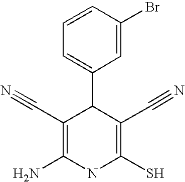 | 18.60105315 | 30.00509113 |
| ST014144 | 2-amino-4-(4-chlorophenyl)-5-oxo- 1,4-dihydrochromeno[4,3- b]pyridine-3-carbonitrile | 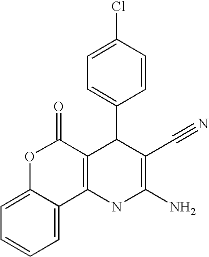 | 66.07676022 | 66.02213793 |
| ST014147 | 2-amino-4-(4-bromophenyl)-5-oxo- 1,4-dihydrochromeno[4,3- b]pyridine-3-carbonitrile | 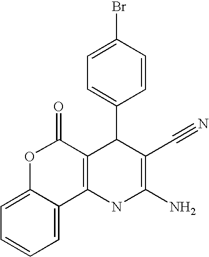 | 82.08702025 | 68.55337805 |
| ST014245 | 2-amino-1-benzyl-4-(3-pyridyl)-8- (3-pyridylmethylene)-1,4,7- trihydro-5H-pyrano[4,3- b]pyridine-3-carbonitrile | 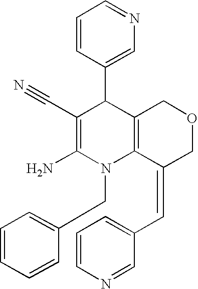 | 75.4356441 | 53.59354065 |
| ST024405 | ethyl 5-amino-8-cyano-3-oxo-7-(4- pyridyl)-2-(4-pyridylmethylene)- 4,7-dihydro-1,3-thiazolidino[3,2- a]pyridine-6-carboxylate | 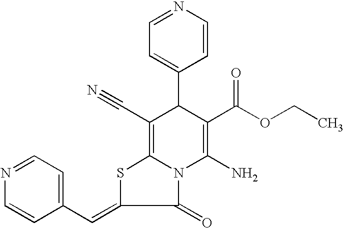 | 59.66259211 | 77.40725374 |
| ST024552 | methyl 5-(methoxycarbonyl)-1,2,6- trimethyl-4-(3-phenoxyphenyl)-1,4- dihydropyridine-3-carboxylate | 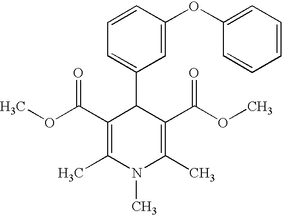 | 57.25769736 | 74.81070428 |
| ST024568 | methyl 5-(methoxycarbonyl)-1,2,6- trimethyl-4-(2-pyridyl)-1,4- dihydropyridine-3-carboxylate | 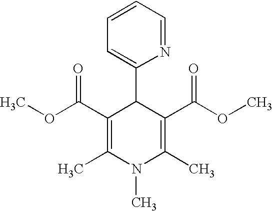 | 67.61180756 | 70.13103758 |
| ST024836 | methyl 4-(4-chlorophenyl)-1-[(4- fluorophenyl)methyl]-5- (methoxycarbonyl)-1,4- dihydropyridine-3-carboxylate | 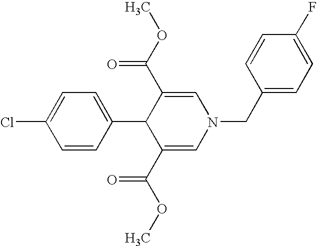 | 26.40699115 | 82.553677 |
| ST024841 | 5-amino-7-(3-bromophenyl)-2-[(3- bromophenyl)methylene]-3-oxo- 4,7-dihydro-1,3-thiazolidino[3,2- a]pyridine-6,8-dicarbonitrile | 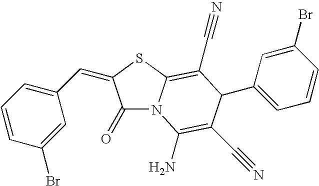 | 61.77605667 | 95.58309996 |
| ST025017 | 5-amino-7-(2,4-dimethoxyphenyl)- 2-[(2,4- dimethoxyphenyl)methylene]-3- oxo-4,7-dihydro-1,3- thiazolidino[3,2-a]pyridine-6,8- dicarbonitrile | 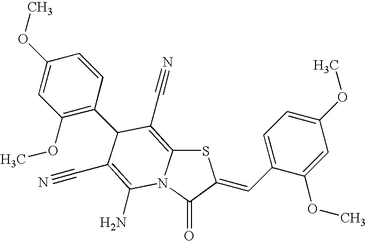 | 64.5355116 | 72.3852989 |
| ST025022 | 5-amino-7-(4-ethoxy-3- methoxyphenyl)-2-[(4-ethoxy-3- methoxyphenyl)methylene]-3-oxo- 4,7-dihydro-1,3-thiazolidino[3,2- a]pyridine-6,8-dicarbonitrile | 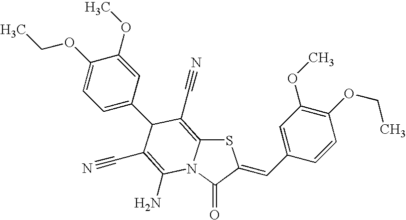 | 61.31389759 | 76.11070774 |
| ST025023 | 5-amino-7-[3-(2- methylpropoxy)phenyl]-2-{[3-(2- methylpropoxy)phenyl]methylene}- 3-oxo-4,7-dihydro-1,3- thiazolidino[3,2-a]pyridine-6,8- dicarbonitrile | 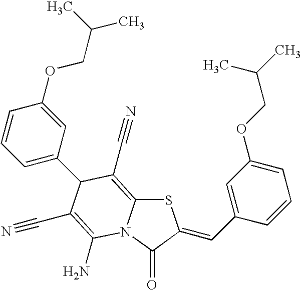 | 68.12915573 | 85.2885247 |
| ST025026 | 5-amino-7-[4- (methylethoxy)phenyl]-2-{[4- (methylethoxy)phenyl]methylene}- 3-oxo-4,7-dihydro-1,3- thiazolidino[3,2-a]pyridine-6,8- dicarbonitrile | 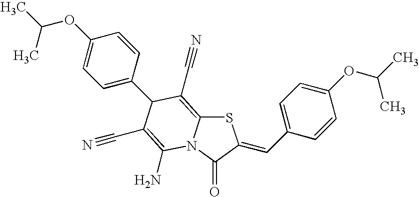 | 26.82031382 | 0 |
| ST025029 | 5-amino-7-[3-methoxy-4- (methylethoxy)phenyl]-2-{[3- methoxy-4-(methylethoxy)phenyl] methylene}-3-oxo-4,7-dihydro- 1,3-thiazolidino[3,2-a]pyridine-6,8- dicarbonitrile | 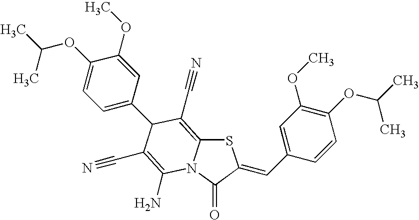 | 65.70242356 | 73.69394599 |
| ST025030 | 5-amino-7-(5-bromo-2- hydroxyphenyl)-2-[(5-bromo-2- hydroxyphenyl)methylene]-3-oxo- 4,7-dihydro-1,3-thiazolidino[3,2- a]pyridine-6,8-dicarbonitrile | 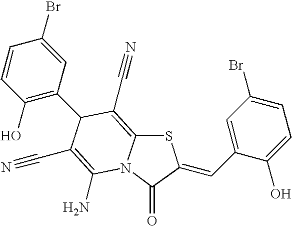 | 39.28510927 | 33.92801577 |
| ST025031 | 5-amino-3-oxo-7-(3,4,5- trimethoxyphenyl)-2-[(3,4,5- trimethoxyphenyl)methylene]-4,7- dihydro-1,3-thiazolidino[3,2- a]pyridine-6,8-dicarbonitrile | 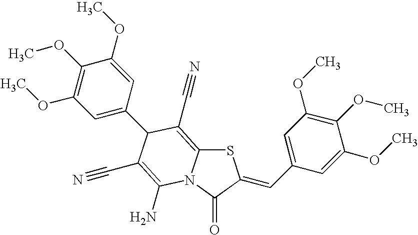 | 55.16845248 | 77.59222764 |
| ST025032 | 5-amino-7-(4-hydroxy-3- methoxyphenyl)-2-[(4-hydroxy-3- methoxyphenyl)methylene]-3-oxo- 4,7-dihydro-1,3-thiazolidino[3,2- a]pyridine-6,8-dicarbonitrile | 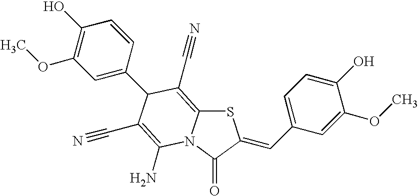 | 27.77083686 | 32.98067282 |
| ST025033 | 5-amino-7-(3-ethoxy-4- hydroxyphenyl)-2-[(3-ethoxy-4- hydroxyphenyl)methylene]-3-oxo- 4,7-dihydro-1,3-thiazolidino[3,2- a]pyridine-6,8-dicarbonitrile | 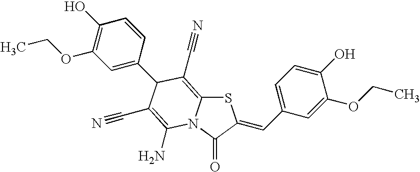 | 53.66167097 | 57.59775957 |
| ST025034 | 5-amino-7-(4-hydroxyphenyl)-2- [(4-hydroxyphenyl)methylene]-3- oxo-4,7-dihydro-1,3- thiazolidino[3,2-a]pyridine-6,8- dicarbonitrile | 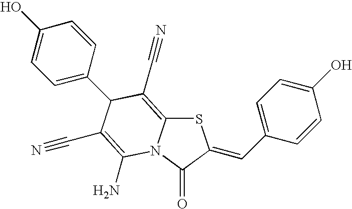 | 69.9007655 | 85.10700826 |
| ST025037 | 2-[4-(5-amino-2-{[4- (carbamoylmethoxy)-3- ethoxyphenyl]methylene}-6,8- dicyano-3-oxo(4,7-dihydro-1,3- thiazolidino[3,2-a]pyridin-7-yl))-2- ethoxyphenoxy]acetamide | 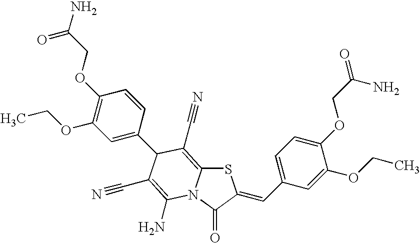 | 80.02022277 | 67.09884867 |
| ST025089 | ethyl 5-amino-6-cyano-3-oxo-7-[2- (trifluoromethyl)phenyl]-2-{[2- (trifluoromethyl)phenyl]methylene}- 4,7-dihydro- 1,3-thiazolidino[3,2-a]pyridine-8- carboxylate | 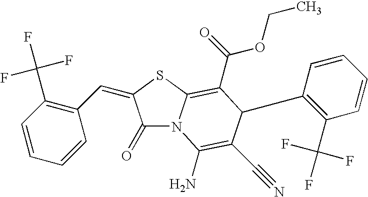 | 48.7065634 | 33.69290876 |
| ST025482 | 6-amino-2-(dicyanomethyl)-4-(3,4- dimethoxyphenyl)-1,4- dihydropyridine-3,5-dicarbonitrile | 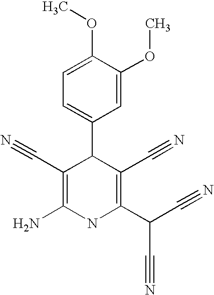 | 62.82901703 | 45.10078484 |
| ST026470 | ethyl 1-[4-(acetylamino)(1,2,5- oxadiazol-3-yl)]-6-amino-4-(2,4- dichlorophenyl)-5-cyano-2-methyl- 1,4-dihydropyridine-3-carboxylate | 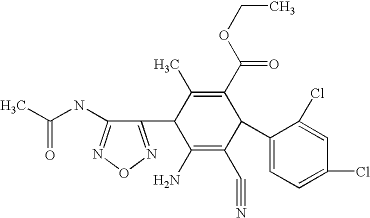 | 81.69535088 | 67.47916883 |
| ST026471 | ethyl 1-[4-(acetylamino)(1,2,5- oxadiazol-3-yl)]-6-amino-4-(2- chlorophenyl)-5-cyano-2-methyl- 1,4-dihydropyridine-3-carboxylate | 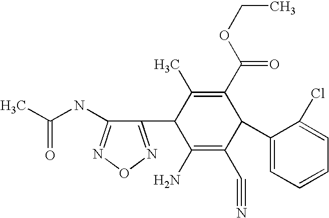 | 4.367482636 | 0 |
| ST038081 | methyl 4-(2,4-dimethoxyphenyl)-1- [(4-fluorophenyl)methyl]-5- (methoxycarbonyl)-1,4- dihydropyridine-3-carboxylate | 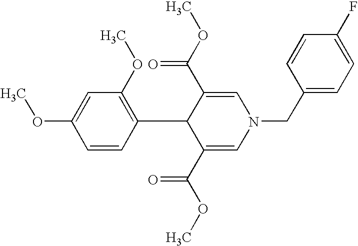 | 36.22274105 | 92.93110807 |
| ST038110 | methyl 1-(2H-benzo[3,4-d]1,3- dioxolen-5-ylmethyl)-4-(2,3- dimethoxyphenyl)-5- (methoxycarbonyl)-1,4- dihydropyridine-3-carboxylate | 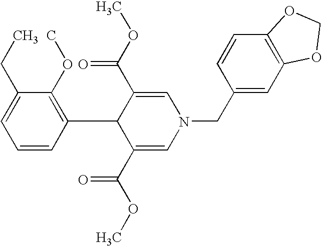 | 39.35067209 | 74.78258485 |
| ST038112 | methyl 1-(2H-benzo[3,4-d]1,3- dioxolen-5-ylmethyl)-5- (methoxycarbonyl)-4-(2-thienyl)- 1,4-dihydropyridine-3-carboxylate | 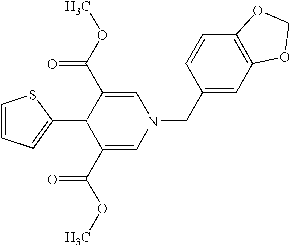 | 47.6979228 | 99.51994629 |
| ST038114 | methyl 1-(2H-benzo[3,4-d]1,3- dioxolen-5-ylmethyl)-4-(3,4- dimethoxyphenyl)-5- (methoxycarbonyl)-1,4- dihydropyridine-3-carboxylate | 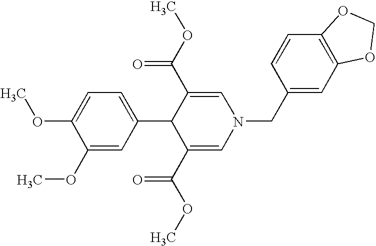 | 48.00059392 | 95.91618031 |
| ST038115 | methyl 1-[(3,4- dimethoxyphenyl)methyl]-5- (methoxycarbonyl)-4-(2-thienyl)- 1,4-d ihydropyridine-3-carboxylate | 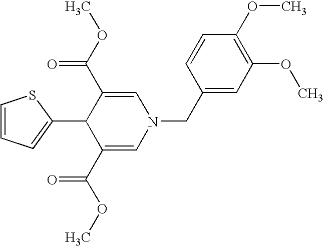 | 42.44553189 | 90.99691958 |
| ST038162 | methyl 4-(3,4-dimethoxyphenyl)-1- [(3,4-dimethoxyphenyl)methyl]-5- (methoxycarbo nyl)-1,4- dihydropyridine-3-carboxylate | 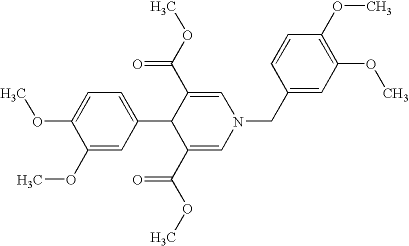 | 49.64720096 | 58.6241508 |
| ST038735 | methyl 1-cyclopropyl-4-(3- fluorophenyl)-5- (methoxycarbonyl)-1,4- dihydropyridine-3-carboxylate | 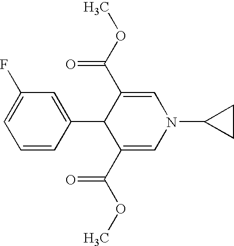 | 65.36287539 | 98.51948067 |
| ST038738 | methyl 4-(4-chlorophenyl)-1- cyclopropyl-5-(methoxycarbonyl)- 1,4-dihydropyridine-3-carboxylate | 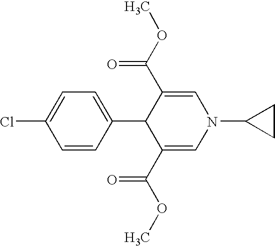 | 94.48516721 | 52.58896601 |
| ST041706 | N-(3,4-dimethylphenyl)-2-[3- cyano-6-methyl-5-(N- phenylcarbamoyl)-4-(2-thienyl) (2- 1,4-dihydropyridylthio)]acetamide | 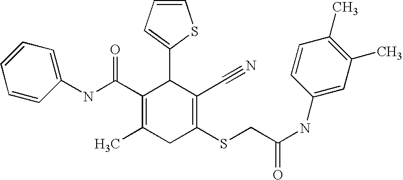 | 68.73375602 | 50.1272459 |
| ST045237 | methyl 4-(3,4-dichlorophenyl)-5- (methoxycarbonyl)-1-(oxolan-2- ylmethyl)-1,4-dihydropyridine-3- carboxylate | 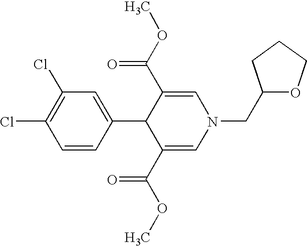 | 60.45503462 | 50.7708875 |
| ST045329 | methyl 1-adamantanyl-5- (methoxycarbonyl)-4-(3- nitrophenyl)-1,4-dihydropyridine- 3-carboxylate | 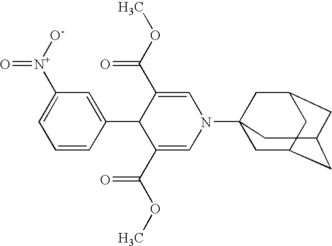 | 82.59951979 | 68.51596076 |
| ST045410 | methyl 4-[4-(2,4-dichlorophenyl)(2- furyl)]-5-(methoxycarbonyl)-2,6- dimethyl-1,4-dihydropyridine-3- carboxylate | 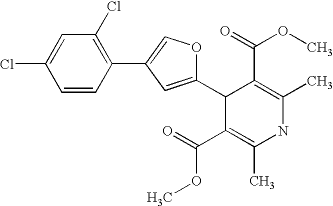 | 75.12601779 | 69.44433016 |
| ST048506 | 2-[5-acetyl-3-cyano-6-methyl-4-(4- methyl(2-thienyl))(2-1,4- dihydropyridylthio)]-N-(2,4- dibromo-6-methylphenyl)acetamide | 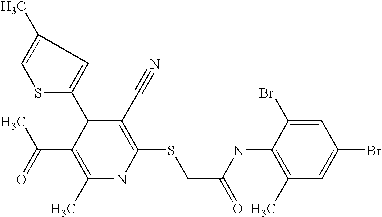 | 91.15791889 | 65.5758358 |
| ST050424 | tert-butyl 6-{[N-(2,3- dimethylphenyl)carbamoyl]methylthio}- 4-(4-chlorophenyl)-5-cyano- 2-methyl-1,4-dihydropyridine-3- carboxylate | 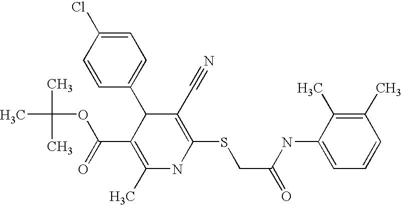 | 41.20893606 | 52.28527934 |
| ST050428 | tert-butyl 6-{[N-(2,5- dimethylphenyl)carbamoyl]methylthio}- 4-(4-chlorophenyl)-5-cyano- 2-methyl-1,4-dihydropyridine-3- carboxylate | 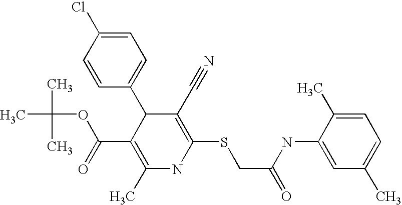 | 95.73847964 | 65.46912006 |
| ST050837 | 2-[3-cyano-6-methyl-5-(N- phenylcarbamoyl)-4-(2-thienyl)(2- 1,4-dihydropyridylthio)]-N-(2- methylphenyl)acetamide | 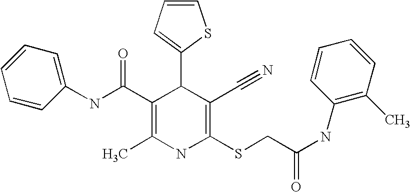 | 95.44262887 | 41.28911135 |
| ST050838 | 2-{3-cyano-6-methyl-5-[N-(2- methylphenyl)carbamoyl]-4-(2- thienyl)(2-1,4-dihydropyridylthio)}- N-(4-ethoxyphenyl)acetamide | 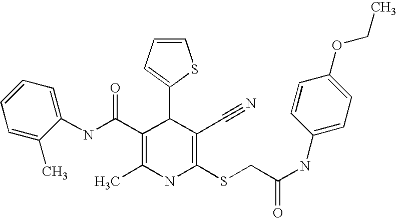 | 70.7588621 | 56.65716641 |
| ST050840 | N-(3-chloro-4-methylphenyl)-2-{3- cyano-6-methyl-5-[N-(2- methylphenyl)carbamoyl]-4-(2- thienyl)(2-1,4- dihydropyridylthio)}acetamide | 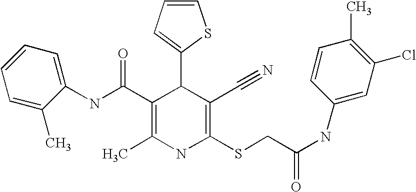 | 52.30260749 | 38.84354225 |
| ST050842 | N-(3-chloro-4-methylphenyl)-2-[3- cyano-6-methyl-5-(N- phenylcarbamoyl)-4-(2-thienyl)(2- 1,4-dihydropyridylthio)]acetamide | 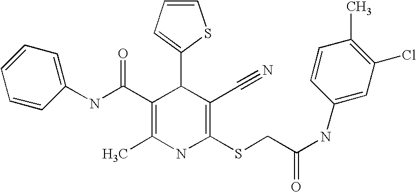 | 37.46466634 | 52.05801433 |
| ST050859 | 2-[6-amino-4-(2-chlorophenyl)-3,5- dicyano-2-1,4- dihydropyridylthio]acetamide | 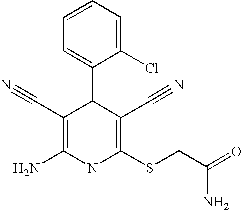 | 75.54080641 | 35.46766877 |
| ST050862 | methyl 2-{5-[(tert- butyl)oxycarbonyl]-4-(4- chlorophenyl)-3-cyano-6-methyl-2- 1,4-dihydropyridylthio}acetate | 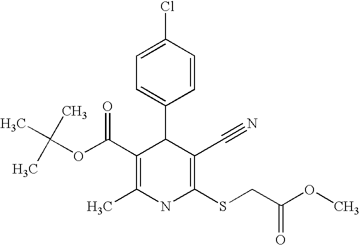 | 65.24095239 | 56.21301154 |
| ST050983 | tert-butyl 4-(4-chlorophenyl)-5- cyano-2-methyl-6-methylthio-1,4- dihydropyridine-3-carboxylate | 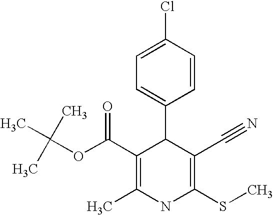 | 51.29788657 | 0 |
| ST051142 | tert-butyl 4-(4-chlorophenyl)-5- cyano-2-methyl-6-{[N-(4- methylphenyl)carbamoyl]methylthio}- 1,4-dihydropyridine- 3-carboxylate | 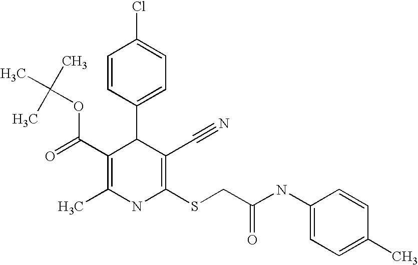 | 94.84873043 | 46.05871219 |
| ST051153 | 2-{5-[N-(4- chlorophenyl)carbamoyl]-3-cyano- 6-methyl-4-phenyl(2-1,4- dihydropyridylthio)}-N-(3- methylphenyl)acetamide | 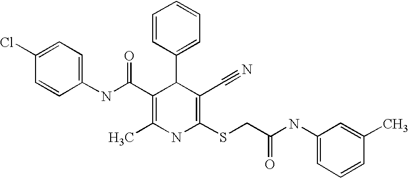 | 4.932798914 | 0 |
| ST051156 | N-(3-chloro-2-methylphenyl)-2-{3- cyano-4-(2-furyl)-6-methyl-5-[N- (2-methylphenyl)carbamoyl](2- 1,4-dihydropyridylthio)}acetamide | 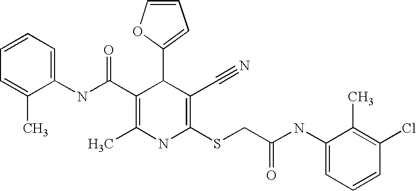 | 86.63960397 | 68.02140491 |
| ST051158 | 2-{3-cyano-4-(2-furyl)-6-methyl-5- [N-(2-methylphenyl)carbamoyl](2- 1,4-dihydropyridylthio)}-N-(4- ethoxyphenyl)acetamide | 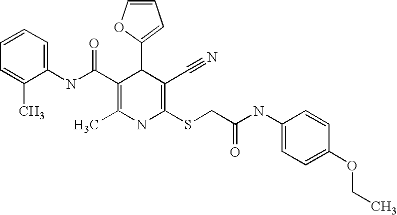 | 17.68147711 | 15.86902619 |
| ST051458 | methyl 4-(6-chloro(2H- benzo[d]1,3-dioxolan-5-yl))-1- cyclopropyl-5-(methoxycarbonyl)- 1,4-dihydropyridine-3-carboxylate | 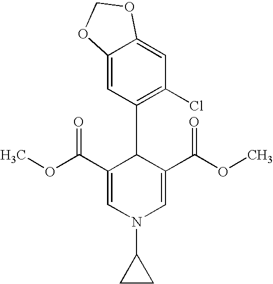 | 77.80704152 | 59.89749054 |
| ST051459 | methyl 4-(2,3-dimethoxyphenyl)-5- (methoxycarbonyl)-1-(oxolan-2- ylmethyl)-1,4-dihydropyridine-3- carboxylate | 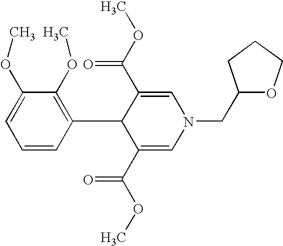 | 70.09383722 | 85.95722149 |
| ST051569 | methyl 4-(3-bromophenyl)-5- (methoxycarbonyl)-1-(oxolan-2- ylmethyl)-1,4-dihydropyridine-3- carboxylate | 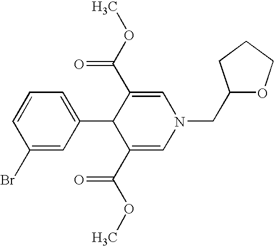 | 49.02435792 | 72.76818319 |
| ST052261 | methyl 5-(methoxycarbonyl)-4-(3- methoxyphenyl)-1,4- dihydropyridine-3-carboxylate | 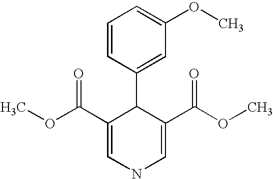 | 38.04473794 | 57.23929616 |
| ST052273 | methyl 5-(methoxycarbonyl)-4-[3- (phenylmethoxy)phenyl]-1,4- dihydropyridine-3 -carboxylate | 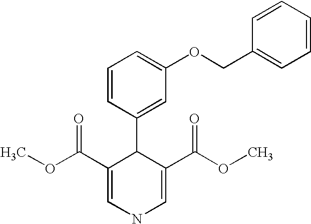 | 84.65948538 | 57.9217965 |
| ST052274 | methyl 4-(4-ethylphenyl)-5- (methoxycarbonyl)-1,4- dihydropyridine-3-carboxylate | 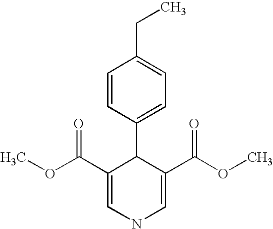 | 4.951507642 | 0 |
| ST052284 | ethyl 5-(ethoxycarbonyl)-4-(2- ethoxyphenyl)-1-(4-methylphenyl)- 1,4-dihydropyridine-3-carboxylate | 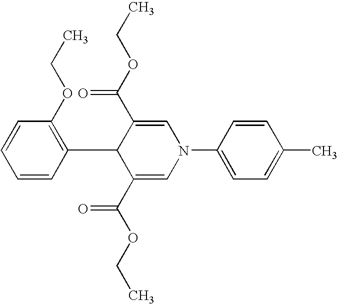 | 57.7133824 | 55.60852182 |
| ST052316 | ethyl 5-(ethoxycarbonyl)-4-(2- ethoxyphenyl)-1-(4-fluorophenyl)- 1,4-dihydropyridine-3-carboxylate | 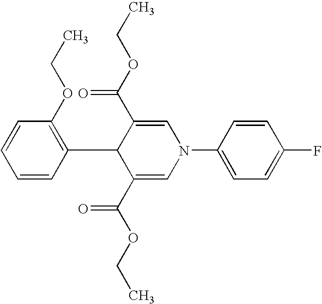 | 68.08983904 | 40.0250317 |
| ST052327 | ethyl 5-(ethoxycarbonyl)-1-(4- fluorophenyl)-4-(2-thienyl)-1,4- dihydropyridine-3-carboxylate | 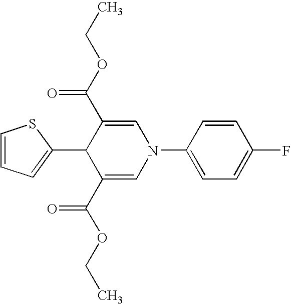 | 57.73821604 | 84.32974201 |
| ST052366 | ethyl 5-(ethoxycarbonyl)-1,2,6- trimethyl-4-(2-pyridyl)-1,4- dihydropyridine-3-carboxylate | 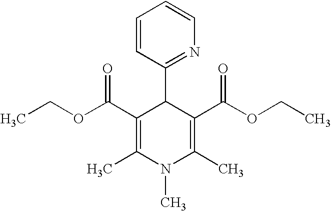 | 56.21321027 | 89.75762184 |
| ST052391 | methyl 4-(2,3-dimethoxyphenyl)-5- (methoxycarbonyl)-1- (phenylethyl)-1,4-dihydropyridine- 3-carboxylate | 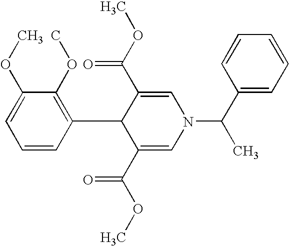 | 29.94609178 | 16.18693826 |
| ST052415 | methyl 4-(3-ethoxyphenyl)-5- (methoxycarbonyl)-1,4- dihydropyridine-3-carboxylate | 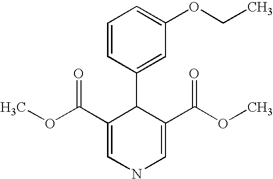 | 70.57396989 | 72.15123599 |
| ST052438 | methyl 4-(3-chlorophenyl)-5- (methoxycarbonyl)-1- (phenylethyl)-1,4-dihydropyridine- 3-carboxylate | 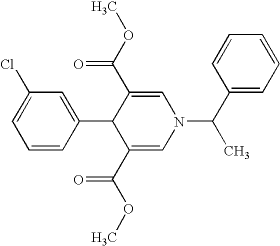 | 47.39643815 | 65.74128324 |
| ST052440 | methyl 4-(2-chlorophenyl)-5- (methoxycarbonyl)-1- (phenylethyl)-1,4-dihydropyridine- 3-carboxylate | 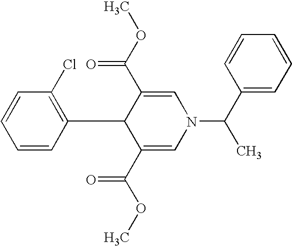 | 37.28977295 | 79.81317809 |
| ST052450 | ethyl 4-(2,3-dimethoxyphenyl)-1- (2,5-dimethylphenyl)-5- (ethoxycarbonyl)-1,4- dihydropyridine-3-carboxylate | 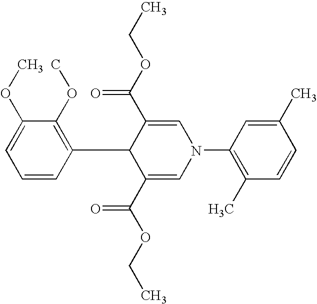 | 5.248627712 | 12.04080917 |
| ST052464 | methyl 4-(4-chlorophenyl)-5- (methoxycarbonyl)-1- (phenylethyl)-1,4-dihydropyridine- 3-carboxylate | 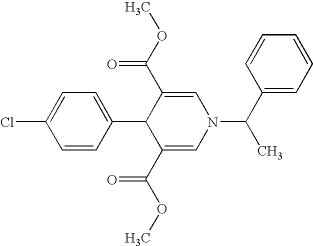 | 45.08487301 | 77.31121101 |
| ST052471 | ethyl 1-(2,5-dimethylphenyl)-5- (ethoxycarbonyl)-4-(4-ethoxy-3- methoxyphenyl)-1,4- dihydropyridine-3-carboxylate | 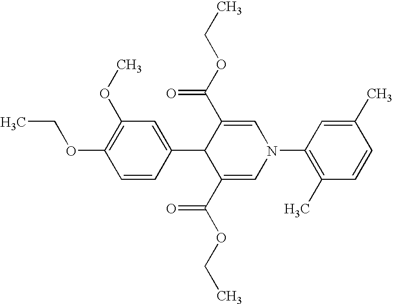 | 34.26430958 | 57.75436544 |
| ST052474 | ethyl 1-(2,5-dimethylphenyl)-5- (ethoxycarbonyl)-4-(2,4,5- trimethoxyphenyl)-1,4- dihydropyridine-3-carboxylate | 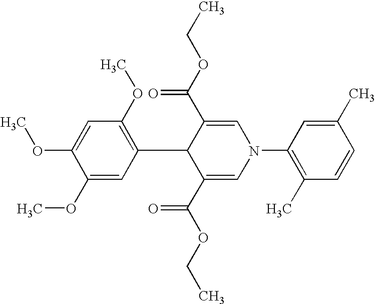 | 5.403755892 | 3.498108487 |
| ST052487 | ethyl 1-(2,5-dimethylphenyl)-5- (ethoxycarbonyl)-4-(2,3,4- trimethoxyphenyl)-1,4- dihydropyridine-3-carboxylate | 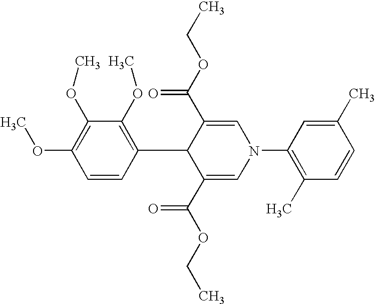 | 11.56301585 | 15.09861973 |
| ST052488 | ethyl 1-(2,5-dimethylphenyl)-5- (ethoxycarbonyl)-4-(2- ethoxyphenyl)-1,4- dihydropyridine-3-carboxylate | 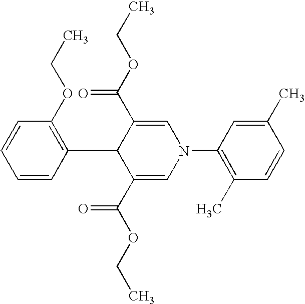 | 60.30421531 | 57.75436544 |
| ST052491 | ethyl 1-(2,5-dimethylphenyl)-5- (ethoxycarbonyl)-4-(3,4,5- trimethoxyphenyl)-1,4- dihydropyridine-3-carboxylate | 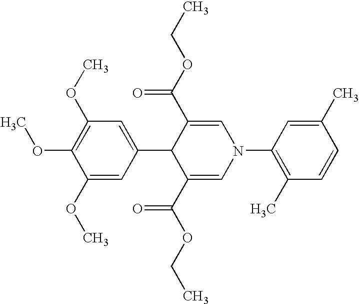 | 70.28611702 | 28.34522581 |
| ST063245 | ethyl 4-(4-bromophenyl)-5- (methoxycarbonyl)-2,6-dimethyl- 1,4-dihydropyridine-3-carboxylate | 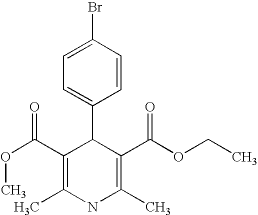 | 8.423161831 | 0 |
| ST208633 | methyl 4-(4-bromophenyl)-5- (methoxycarbonyl)-2,6-dimethyl- 1,4-dihydropyridine-3-carboxylate | 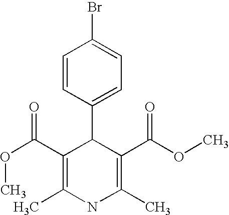 | 9.119741444 | 0 |
| ST208634 | methyl 4-(3-bromophenyl)-5- (methoxycarbonyl)-2,6-dimethyl- 1,4-dihydropyridine-3-carboxylate | 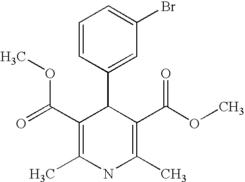 | 80.50155559 | 32.51078812 |
| ST211423 | ethyl 4-(2,6-dichlorophenyl)-5- (ethoxycarbonyl)-2,6-dimethyl-1,4- dihydropyridine-3-carboxylate | 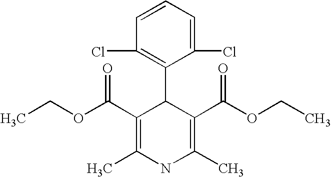 | 2.731212197 | 0 |
| ST211424 | methyl 4-(3,5-dichloro-2- methoxyphenyl)-5- (ethoxycarbonyl)-2,6-dimethyl-1,4- dihydropyridine-3-carboxylate | 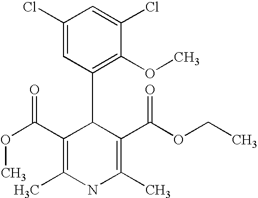 | 62.45627935 | 91.28508042 |
| ST211500 | ethyl 5-(ethoxycarbonyl)-4-(4- fluorophenyl)-2,6-dimethyl-1,4- dihydropyridine-3-carboxylate | 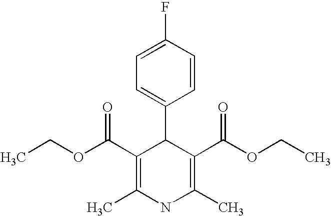 | 20.66923999 | 19.53293456 |
| ST211853 | 5-amino-3-oxo-7-(4-pyridyl)-2-(4- pyridylmethylene)-4,7-dihydro-1,3- thiazolidino[3,2-a]pyridine-6,8- dicarbonitrile | 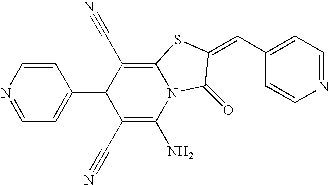 | 49.00185955 | 18.36946417 |
| ST215651 | ethyl 5-amino-7-(2-chlorophenyl)- 2-[(2-chlorophenyl)methylene]-8- cyano-3-oxo-4,7-dihydro-1,3- thiazolidino[3,2-a]pyridine-6- carboxylate | 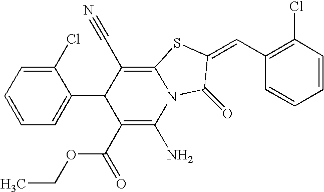 | 80.91612637 | 42.29873608 |
| ST215709 | 5-amino-7-(4-methylphenyl)-2-[(4- methylphenyl)methylene]-3-oxo- 4,7-dihydro-1,3-thiazolidino[3,2- a]pyridine-6,8-dicarbonitrile | 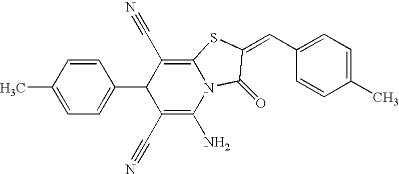 | 55.90767387 | 78.42869721 |
| ST215713 | 5-amino-7-(3-methoxyphenyl)-2- [(3-methoxyphenyl)methylene]-3- oxo-4,7-dihydro-1,3- thiazolidino[3,2-a]pyridine-6,8- dicarbonitrile | 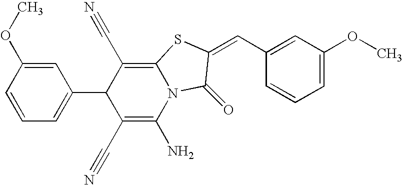 | 69.19835524 | 80.48369993 |
| ST217080 | [4-(2,6-dichlorophenyl)-2,6- dimethyl-5-(N- phenylcarbamoyl)(3-1,4- dihydropyridyl)]-N-benzamide | 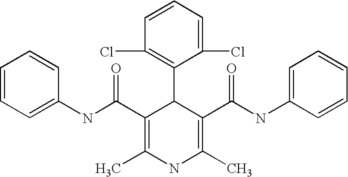 | 65.54257087 | 0 |

| ID | Structure | Aβ1-40 (% of control) | Aβ1-42 (% of control) |
|---|---|---|---|
| RI_1 | 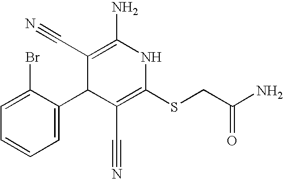 | 93.2 | 75.4 |
| RI_2 | 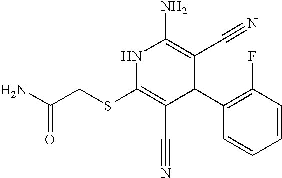 | 100.9 | 44.4 |
| RI_3 | 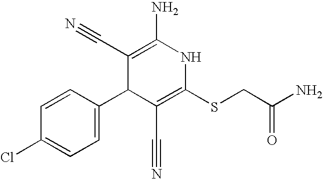 | 97.4 | 51.2 |
| RI_4 | 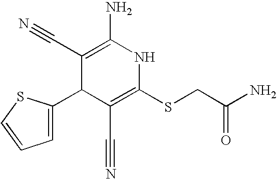 | 92.2 | 64.7 |
| RI_5 | 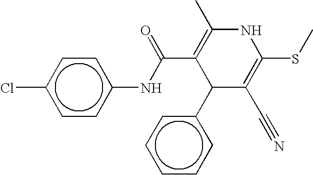 | 81.4 | 82.2 |
| RI_6 | 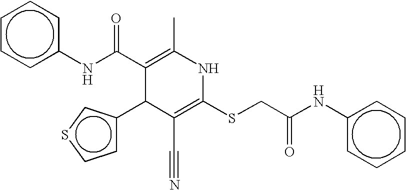 | 77.7 | 65.1 |
| RI_7 | 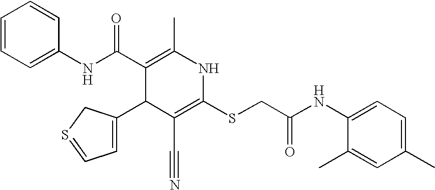 | 60.1 | 58.8 |
| RI_8 | 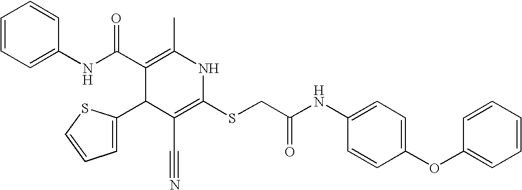 | 46.1 | 26.4 |
| RI_9 | 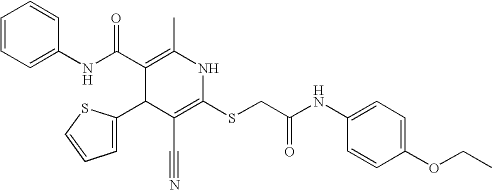 | 56.3 | 27.6 |
| RI_10 | 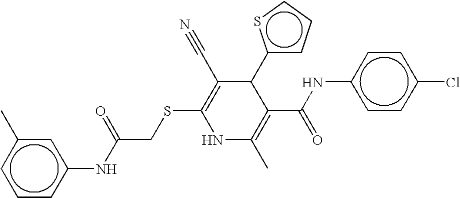 | 78.8 | 70.6 |
| RI_11 | 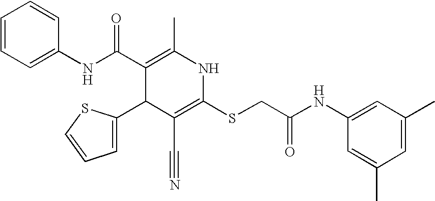 | 52.1 | 35.8 |
| RI_12 | 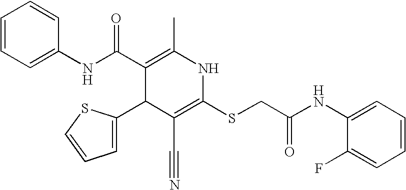 | 68.4 | 82.5 |
| RI_13 | 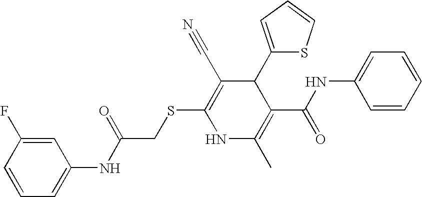 | 53.8 | 41.3 |
| RI_14 | 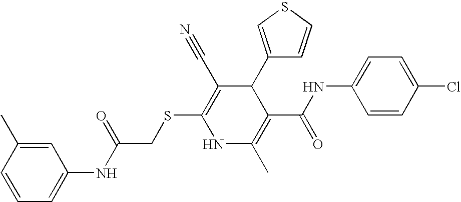 | 56.3 | 64.1 |
| RI_15 | 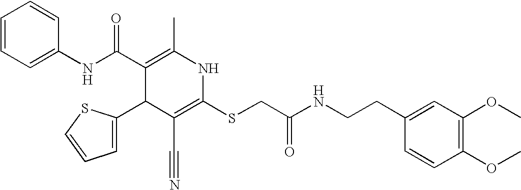 | 74.9 | 41.2 |
| RI_16 | 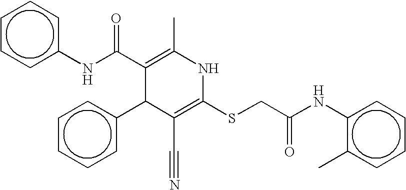 | 64.2 | 105 |
| RI_17 | 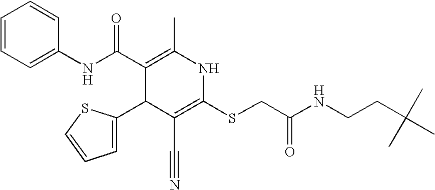 | 18.2 | 24.1 |
| RI_18 | 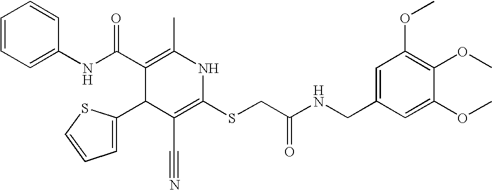 | 49.2 | 44.1 |
| RI_19 | 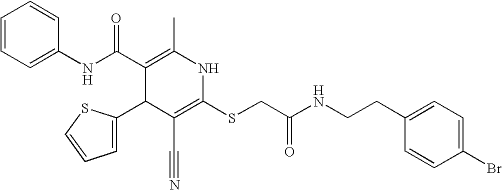 | 35.4 | 44.4 |
| RI_20 | 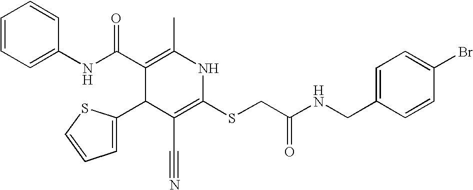 | 45.6 | 41.9 |
| RI_21 | 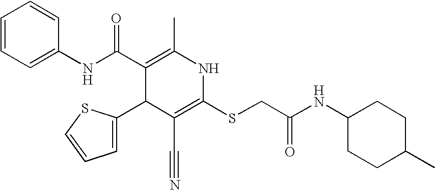 | 18.7 | 22.1 |
| RI_22 | 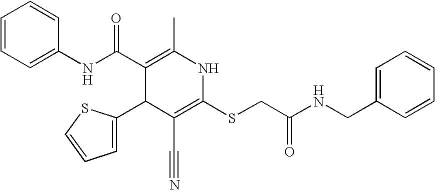 | 60.9 | 47.6 |
| RI_23 | 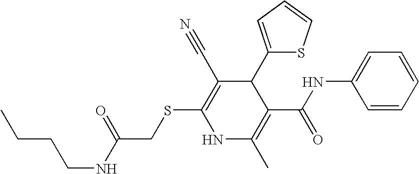 | 49.3 | 35.9 |
| RI_24 | 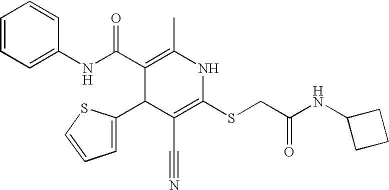 | 80.8 | 44.1 |
| RI_25 | 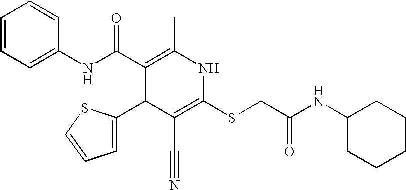 | 55.9 | 36.5 |
| RI_26 | 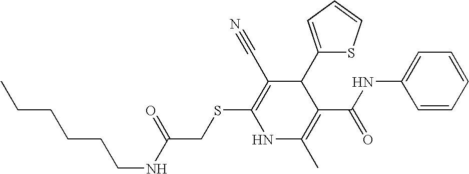 | 37.6 | 26.7 |
| RI_27 | 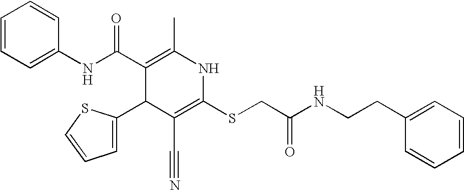 | 34.4 | 26.5 |
| RI_28 | 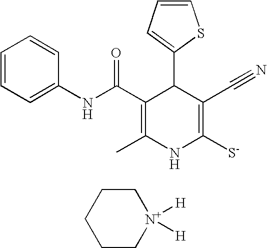 | 77.1 | 68.5 |
| RI_29 | 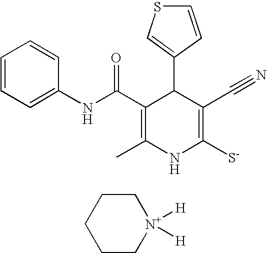 | 79.7 | 100 |
| RI_30 | 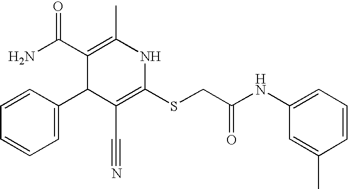 | 94.9 | 48.2 |
| RI_31 | 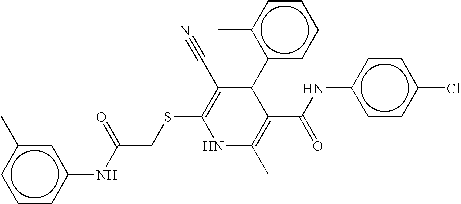 | 53.9 | 73.3 |
| RI_32 | 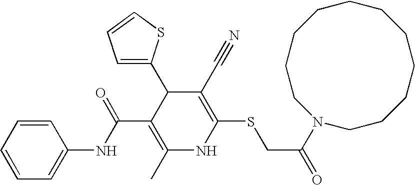 | 62.9 | 62.8 |
| RI_33 | 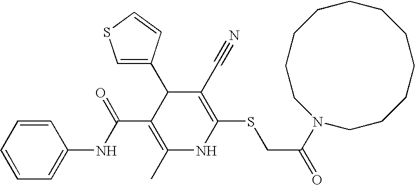 | 11.9 | 13.7 |
| RI_34 | 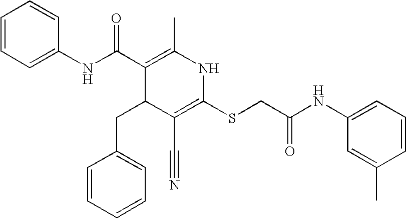 | 31.4 | 26.1 |
| RI_35 | 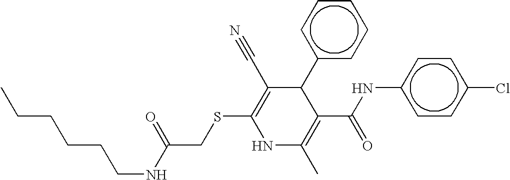 | 55.4 | 78.6 |
| RI_36 | 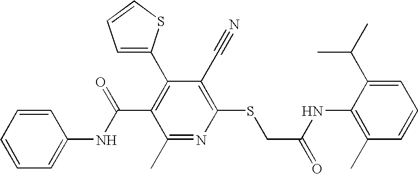 | 35.9 | 72.9 |
| RI_37 | 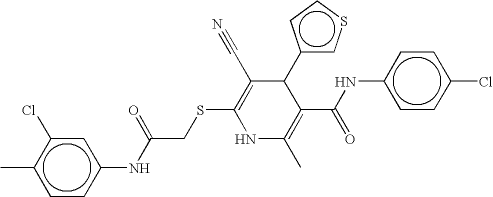 | 64.4 | 75.8 |
| RI_38 | 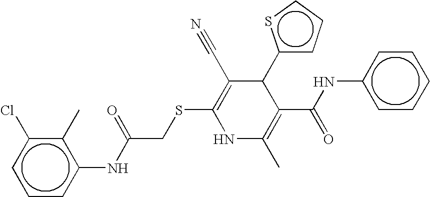 | 62.5 | 103.4 |
| RI_39 | 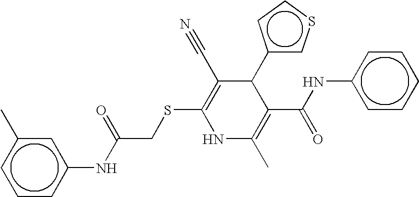 | 72 | 72 |
| RI_40 | 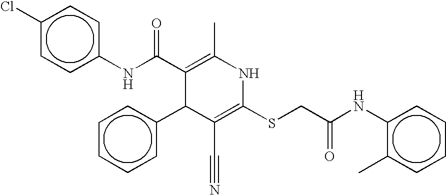 | 25.7 | 43.2 |
| RI_41 | 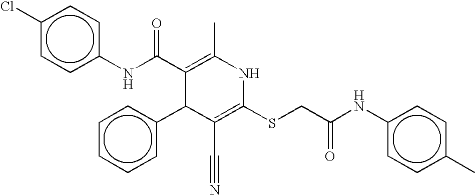 | 36.4 | 30.7 |
| RI_42 | 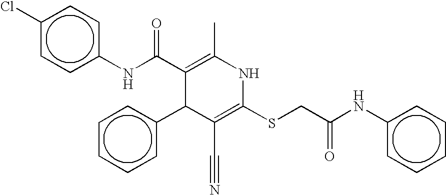 | 27.2 | 36.2 |
| RI_43 | 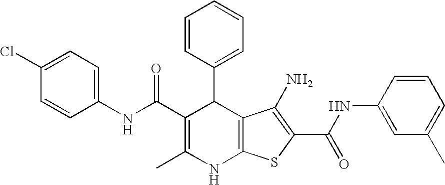 | 61.2 | 75.1 |
| RI_44 | 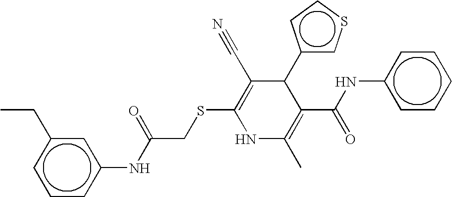 | 93.1 | 60.1 |
| RI_45 | 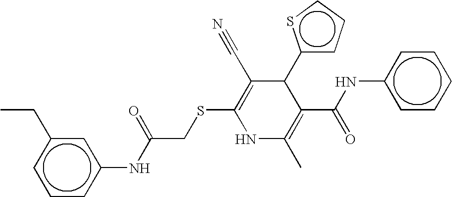 | 82.7 | 61.3 |
| RI_46 | 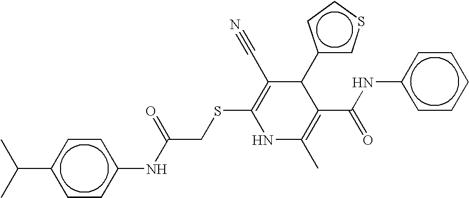 | 74.4 | 70.2 |
| RI_47 | 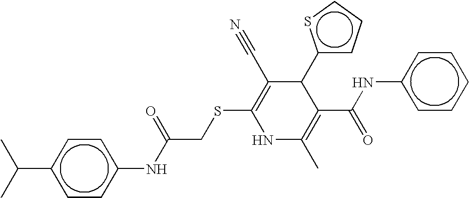 | 30.5 | 28.1 |
| RI_48 | 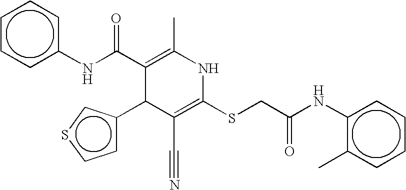 | 86.5 | 100 |
| RI_49 | 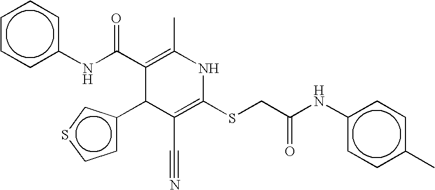 | 40.9 | 37.7 |
| RI_50 | 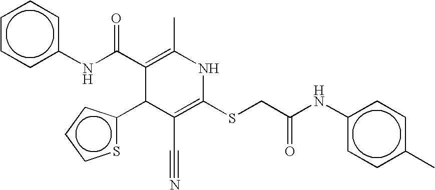 | 55.2 | 38.2 |
| RI_51 | 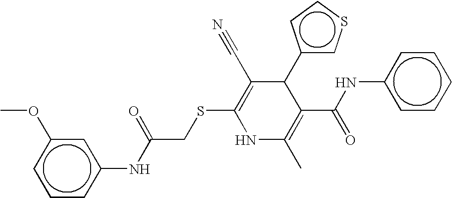 | 53.5 | 58 |
| RI_52 | 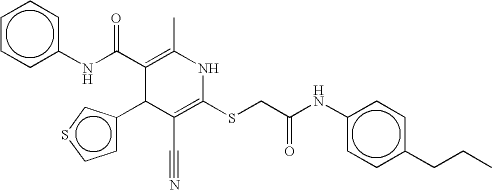 | 50.3 | 61.6 |
| RI_53 | 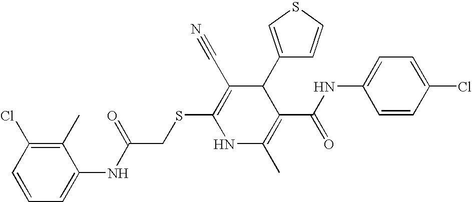 | 56 | 99.2 |
| RI_54 | 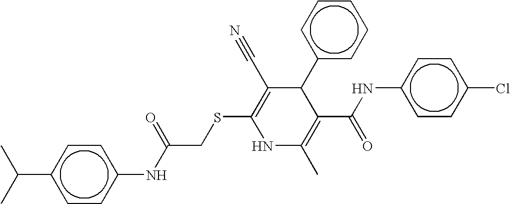 | 45.89 | 52.6 |
| RI_55 | 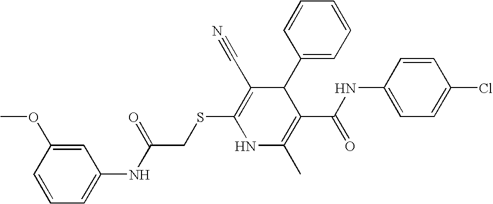 | 48.1 | 40.3 |
| RI_56 | 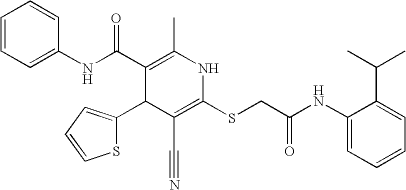 | 54.5 | 100 |
| RI_57 | 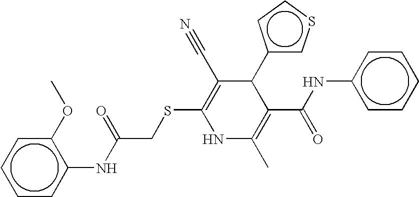 | 49.1 | 37.3 |
| RI_58 | 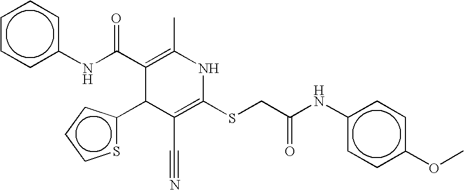 | 23.1 | 25.6 |
| RI_59 | 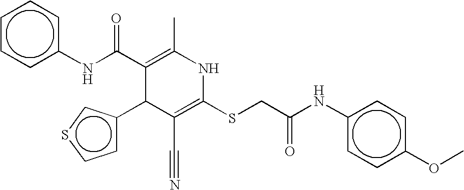 | 27.7 | 30.6 |
| RI_60 | 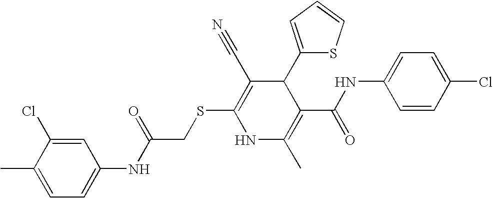 | 59.4 | 71.1 |
| RI_61 | 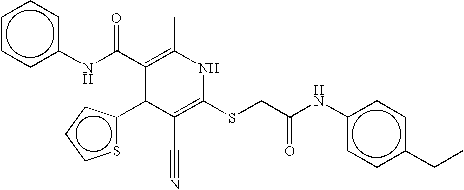 | 68.5 | 51.4 |
| RI_62 | 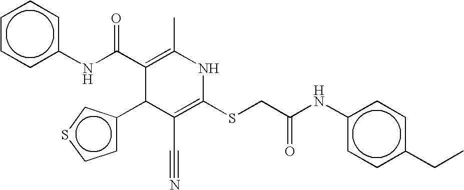 | 91.6 | 62.4 |
| RI_63 | 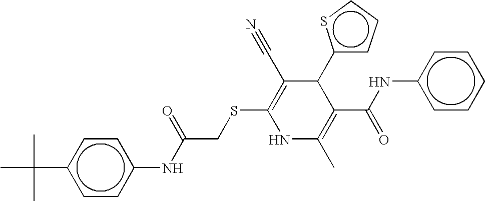 | 38.2 | 38.8 |
| RI_64 | 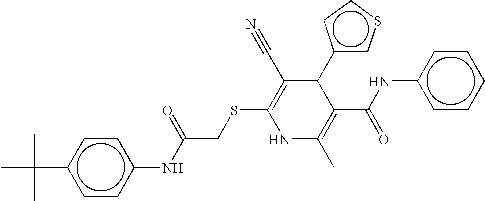 | 34.1 | 41.3 |
| RI_65 | 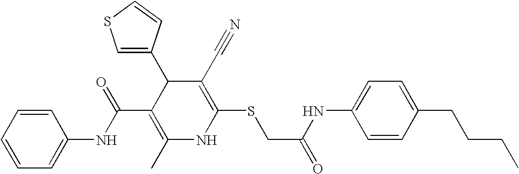 | 51.7 | 38.1 |
| RI_66 | 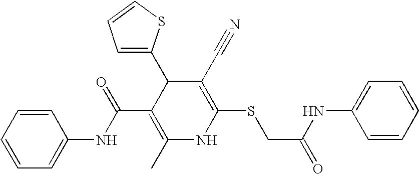 | 20.6 | 23.6 |
| RI_67 | 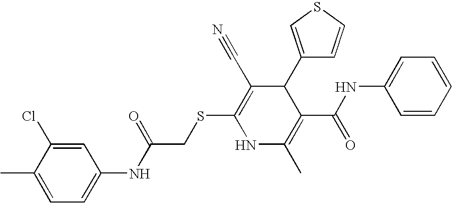 | 42.6 | 51.8 |
| RI_68 | 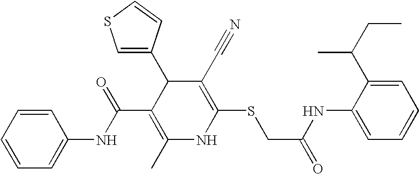 | 74.6 | 100 |
| RI_69 | 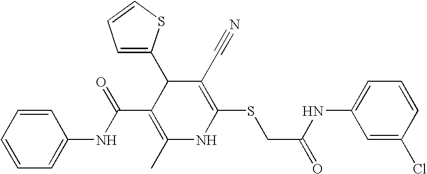 | 89.1 | 69.8 |
| RI_70 | 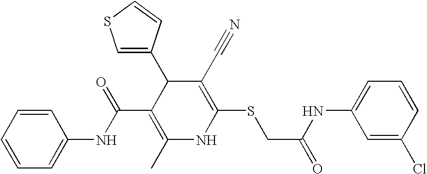 | 74.1 | 58.2 |
| RI_71 | 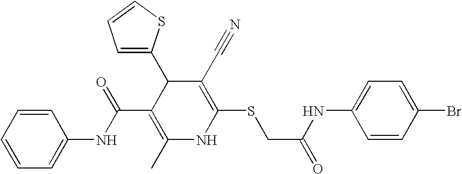 | 34.5 | 25.1 |
| RI_72 | 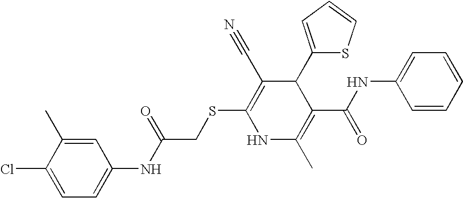 | 40.2 | 50.9 |
| RI_73 | 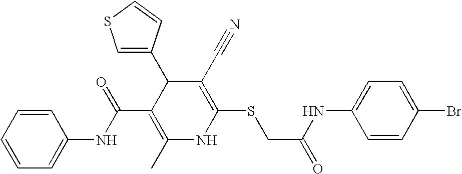 | 24.3 | 34.9 |
| RI_74 | 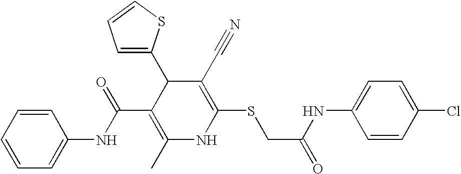 | 31.4 | 41.6 |
| RI_75 | 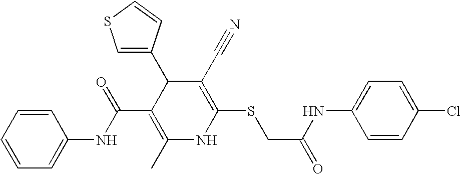 | 27.3 | 38.6 |
| RI_76 | 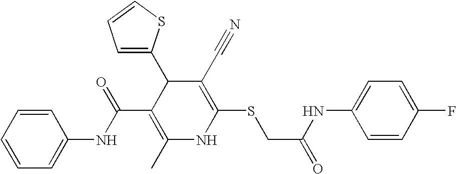 | 46.2 | 54.6 |
| RI_77 | 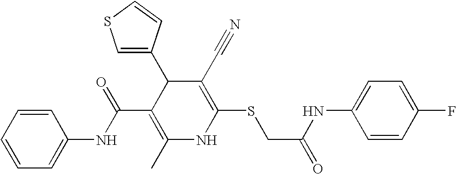 | 16.9 | 25.4 |
| RI_78 | 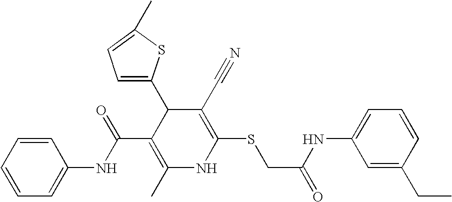 | 77.32 | 88.1 |
| RI_79 | 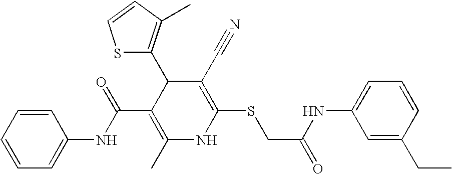 | 55.46 | 51.6 |
| RI_80 | 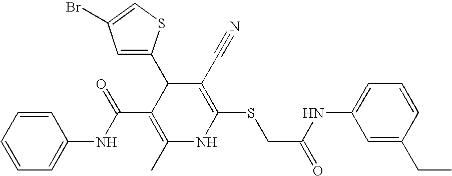 | 64.37 | 54.3 |
| RI_81 | 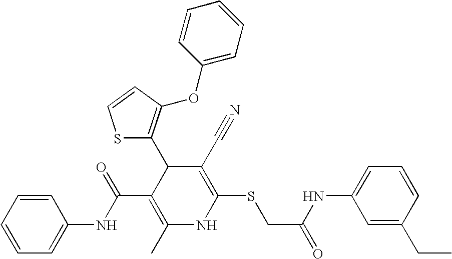 | 73.6 | 100 |
| RI_82 | 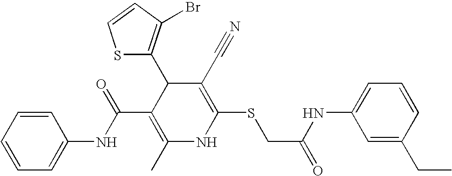 | 67.5 | 67.9 |
| RI_83 | 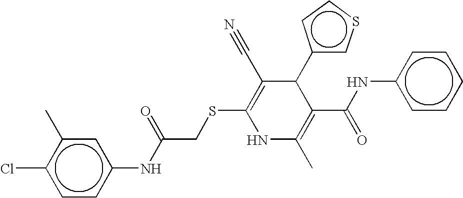 | 44.1 | 55.4 |
| RI_84 | 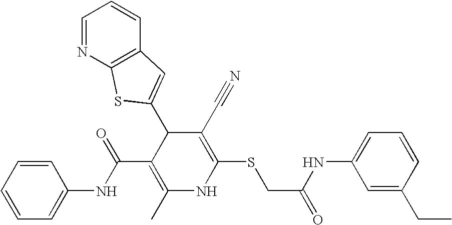 | 64.2 | 44.5 |
| RI_85 | 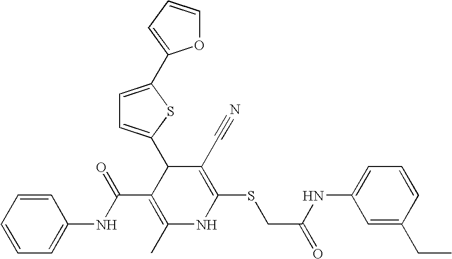 | 98.5 | 46.7 |
| RI_86 | 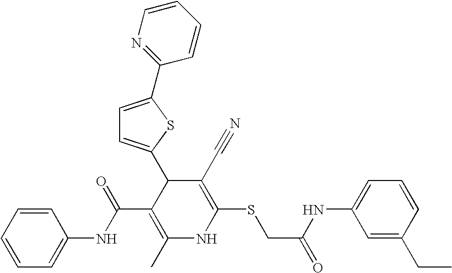 | 32.8 | 21.8 |
| RI_87 | 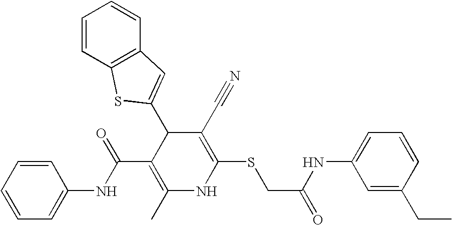 | 77.1 | 49 |
| RI_88 | 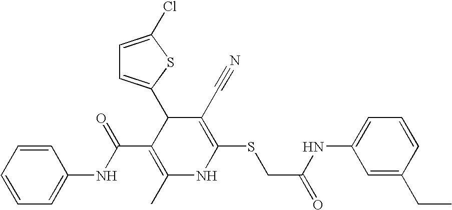 | 70.1 | 54.6 |
| RI_89 | 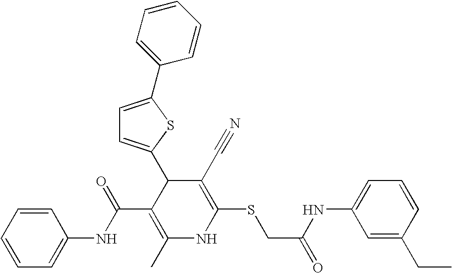 | 63.7 | 55.1 |
| RI_90 | 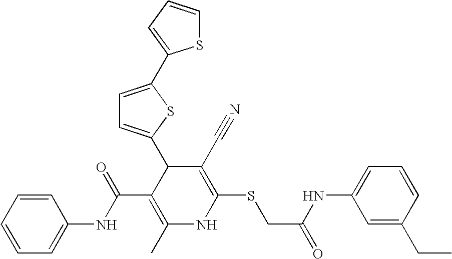 | 63.9 | 42.9 |
| RI_91 | 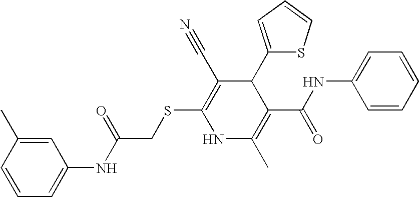 | 55.9 | 60.7 |
| RI_92 | 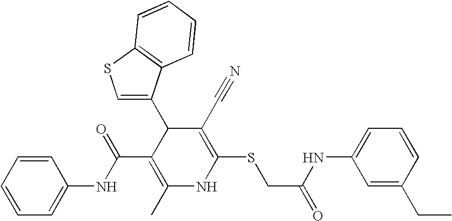 | 60.7 | 47.3 |
| RI_93 | 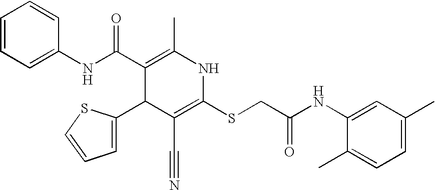 | 42.6 | 58 |
| RI_94 | 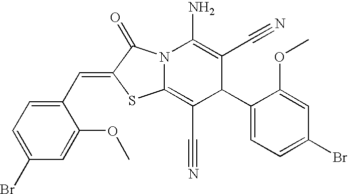 | 73.2 | 81.2 |
| RI_95 | 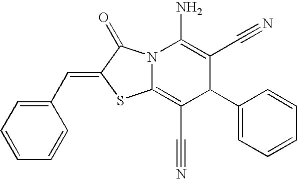 | 67.9 | 69.9 |
| RI_96 | 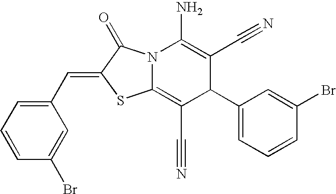 | 71.3 | 70.5 |
| RI_97 | 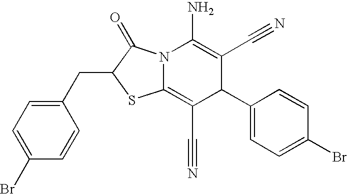 | 76.7 | 74.9 |
| RI_98 | 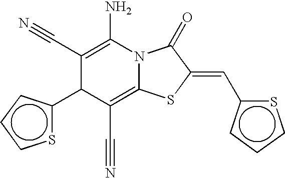 | 74.6 | 78.2 |

TABLE 3
Additional Dihydropyridine Compounds

| | | | Aβ1-40 | Aβ-42 |
|---|---|---|---|---|
| ID | IUPAC Name | Structure | (% of control) | (% of control) |
| ST002431 | 1-[(aminothioxomethyl)amino]-2,6- dimethylhydropyridin-4-one | 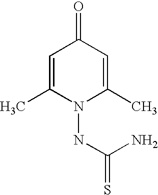 | 16.91194712 | 97.64111454 |
| ST014350 | 4-oxo-2-sulfanyl-6- (trifluoromethyl)hydropyridine-3- carbonitrile, 4-methylmorpholine salt | 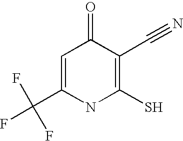 | 81.81151946 | 46.12690315 |
| ST056312 | (2S)-2-amino-3-(3-hydroxy-4- oxohydropyridyl)propanoic acid | 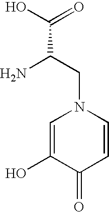 | 70.68288717 | 37.1465806 |
| ST007544 | 3,5-bis(ethoxycarbonyl)-2,6- dimethyl-1,4-dihydropyridine-4- carboxylic acid | 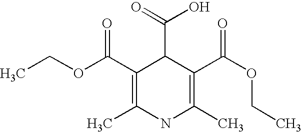 | 78.73644014 | 80.05470709 |
| ST019328 | [2-(1-cyclohexyl-4- hydropyridylidene)ethylidene[methane- 1,1-dicarbonitrile | 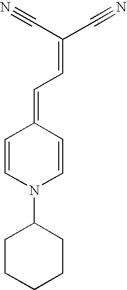 | 59.02172484 | 92.16457961 |
| ST208051 | {2-[1-benzyl-4- hydropyridylidene]ethylidene}methane- 11-dicarbonitrile | 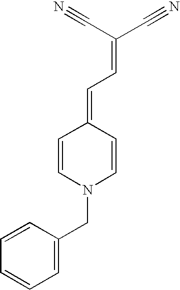 | 57.06516875 | 74.97997576 |
| ST026819 | 2-(5-nitro-2-phenyl-1-(4-pyridyl)(4- 6-hydropyridylidene))-N-(3-pyridyl)- 2-azaace tamide | 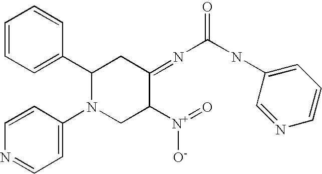 | 64.94486384 | 57.79483456 |
| ST020165 | ethyl 3,6,7,8-tetramethyl-4-pentyl- 6-hydropyrrolo[4,5-f]indolizine-2- carboxyla te, chloride | 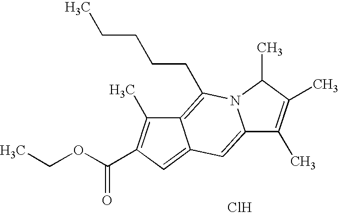 | 90.94364983 | 23.138825 |
| ST044920 | 2-[2-amino-4,4- bis(trifluoromethyl)-3-cyano- 1,4,5,6,7-pentahydrocyclopenta[1,2- b]pyridinyl]-4,5,6,7- tetrahydrobenzo[b]thiophene-3- carbonitrile | 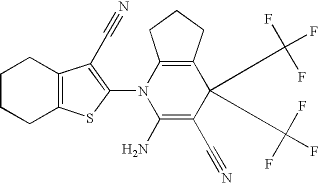 | 76.92072428 | 48.18900702 |

Pharmaceutical Formulations and Methods of Administration

Compounds disclosed herein can be administered in an effective amount for the treatment of a disease associated with cerebral accumulation of β-amyloid, such as Alzheimer's disease, cerebral amyloid angiopathy, hereditary cerebral hemorrhage with amyloidosis Dutch-type, other forms of familial Alzheimer's disease and familial cerebral Alzheimer's amyloid angiopathy. Such compounds are also referred to herein as “active agents.” Dosage amounts and pharmaceutical formulations can be selected using methods known in the art. The compound can be administered by any route known in the art including parenteral, oral or intraperitoneal administration.

The compounds disclosed herein that are administered to animals or humans are dosed in accordance with standard medical practice and general knowledge of those skilled in the art. In particular, therapeutically effective amounts of compounds or more, can be administered in unit dosage form to animals or humans afflicted with a disease associated with cerebral accumulation of Alzheimer's amyloid or suffering from a traumatic brain injury, as well as administered diagnostically for the purpose of determining the risk of developing and/or a diagnosis of a disease associated with cerebral accumulation of Alzheimer's amyloid.

Parenteral administration includes the following routes: intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, or nasal inhalation via insufflation or nebulization. The nasal inhalation is conducted, for example, using aerosols, atomizers or nebulizers.

Examples of suitable dosage amounts are, e.g., about 0.02 mg to 1000 mg per unit dose, about 0.5 mg to 500 mg per unit dose, or about 20 mg to 100 mg per unit dose. The daily dosage can be administered in a single unit dose or divided into two, three or four unit doses per day. The duration of treatment of the active agent is, for example, on the order of hours, weeks, months, years or a lifetime. The treatment may have a duration, for example, of 1-7 days, 1-4 weeks, 1-6 months, 6-12 months, or more.

The compound can be administered to the CNS, parenterally or intraperitoneally. Solutions of compound, e.g., as a free base or a pharmaceutically acceptable salt can be prepared in water mixed with a suitable surfactant, such as hydroxypropylcellulose. Dispersions also can be prepared in glycerol, liquid polyethylene glycols, and mixtures thereof, and in oils. Under ordinary conditions of storage and use, these preparations can contain a preservative and/or antioxidants to prevent the growth of microorganisms or chemical degeneration.

The compounds which are orally administered can be enclosed in hard or soft shell gelatin capsules, or compressed into tablets. The compounds also can be incorporated with an excipient and used in the form of ingestible tablets, buccal tablets, troches, capsules, sachets, lozenges, elixirs, suspensions, syrups, wafers, and the like. Further, compounds can be in the form of a powder or granule, a solution or suspension in an aqueous liquid or non-aqueous liquid, or in an oil-in-water or water-in-oil emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain a compound as disclosed herein, sucrose as a sweetening agent, methyl and propylparabens as preservatives, a dye and flavoring. Additionally, a compound can be incorporated into sustained-release preparations and formulations.

Evaluating Therapeutic Efficacy

Compounds can be evaluated for potential efficacy in the treatment and diagnosis of diseases associated with β-amyloid accumulation using in vitro assays, particularly cultured cell-based assays, and then in vivo assays in animal models using methods known in the art.

Compounds can be tested for a reduction in β-amyloid production in cells exposed to the test compound. In the method, the concentration of β-amyloid (e.g., Aβ1-40 and/or Aβ1-42) in cells exposed to the compound can be measured and compared with a measurement of β-amyloid production in unexposed cells, for example, in a control run in parallel. A decrease in the production β-amyloid, alone or in combination, for example of about 5%, 10%, 15%, 20%, 25%, 30%, 50%, or more in the exposed cells compared to the control cells indicates the potential therapeutic effectiveness of the compound to treat animals or humans afflicted with a disease associated with cerebral accumulation of Alzheimer's amyloid. In one embodiment, total β-amyloid concentration (Aβ1-40+Aβ1-42) is measured. The β-amyloid is measured, e.g. in the culture medium comprising the cells, or intracellularly.

The method of measuring β-amyloid may include testing an array of compounds, e.g., in a 96 well plate, as well as one or more control samples. In the assay, the compound is often required to be incubated with the cells for about 4-48 hours, or e.g., 18-36 hours. β-amyloid can be detected using an ELISA sandwich assay using quantitatively commercially available enzymatically labeled (with horseradish peroxidase) antibodies to Aβ1-40 and Aβ1-42 as described in the Example. The labeled antibody ELISA assay also can require on the order of 24 hours to complete. The compounds which are tested for their ability to reduce AB production may be screened in a range of concentrations, for example of about 1 nM to 10 mM, about 500 nM to 50 μM, or about 5 μM to 30 μM.

Cells which can be used in the assays described herein for measuring a reduction in β-amyloid production include mammalian or non-mammalian cells that overexpress APP or a fragment thereof, including but not limited to Chinese hamster ovary (CHO) cells, for example, 7W WT APP751 CHO cells. See, e.g., Koo and Squazzo, J. Biol. Chem., Vol. 269, Issue 26, 17386-17389, July, 1994. Cell lines transfected with APP have been described in the art and include 7W (wt APP₇₅₁); 7W_{ΔC} (APP₇₅₁ with deletion of almost the entire cytoplasmic tail (residue 710-751); 7W_{SW} (APP₇₅₁ with the “Swedish” KM651/652NL double-mutation); and 7W_{VF} (APP₇₅₁ with the V698F mutation). See, e.g. Xia et al., Proc. Natl. Acad. Sci. USA 94:8208-8213, 1997; and Pérez, R. & Koo, E. (1997) in Processing of the β-Amyloid Precursor Protein Effects of C-Terminal Mutations on Amyloid Production, eds. Iqbal, K., Winblad, B., Nishimura, T., Takeda, M. & Wisniewski, H. M. (J. Wiley & Sons, London), pp. 407-416. The APP which is overexpressed can include transcripts of APP, such as, without limitation, APP751.

Compounds can also be tested using transgenic animal models for example, for AD, such as, without limitation, PDAPP and TgAPPsw mouse models, which can be useful for screening compounds for ability to reduce β-amyloid production, β-amyloid deposition, β-amyloid neurotoxicity (including abnormal hyperphosphorylation of tau) and microgliosis in the central nervous system of such animals or in humans. Transgenic animal models for AD can be constructed using standard methods known in the art, as set forth for example, without limitation, in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; and 4,736,866.

Also provided is the use of a compound selected from Tables 1, 2, and 3, for the manufacture of a medicament for the treatment of a disease associated with cerebral accumulation of Alzheimer's amyloid, such as Alzheimer's disease (AD).

### EXAMPLES

The invention will be understood in further detail in view of the following non-limiting examples.

### Example 1

### Measurement of Aβ1-40 and Aβ1-42

1. Materials and Methods

Chinese hamster ovary (CHO) cells, stably transfected with human APP751 (7W WT APP751 CHO cells) were used. See, e.g., Koo and Squazzo, J. Biol. Chem., 269(26): 17386-17389, 1994. The cells were maintained in DMEM medium supplemented with 10% fetal bovine serum and 1× mixture of penicillin/streptomycin/fungizone/glutamine mixture (Cambrex, Md.) geneticin as selecting agent in 75 cm² cell culture flasks.

The 7W WT APP751 CHO cells were plated in 96-well cell culture plates in quadruplicate, containing 200 microliters of culture medium, for 18 hours at 37° C. and 5% CO₂. All test compounds were placed in dimethyl sulfoxide (DMSO) before being added to the cultured confluent 7W WT APP751 CHO cells to a concentration of the test compound of 5 μM. The culture medium was collected and diluted before being assayed by ELISAs for Aβ1-40 at 10-fold dilution and Aβ1-42 at 2-fold dilution, respectively. Concentrations of Aβ1-40 and Aβ1-42, expressed in pg/ml, were determined using commercially available ELISAs (Biosource, Calif.) in a colorimetric assay using labeled antibodies detected spectrophotometrically. For compounds with an identification beginning “RI”, the compounds were tested at a concentration of 10 μM for 24 hours. Control cells were treated with DMSO containing no compound.

2. Results

The percentage reduction in levels of Aβ1-40 and Aβ1-42 as compared to control cells not exposed to the test compounds are reported for the compounds in Tables 1, 2, and 3, supra. An entry of “0” indicates no detectable amount of Aβ1-40 or Aβ1-42 according to the assay conditions. The data in Tables 1, 2, and 3 may be rounded to the nearest 0.1%.

### Example 2

### Synthesis of Compounds

General techniques: All reactions requiring anhydrous conditions were conducted in oven-dried glass apparatus under an atmosphere of nitrogen. Preparative chromatographic separations were performed on Combiflash Companion, Isco Inc.; reactions were followed by TLC analysis using silica plates with fluorescent indicator (254 nm) and visualized with UV, phosphomolybdic acid or 4-hydroxy-3-methoxybenzaldehyde. All commercially available reagents were purchased from Aldrich and Acros and were typically used as supplied.

Purity of compounds was checked with Agilent 1100 series system using analytical HPLC column (Eclipse XDB-C18, 5 micron, 4.6 mm i.d) in two different solvent systems (methanol/water and acetonitrile/water) using a gradient program and found to be >98% pure. ¹H and ¹³C NMR spectra were recorded in Fourier transform mode at the field strength specified on a Varian AS500 spectrometer and chemical shifts are expressed in ppm relative to tetramethylsilane as an internal standard. Multiplicities in the ¹H NMR spectra are described as: s=singlet, d=doublet, t=triplet, q=quartet, m=multiplet, br=broad; coupling constants are reported in Hz. Low (MS) resolution mass spectra were measured on a Micromass Q-T of API-US spectrometer utilizing an Advion Bioscience Nanomate electrospray source. Ion mass/charge (m/z) ratios are reported as values in atomic mass units.

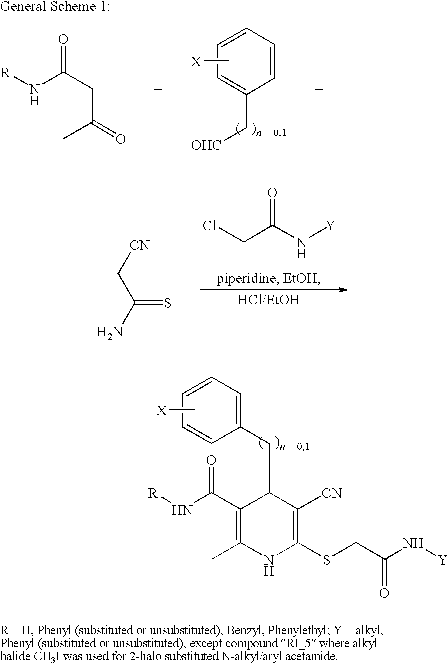

General Synthesis:

Piperidine (1.1 mol equivs.) was added to a solution of 3-oxo-N-pheylbutanamide

(1.0 mol equiv.), substituted/unsubstituted alkyl/aryl aldehyde (1.0 mol equiv.) and 2-cyanothioacetamide (1.0 mol equiv.) in absolute EtOH (12.5 or 25 mL). The reaction mixture was sequentially heated (<5 min), cooled to room temperature and the 2-halo substituted N-alkyl/aryl acetamide (1.1 mol equiv.) (except where 2-halo substituted N-alkyl/aryl acetamide═CH₃I) was added. The resulting solution was heated (<10 min) and cooled to room temperature. The product was precipitated by adding HCl/EtOH (3 M) to the reaction mixture and refluxing for 5 min. The reaction mixture was then cooled to room temperature. The precipitate so formed was filtered, rinsed with H₂O, EtOH, hexane/EtOAc (1:1) or hexane, and dried under vacuum with heat to afford the desired product.

Synthesis of Cyclic Analog of STO51153:

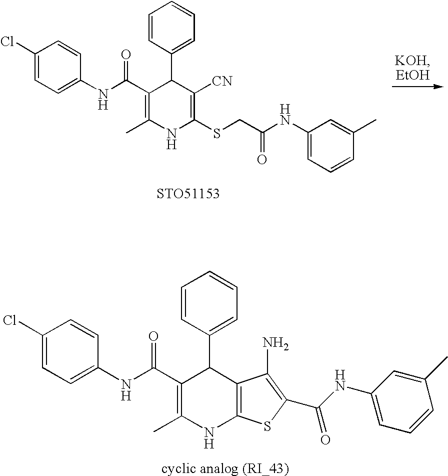

To suspension of compound ST51153 (1 mmol) in EtOH (10 mL), aqueous KOH (4 M in H₂O, 0.5 mL) was added dropwise. The reaction mixture was heated to reflux for 10 min and then stirred at room temperature for 45 min. A precipitate formed which was filtered, rinsed with H₂O (3×10 mL) and hexane (5×10 mL), and dried under vacuum and heat to afford compound “RI_{—}43” as a yellow solid (0.320 g, 61% yield).): ¹HNMR (DMSO-d₆) δ 2.32 (s, 3H, CH₃), 2.68 (s, 3H, CH₃), 5.77 (s, 2H, NH₂), 6.91-7.50 (m, 13H, ArH), 9.49 (s, 1H, NH), 10.52 (s, 1H, NH). Ref: Heterocylic Communications 2001, 7(4), 375-380.

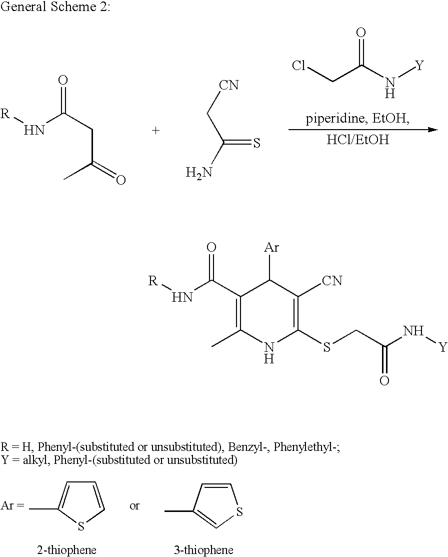

General Synthesis:

Piperidine (1.1 mol equivs) was added to a solution of 3-oxo-N-pheylbutanamide (1.0 mol equiv.), 2 or 3-thiophenecarboxaldehyde (1.0 mol equiv) and 2-cyanothioacetamide (1.0 mol equiv.) in absolute EtOH (12.5 or 25 mL). The reaction mixture was sequentially heated (<5 min), cooled to room temperature and the 2-halo substituted N-alkyl/aryl acetamide (1.1 mol equiv) was added. The resulting solution was heated (<10 min) and cooled to room temperature. The product was precipitated by adding HCl/EtOH (3 M) to the reaction mixture and refluxation for 5 min. The reaction mixture was then cooled to room temperature. The precipitate so formed was filtered, rinsed with H₂O, EtOH, hexane/EtOAc (9:1) or hexane, and dried under vacuum with heat to afford the desired product.

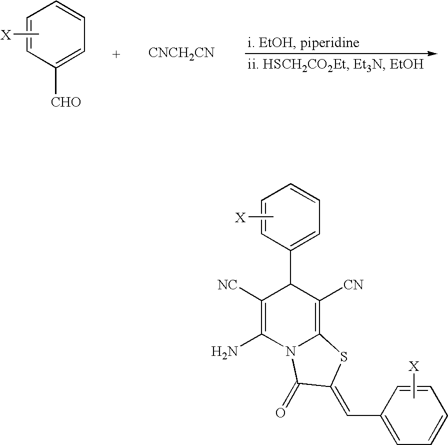

General Synthesis:

The substituted/unsubstituted aryl aldehyde (1.0 mol equiv.) was added to malononitrile (1.0 mol equiv.) in absolute EtOH followed by a few drops of piperidine. The reaction mixture was then heated at reflux for 2 h, half the solvent was removed, a drop of hexane was added and a precipitate formed. The precipitate was filtered and rinsed with hexane to afford the crude benzylidenemalononitrile intermediate. Triethylamine (1.0 mol equiv.) was then added to the above intermediate (2.0 mol equivs.) in EtOH and the resulting mixture heated at reflux for 2 h. A precipitate formed, was filtered, rinsed with H₂O, EtOH and hexane to afford the desired product.

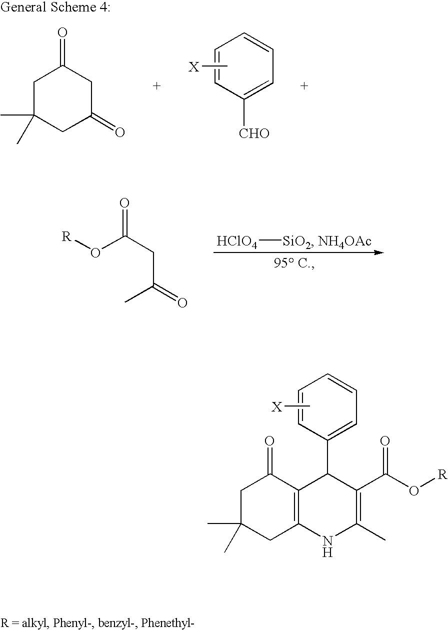

General Synthesis:

5,5-dimethyl-1,3-cyclohexanedione (1.0 mol equiv.), aldehyde (1.0 mol equiv.), benzylacetoacetate (1.0 mol equiv.), 0.5 mmol/g HClO₄—SiO₂ (0.250 g), and NH₄OAc (1.5 mol equivs.) mixture was heated at 95° C. for 1 h. Ethyl acetate was added, the mixture was heated to dissolve solids, cooled, filtered and concentrated. Hexane/EtOAc (9:1) was added to the crude product and vigorously stirred at room temperature until all solvent evaporated leaving behind a yellow solid. The solid was suspended in the same solvent, filtered, collected and purified by flash chromatography using hexane/EtOAc (1:1) to afford the product.

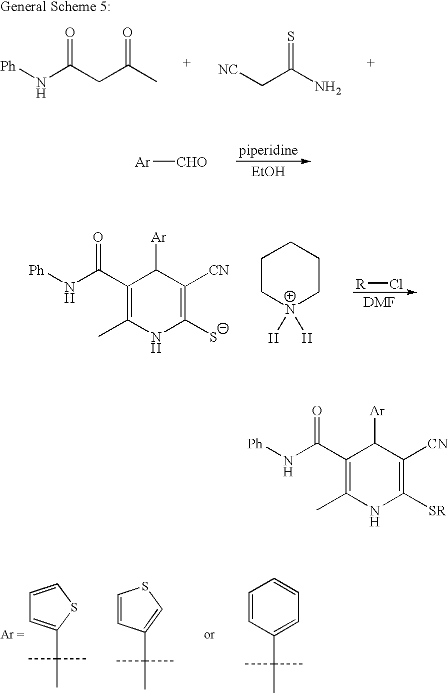

General Synthesis:

Acetoacetaniline (3.58 g, 20 mmol) and 2-cyanothioacetamide (2.06 g, 20 mmol) were dissolved in absolute EtOH (20 mL). To this solution was added the corresponding carboxaldehyde (20 mmol) dropwise, followed by piperidine (2.0 mL). The resulting suspension was heated to reflux for 1 h and cooled down to rt. The yellow solid was filtered and washed with EtOH and hexanes and dried, affording the corresponding salt. 200 mg of this salt and 2-chloroacetamide (1 equiv.) were dissolved in dry DMF (2 mL). The solution was heated to 90° C. for 2 h and poured into ice. The resulting solid was filtered and recrystallization from EtOH gave the titled compound.

It should be understood that the embodiments described herein are for illustrative purposes only and that various modifications or changes in light thereof will be suggested to persons skilled in the art and are to be included within the spirit and purview of this application. All references cited herein are incorporated by reference in their entirety.

What is claimed is:

1. A method for treating, slowing the progression of, or delaying the onset of a disease associated with cerebral accumulation of Alzheimer's amyloid in an animal or human subject in need thereof, said method comprising administering to said animal or human subject a therapeutically effective amount of a compound according to Formulas I or II or selected from the compounds listed in Tables 1, 2, and 3, and pharmaceutically acceptable derivatives, salts and prodrugs thereof.

2. The method of claim 1, wherein the subject is a human

3. The method of claim 1 or 2, wherein the disease is Alzheimer's Disease.

4. The method of claim 1 wherein the disease is not Alzheimer's Disease.

5. The method of claim 4, wherein the disease is cerebral amyloid angiopathy, hereditary cerebral hemorrhage with amyloidosis Dutch-type, other forms of familial Alzheimer's disease and other forms of familial cerebral Alzheimer's amyloid angiopathy.

6. The method of claim 2, wherein the subject is elderly.

7. The method of claim 1, wherein the subject has a predisposition to developing a disease associated with cerebral accumulation of Alzheimer's amyloid.

8. The method of claim 2, wherein the subject has a family history of early onset Alzheimer's Disease or an ApoE epsilon 4 genotype, or both.

9. The method of claim 3, wherein the subject has early stage Alzheimer's Disease.

10. A method for the treatment of an animal or human subject suffering from traumatic brain injury, said method comprising administering a therapeutically effective amount of a compound according to Formulas I or II or selected from the compounds listed in Tables 1, 2, and 3, and pharmaceutically acceptable derivatives, salts and prodrugs thereof.

11. The method of claim 10, wherein said compound is administered within 1 hour, within 2 hours, or within 8 hours after said traumatic brain injury.

12. The method of claim 11, wherein said compound is further continued to be administered for a period thereafter.

13. The method of claim 1 or 10, wherein the therapeutically effective amount of the compound is selected from an amount between about 0.02 to 1000 mg per unit dose, and between about 0.5 to 500 mg per unit dose.

14. The method of claim 1 or 10, wherein the duration of treatment with the compound lasts for up to the lifetime of the animal or human.

15. The method of claim 1 or 10, wherein the route of administration of the compound to the animal or human is parenteral, oral or intraperitoneal.

16. The method of claim 1 or 10, wherein the compound is administered orally in a unit dosage form selected from the group consisting of hard or soft shell gelatin capsules, tablets, troches, sachets, lozenges, elixirs, suspensions, syrups, wafers, powders, granules, solutions and emulsions.

17. The method of claim 1 or 10, wherein the compound is administered parenterally by a route of administration selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; and topical, including ophthalmic, dermal, ocular, rectal, and nasal inhalation via insufflation or nebulization.

18. A pharmaceutical composition comprising a compound according to Formulas I or II or selected from the compounds listed in Tables 1, 2, and 3, and pharmaceutically acceptable derivatives, salts and prodrugs thereof in an amount effective to treat a disease or disorder associated with cerebral accumulation of Alzheimer's amyloid or traumatic brain injury; and a pharmaceutically acceptable carrier.

19. The pharmaceutical composition of claim 18, which is formulated for parenteral, oral or intraperitoneal administration.

20. The pharmaceutical composition of claim 18, which is in a dosage form selected from the group consisting of hard or soft shell gelatin capsules, tablets, troches, sachets, lozenges, elixirs, suspensions, syrups, wafers, powders, granules, solutions and emulsions.

21. The pharmaceutical composition of claim 18, which is formulated for parenteral administration by a route selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; and topical, including ophthalmic, dermal, ocular, rectal, and nasal inhalation via insufflation or nebulization.

22. A unit dosage form comprising a pharmaceutical composition comprising a compound according to Formulas I or II or selected from the compounds listed in Tables 1, 2, and 3, and pharmaceutically acceptable derivatives, salts and prodrugs thereof in an amount effective to treat a disease or disorder associated with cerebral accumulation of Alzheimer's amyloid or traumatic brain injury; and a pharmaceutically acceptable carrier.

23. The unit dosage form of claim 22, wherein the pharmaceutical composition is formulated for parenteral, oral or intraperitoneal administration.

24. The unit dosage form of claim 22, which is in a form selected from the group consisting of hard or soft shell gelatin capsules, tablets, troches, sachets, lozenges, elixirs, suspensions, syrups, wafers, powders, granules, solutions and emulsions.

25. The unit dosage form of claim 22, wherein the pharmaceutical composition is formulated for parenteral administration by a route selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; and topical, including ophthalmic, dermal, ocular, rectal, and nasal inhalation via insufflation or nebulization.

26. The unit dosage form of claim 22, wherein the amount of the compound is selected from between about 0.02 to 1000 mg, and between about 0.5 to 500 mg.

27. A method for reducing Aβ deposition, Aβ neurotoxicity and microgliosis in an animal or human afflicted with a cerebral amyloidogenic disease or condition or a traumatic brain injury, comprising administering to the animal or human in need thereof a therapeutically effective amount a compound according to Formulas I or II or selected from the compounds listed in Tables 1, 2, and 3, and pharmaceutically acceptable derivatives, salts and prodrugs thereof.
